(19) United States
(12) Patent Application Publication
(10) Pub. No.: US 2025/0207114 A1
(43) Pub. Date: Jun. 26, 2025

(54) DEVELOPMENT OF RNA-TARGETED GENE EDITING TOOL

(71) Applicant: SHANGHAI GENEMAGIC BIOSCIENCES CO., LTD., Shanghai (CN)

(72) Inventors: Haibo ZHOU, Shanghai (CN); Zhengzheng XU, Shanghai (CN); Shicheng CAI, Shanghai (CN)

(73) Assignee: SHANGHAI GENEMAGIC BIOSCIENCES CO., LTD., Shanghai (CN)

(21) Appl. No.: 18/847,042

(22) PCT Filed: Mar. 14, 2023
(86) PCT No.: PCT/CN2023/081440

§ 371 Date: Sep. 13, 2024

(30) Foreign Application Priority Data
Mar. 14, 2022 (CN) 202210246868.4

(51) Int. Cl. C12N 9/22; C12N 15/11; C12N 15/86

(52) U.S. Cl. CPC C12N 9/22; C12N 15/111; C12N 15/86; C07K 2319/00; C12N 2310/20

(57) ABSTRACT

A method for screening a compact Cas13 protein and the use. The present disclosure relates to the fields of biotechnology and medicine. More specifically, the content of the present disclosure relates to a new Cas13 family protein, a method for screening the new Cas13 family protein, and a corresponding RNA editing system and the use thereof. The content of the present disclosure particularly relates to a Cas13 protein and a related RNA editing system. The molecular weight of the new Cas13 protein is very low, which almost pushes a CRISPR-Cas protein having guide RNA guidance and RNase activity to the limit, and contains more extended HEPN domains. According to the content of the present disclosure, a screening method for rapidly searching for CRISPR-Cas13 proteins that have an ultra-low molecular weight, are dependent on guide RNA guidance and have RNase activity is provided for the first time, and a variety of new Cas13 proteins and new families thereof are obtained, which have broad application prospects and huge market values.

This application claims priority to application number CN202210246868.4, titled “DEVELOPMENT OF RNA-TARGETED GENE EDITING TOOL”, filed on Mar. 14, 2022, the entire content of which is incorporated by reference herein in its entirety.

### TECHNICAL FIELD

This present disclosure relates to the fields of biotechnology and medicine. More specifically, the present disclosure relates to new Cas13 protein family, method of screening new Cas13 protein family, as well as corresponding RNA editing systems and their applications. The present disclosure particularly relates to low-molecular-weight Cas13 proteins and the corresponding RNA editing systems.

### BACKGROUND TECHNIQUE

The CRISPR-Cas system, known as a key component of the new generation of genome engineering tools, plays the role of an adaptive immune mechanism in microorganisms such as bacteria and archaea, safeguarding microorganisms against viruses and other foreign nucleic acids. The CRISPR-Cas immune response mainly includes three stages: adaptation stage, expression and processing stage, and interference stage. Similar to other defense mechanisms, CRISPR-Cas systems evolve in the context of constant competition with mobile genetic elements, which leads to extreme diversity in Cas protein sequences and CRISPR-Cas locus structures.

Since 2011, CRISPR-Cas systems have been classified into two categories based on methods such as genetic constitution, locus structure, and sequence similarity clustering of the CRISPR-Cas system. The first category is the effector module composed of multiple Cas proteins, some of which form crRNA-binding complexes that mediate pre-crRNA processing and interference through additional Cas proteins. The second category contains a single Cas effector protein with a multifunctional domain binding region that can bind to crRNA and participate in all activities necessary for interference. Some variants also participate in the maturation process of pre-crRNA. The second category is mainly divided into 3 subtypes: type II (such as Cas9), type V (such as Cas12a), type VI (such as Cas13d). The subtype of type II and type V mainly target DNA, and type VI effector Cas proteins mainly target RNA.

Currently, various CRISPR-Cas-dependent gene editing tools have been developed based on the CRISPR-Cas system of the second category, including CRISPRa, CRISPRi, and base editing technology, etc. However, the delivery of genes into cells requires delivery tools. Commonly used delivery tools include retroviruses, adenoviruses or adeno-associated viruses, etc. These tools have limited carrying capacity, for example, the adeno-associated virus (AAV) can't accommodate DNA exceeding 4.7 kb, so that it is disadvantageous for the packaging of large molecular weight CRISPR-Cas related tools.

In 2020, researchers found a Cas @ protein (also classified as Cas12j subfamily) with a molecular weight only half of Cas9 and Cas12a genome editing enzymes in huge bacterial virus phages. It is capable of cleaving DNA in eukaryotic cells. Recently, Zhang Feng's team also found the ancestor protein IscB (about 400 amino acids) and TnpB family of Cas9 and Cas12. But these are DNA-targeted enzymes. Currently, the known smallest Cas13 effector proteins capable of editing RNA, such as Cas13bt, Cas13X, etc., all exceed 700 amino acids.

Previous research strategies mainly based on the sequence conservation of Cas1 protein to determine the neighboring Cas protein. However, this approach may miss some single-effector proteins which have no Cas1 protein. Based on the coexistence of CRISPR-array and Cas protein, scholars are prompted to start directly by predicting CRISPR array, and then search for neighboring CRISPR-Cas related protein. Nevertheless, due to the limitation of the current algorithm for predicting CRISPR array, no algorithm has been universally recognized as the gold standard. In addition, the identification of candidate proteins mainly relies on DNA and protein sequence comparison, which can easily ignore the impact of protein spatial folding. Therefore, there is an urgent need to develop new methods for screening single effector proteins related to the CRISPR-Cas13 system with smaller molecular weight and new cas13 proteins with smaller molecular weight.

### Contents

In view of the shortcomings and actual needs of existing technologies for screening new CRISPR-Cas proteins, this disclosure provides a method to quickly search for new guide RNA-guided CRISPR-Cas13 proteins with RNase activity that contain multiple (at least one) extended HEPN domains. The RNase activity of the candidate proteins is verified both from the perspective of bioinformatic analysis (such as sequence alignment, protein structure prediction, etc.) and experimental validation. These proteins are potentially used in RNA-level regulation, editing, detection, etc., and have broad academic value and commercial application value.

The technical problem solved by this disclosure is how to quickly find candidate CRISPR-Cas13 proteins and their systems with more novel RNA enzyme cleavage activity domains (extended HEPN domains). Then, the problem solved is verification the activity of these candidate CRISPR-Cas13 proteins and their systems. Ultimately, a variety of novel Cas13 proteins have been obtained.

In a first aspect of the present disclosure, Cas13 proteins are provided. the Cas13 proteins comprise amino acid sequence shown as any one of SEQ ID NO: 1 to 78, or comprise the protein having at least 70%, 80%, 85%, 90%, or 95% homology with the sequence of any one of SEQ ID NO: 1 to 78. Preferably, the proteins comprise amino acid sequence shown as any one of SEQ ID NOs: 1-34, 37, 38, 41, 42, 43, 45, 46, 47, 49, 52, 54, 55, 58, 61, 62, 64, 65, or 68-71, or comprise the protein having at least 70%, 80%, 85%, 90%, or 95% homology with the sequence shown as any one of SEQ ID NO: 1-34, 37, 38, 41, 42, 43, 45, 46, 47, 49, 52, 54, 55, 58, 61, 62, 64, 65, or 68-71. More preferably, the proteins comprise amino acid sequence shown as any one of SEQ ID NO: 1, 3, 6, 17, 19, 21, 27, 31, 33, 55, 68, 69, and 71, or comprise the protein having at least 80%, 85%, 90%, or 95% homology with the sequence shown as any one of SEQ ID NO: 1, 3, 6, 17, 19, 21, 27, 31, 33, 55, 68, 69, 71.

In a preferred embodiment, the Cas13 proteins according to the first aspect of the present invention, wherein the protein having at least 80%, 85%, 90%, or 95% homology refers to the protein having conservative amino acid addition, deletion, or substitution of one or more residues; preferably, refers to the protein having conservative amino acid addition, deletion, or substitution of 1-10 residues.

In a second aspect of the present disclosure, the Cas13 proteins are provided, wherein the HEPN domain of the proteins comprise at least one RXXXXXH and/or RXXXXXXH motif, wherein X represents an optional amino acid. Preferably, HEPN domain comprises from one to nine RXXXXXH and/or RXXXXXXH motifs. More preferably, HEPN domain comprises from two, three, four, or five RXXXXXH and/or RXXXXXXH motifs.

In a preferred embodiment, in the cas13 proteins provided in the second aspect, the amino acid X adjacent to R is preferably N, Q, H or D.

In a preferred embodiment, the HEPN structure of the cas13 proteins described in the second aspect contains the HEPN structure shown in Table 2.

In a preferred embodiment, the RNA cleavage activity of the cas13 proteins described in the first or second aspect of the present invention is retained.

In a preferred embodiment, the Cas13 proteins according to any one of the first aspect or the second aspect of the present invention, the HEPN domain of the Cas13 proteins has at least one nucleotide mutation.

In a preferred embodiment, the Cas13 protein according to any one of the first aspect or the second aspect of the present invention is fused with one or more heterologous functional domains, wherein the fusion is performed at N-terminal, C-terminal or internal of the Cas13 protein; preferably, the heterologous functional domain has the following activities: deaminase such as cytidine deaminase and deoxyadenosine deaminase, methylase, demethylase, transcriptional activation, transcriptional repression, nuclease, single-stranded RNA cleavage, double-stranded RNA cleavage, single-stranded DNA cleavage, double-stranded DNA cleavage, DNA or RNA ligase, reporter protein, detection protein, localization signal, or any combination thereof.

In a preferred embodiment, the HEPN domain of the cas13 protein according to any one of the first or second aspects of the present invention is identical to the HEPN domain of any one of the sequences shown in SEQ ID NO: 1 to 78.

In a preferred embodiment, at least one of the HEPN domains of the cas13 protein according to any one of the first aspect or the second aspect of the present invention contains RXXXXH, RXXXXXH, and/or RXXXXXXH motifs, wherein X is an optional amino acid. Preferably, the amino acid adjacent to R is N, Q, H or D.

In a preferred embodiment, the aforementioned HEPN domain of cas13 protein contains at least one RXXXXXH and/or RXXXXXXH motif; preferably, the HEPN domain contains 1-9 RXXXXXH and/or RXXXXXXH motifs; more preferably, the cas13 protein contains 2, 3, 4, or 5 HEPN domains.

In a third aspect of the present invention, nucleic acid molecule is provided, wherein the nucleic acid molecule comprises a nucleotide sequence encoding the Cas13 protein according to any one of the first and second aspects of the present invention.

In a preferred embodiment, the nucleic acid molecule is a codon-optimized nucleic acid for a specific host cell; preferably, the host cell is prokaryotic cell or eukaryotic cell; more preferably is eukaryotic cell, and even more preferably is human source cell.

In a preferred embodiment, any of the aforementioned nucleic acid molecules includes a promoter effectively linked to the nucleotide sequence encoding Cas13, and the promoter is constitutive promoter, inducible promoter, tissue-specific promoter, chimeric promoter, or developmental specific promoter.

In the fourth aspect of the present invention, CRISPR-Cas system is provided, the system comprises: (1) the Cas13 protein or derivative or functional fragment thereof according to any one of the first or second aspects of the present invention, or the nucleic acid molecule according to any one of the third aspect of the present invention; and (2) a gRNA targeting to target nucleic acid.

Preferably, the gRNA sequence includes a direct repeat (DR) sequence and a spacer sequence that is complementary to the target nucleic acid.

More preferably, the DR sequence includes the nucleic acid shown in any one of SEQ ID NO: 79-234, or includes the derived nucleic acid from any one of SEQ ID NO: 79-234;
the sequence of the derived nucleic acid is:
(i) a sequence that has one or more (e.g., 1, 2, 3, 4, 5, 6, 7, 8, 9, or 10) nucleotide addition, deletion, or substitution compared to any of the sequences shown in Table 1;
(ii) a sequence that has at least 20%, 30%, 40%, 50%, 60%, 70%, 80%, 90%, 95% or 97% sequence identity to any one of the sequences shown in Table 1;
(iii) a sequence that hybridize with any of the sequences shown in Table 1, or with any one of those in (i) and (ii) under stringent conditions; or
(iv) the complement of any one of sequence shown (i)-(iii), the condition is the said derived nucleic acid is not any of the sequences shown in Table 1, and encodes an RNA or is an RNA, said RNA substantially maintains the same secondary structure as any RNA encoded by any one of SEQ ID NO: 79-234.

In a preferred embodiment, in any of the aforementioned CRISPR-Cas systems, the spacer sequence has 15-60 nucleotides, preferably has 25-50 nucleotides, more preferably has 30 nucleotides.

In a preferred embodiment, the target nucleic acid acted upon by any of the aforementioned CRISPR-Cas systems is target RNA; preferably, the target RNA is mRNA or ncRNA, including non-coding RNA selected from the group consisting of lncRNA, miRNA, misc_RNA, Mt_rRNA, Mt_tRNA, rRNA, scaRNA, scRNA, snoRNA, snRNA, or sRNA.

In the fifth aspect of the present invention, a carrier is provided, the carrier comprises the nucleic acid molecule described in any one of the third aspects and is capable of expressing the Cas13 protein described in any one of the first or second aspects of the present invention or capable of expressing the nucleic acid molecule of any one of the third aspects of the invention; preferably, the carrier is selected from viral vector, lipid nanoparticle (LNP), liposome, cationic polymer (such as PEI), nanoparticle, exosome liposome, microvesicle, gene gun; more preferably, the carrier is selected from viral vector, more preferably, the viral vector is selected from adeno-associated virus (AAV), adenovirus, lentivirus, retrovirus, herpes simplex virus, and oncolytic virus.

In a sixth aspect of the present invention, a delivery system is provided, comprises (1) the carrier described in any one of the fifth aspects, or the nucleic acid molecule described in any one of the third aspects, and (2) a delivery carrier.

In a preferred embodiment, the delivery carrier of the delivery system described in this aspect is nanoparticle, liposome, exosome, microvesicle or gene gun.

In the seventh aspect of the present invention, cell is provided, the cell comprises the Cas13 proteins described in any one of the first or second aspects of the present invention, the nucleic acid molecule described in any one of the third aspect of the present invention, the carrier described in the fifth aspect of the present invention, the delivery system described in the sixth aspect of the present invention, or the CRISPR-Cas system described in any one of the fourth aspect of the present invention.

In a preferred embodiment, the cell described in any one of the aspects is prokaryotic cell or eukaryotic cell, preferably is human cell.

In the seventh aspect of the present invention, methods are provided for degrading or cutting target RNA in target cells or modifying the sequence of target RNA in target cells, which include using the Cas13 proteins described in any one of the first or second aspects of the present invention, the nucleic acid molecule described in any one of the third aspect of the present invention, the carrier described in the fifth aspect of the present invention, the delivery system according to the sixth aspect of the present invention, or the CRISPR-Cas system described in the fourth aspect of the present invention.

In a preferred embodiment, the target cells described in any one of this aspect are prokaryotic cells or eukaryotic cells, preferably are human cells.

In a preferred embodiment, the target cells described in any one of this aspect are ex vivo cells, in vitro cells or in vivo cells.

In the seventh aspect of the present invention, a method for screening Cas13 proteins is provided, which involves selecting Cas13 proteins which contain at least one RXXXXXXH and/or RXXXXXXH motif within their HEPN motif, wherein X is an optional amino acid; preferably, the HEPN domain includes 1-9 RXXXXXXH and/or RXXXXXXH motifs; more preferably, the Cas13 protein includes 2, 3, 4, or 5 HEPN domains.

In a preferred embodiment, the method described in any of the preceding aspects involves selecting Cas13 proteins which HEPN domains contain the HEPN structure of the proteins listed in Table 2, or contain the HEPN structure having at least 80%, 85%, 90%, or 95% similarity to the HEPN structures of the proteins listed in Table 2.

In a preferred embodiment, the methods of any of the methods of this aspect include:
1) downloading bacterial genome and/or metagenome sequences and identify CRISPR array region;
2) analyzing proteins located upstream and downstream adjacent to the CRISPR array region, and selecting proteins whose HEPN domain contains at least one RXXXXXH and/or RXXXXXXH motif as candidate Cas13 proteins.

Preferably, the HEPN structure further contains at least one RXXXXH motif.

In a preferred embodiment, in any of the methods described in this aspect, 6 proteins located upstream and downstream of the CRISPR array region adjacent to the CRISPR array region are taken for analysis.

In a preferred embodiment, in any of the methods described in this aspect, the amino acid X adjacent to R in the HEPN structure is preferably N, Q, H or D.

In a preferred embodiment, in any of the methods described in this aspect, the protospacer flanking sequence (PFS) of candidate proteins is screened; furthermore, by assessing the PFS of candidate proteins, better functionalities of the candidate proteins are obtained.

This disclosure achieves the following technical effects:
(1) A method for rapid screening of new Cas13 protein family was developed. The method enables the analysis of CRISPR array systems of newly updated prokaryotic microbial DNA sequences and metagenomic sequences, the screening of associated effector proteins;
(2) Low molecular weight Cas13 family members are selected and the application scope of CRISPR-Cas13 is extended. Because the candidate Cas13 protein has a low molecular weight, it can be better packaged by delivery vectors such as adeno-associated virus to achieve diagnosis and treatment of related diseases, such as neurological diseases. In the field of plant biology, the candidate Cas13 protein can lead to research on breeding and stress tolerance. In microbiology, it enables the modification of relevant engineered bacteria.
(3) When using the method to screen, in addition to using the known HEPN domains of Cas13 proteins, it also includes conserved domains with RNA cleavage activity in other types of proteins. This approach provides the potential to screen for novel Cas13 proteins. Furthermore, due to the identification of these new functional domains in these new Cas13 proteins, new ideas and possibilities are provided for further modification of Cas13 proteins.

### FIGURES

FIG. 1 shows the RNase activity results of protein DZ4. The enzymatic cleavage activity of DZ4 in 293T cells is detected by flow cytometry. Co-transfecting 293T cells with plasmids containing the DZ4 protein (which also contains the sgRNA targeting mCherry) and plasmids containing the mCherry protein, followed by flow cytometry analysis 48 hours later, it is found that the candidate protein DZ4 has a strong RNase activity compared to the negative control group, and the corresponding red light is greatly knocked down. Among them, the negative control group only contains mCherry protein (red light) and DZ4 protein (green light), wherein the experiment group (also labeled as AP459) also contains one of the sgRNAs targeting a different region of mCherry.

FIG. 2 shows the RNase activity results of candidate protein DZ28. Flow cytometry experimental results for detecting cleavage activity of candidate protease in mammalian cell lines: The figure shows the results of flow cytometry analysis after co-transfecting 293T cell lines with plasmids containing the DZ28 protein (containing the sgRNA targeting mCherry) and plasmids containing the mCherry protein for 48 hours. It can be found that compared with the negative control group, the candidate protein DZ28 has strong RNase activity, and the corresponding red light is greatly knocked down. The negative control is a control group that only contains mCherry protein (red light) and DZ28 protein (green light), wherein AP393 is an experimental group containing one of the sgRNAs targeting different region of mCherry.

FIG. 3 shows the RNase activity results of protein DZ29. Flow cytometry experimental results for detecting cleavage activity of candidate protease in mammalian cell lines: The figure shows the results of flow cytometry analysis after co-transfecting 293T cell lines with plasmids containing the DZ29 protein (containing the sgRNA targeting mCherry) and plasmids containing the mCherry protein for 48 hours. It can be found that compared with the negative control group, the candidate protein DZ29 has strong RNase activity, and the corresponding red light is greatly knocked down. The negative control is a control group that only contains mCherry protein (red light) and DZ29 protein (green light), wherein control group AP405 and control group AP407 are experimental groups containing sgRNA targeting different region of mCherry.

FIG. 4 shows the flow cytometric analysis results of the candidate Cas13 protein DZ30 at the cellular level to verify its RNase activity. Flow cytometry experimental results for detecting cleavage activity of candidate protease in mammalian cell lines: The figure shows the results of flow cytometry analysis after co-transfecting 293T cell lines with plasmids containing the DZ30 protein (containing the sgRNA targeting mCherry) and plasmids containing the mCherry protein for 48 hours. It can be found that compared with the negative control group, the candidate protein DZ30 has strong RNase activity, and the corresponding red light is greatly knocked down. The negative control is a control group that only contains mCherry protein (red light) and DZ30 protein (green light), wherein AP411 and AP413 are the two experimental groups containing sgRNA targeting different region of mCherry.

FIG. 5 shows the flow cytometric analysis results of the candidate Cas13 protein DZ31 at the cellular level to verify its RNase activity. Flow cytometry experimental results for detecting cleavage activity of candidate protease in mammalian cell lines: The figure shows the results of flow cytometry analysis after co-transfecting 293T cell lines with plasmids containing the DZ31 protein (containing the sgRNA targeting mCherry) and plasmids containing the mCherry protein for 48 hours. It can be found that compared with the negative control group, the candidate protein DZ31 has strong RNase activity, and the corresponding red light is greatly knocked down. The negative control is a control group that only contains mCherry protein (red light) and DZ31 protein (green light), wherein AP417 and AP419 are the two experimental groups containing sgRNA targeting different region of mCherry.

FIG. 6 shows the flow cytometric analysis results of the candidate Cas13 protein DZ32 at the cellular level to verify its RNase activity. Flow cytometry experimental results for detecting cleavage activity of candidate protease in mammalian cell lines: The figure shows the results of flow cytometry analysis after co-transfecting 293T cell lines with plasmids containing the DZ32 protein (containing the sgRNA targeting mCherry) and plasmids containing the mCherry protein for 48 hours. It can be found that compared with the negative control group, the candidate protein DZ32 has strong RNase activity, and the corresponding red light is greatly knocked down. The negative control is a control group that only contains mCherry protein (red light) and DZ32 protein (green light), wherein AP421 and AP423 are the two experimental groups containing sgRNA targeting different region of mCherry.

FIG. 7 shows the flow cytometric analysis results of the candidate Cas13 protein DZ33 at the cellular level to verify its RNase activity. Flow cytometry experimental results for detecting cleavage activity of candidate protease in mammalian cell lines: The figure shows the results of flow cytometry analysis after co-transfecting 293T cell lines with plasmids containing the DZ33 protein (containing the sgRNA targeting mCherry) and plasmids containing the mCherry protein for 48 hours. It can be found that compared with the negative control group, the candidate protein DZ33 has strong RNase activity, and the corresponding red light is greatly knocked down. The negative control is a control group that only contains mCherry protein (red light) and DZ33 protein (green light), wherein AP427 and AP429 are the two experimental groups containing sgRNA targeting different region of mCherry.

FIG. 8 shows the flow cytometric analysis results of the candidate Cas13 protein DZ35 at the cellular level to verify its RNase activity. Flow cytometry experimental results for detecting cleavage activity of candidate protease in mammalian cell lines: The figure shows the results of flow cytometry analysis after co-transfecting 293T cell lines with plasmids containing the DZ35 protein (containing the sgRNA targeting mCherry) and plasmids containing the mCherry protein for 48 hours. It can be found that compared with the negative control group, the candidate protein DZ35 has strong RNase activity, and the corresponding red light is greatly knocked down. The negative control is a control group that only contains mCherry protein (red light) and DZ35 protein (green light), wherein AP441 and AP443 are the two experimental groups containing sgRNA targeting different region of mCherry.

FIG. 9 shows the flow cytometric analysis results of the candidate Cas13 protein DZ36 at the cellular level to verify its RNase activity. Flow cytometry experimental results for detecting cleavage activity of candidate protease in mammalian cell lines: The figure shows the results of flow cytometry analysis after co-transfecting 293T cell lines with plasmids containing the DZ36 protein (containing the sgRNA targeting mCherry) and plasmids containing the mCherry protein for 48 hours. It can be found that compared with the negative control group, the candidate protein DZ36 has strong RNase activity, and the corresponding red light is greatly knocked down. The negative control only contains mCherry protein (red light) and DZ36 protein (green light). Light) control group, wherein AP25 and AP27 are the two experimental groups containing sgRNA targeting different region of mCherry.

FIG. 10 shows the results of flow cytometry analysis of candidate Cas13 protein DZ37 RNase activity. Flow cytometric analysis experiment results for detecting cleavage activity of the candidate protease in a mammalian cell line (HEK293T). The plasmid containing DZ37 protein (containing sgRNA targeting mCherry) and the plasmid containing mCherry protein were co-transfected into the 293T cell line for 48 hours. Perform flow cytometric analysis. It can be found that compared with the negative control group, the candidate protein DZ37 targets mCherry RNA and has strong RNase activity. The corresponding red light is significantly knocked down. The negative control is a control group (without gRNA) that only contains mCherry protein (red light) and DZ37 protein (green light), wherein AP33 and AP35 are the two experimental groups containing sgRNA targeting different region of mCherry.

FIG. 11 shows the results of flow cytometry analysis of candidate Cas13 protein DZ38 RNase activity. Flow cytometric analysis experiment results for detecting cleavage activity of the candidate protease in a mammalian cell line (HEK293T). The plasmid containing DZ38 protein (containing sgRNA targeting mCherry) and the plasmid containing mCherry protein were co-transfected into the 293T cell line for 48 hours. Perform flow cytometric analysis. It can be found that compared with the negative control group, the candidate protein DZ38 targets mCherry RNA and has strong RNase activity. The corresponding red light is significantly knocked down. The negative control is a control group (without gRNA) that only contains mCherry protein (FB132) and DZ38 protein (green light), wherein AP38 and AP47 are the two experimental groups containing sgRNA targeting different region of mCherry.

FIG. 12 shows flow cytometry analysis results of RNase activity of the candidate Cas13 protein DZ39. Flow cytometric analysis experiment results for detecting cleavage activity of candidate protease in a mammalian cell line (HEK293T). The plasmid containing DZ39 protein (containing sgRNA targeting mCherry) and the plasmid containing mCherry protein were co-transfected into the 293T cell line for 48 hours. Perform flow cytometric analysis. It can be found that compared with the negative control group, the candidate protein DZ39 targets mCherry RNA and has a certain RNase activity. The corresponding red fluorescence is slightly knocked down. The negative control is a control group (without gRNA) that only contains mCherry protein (FB132) and DZ39 protein (green light), wherein AP39 and AP43 are the two experimental groups containing sgRNA targeting different region of mCherry.

FIG. 13 shows the flow cytometry analysis results of the RNase activity of candidate Cas13 protein DZ40. Flow cytometric analysis experiment results for detecting cleavage activity of the candidate protease in a mammalian cell line (HEK293T). The plasmid containing DZ40 protein (containing sgRNA targeting mCherry) and the plasmid containing mCherry protein were co-transfected into the 293T cell line for 48 hours. Perform flow cytometric analysis. It can be found that compared with the negative control group, the candidate protein DZ40 targets mCherry RNA and has a certain RNase activity. The corresponding red light is knocked down. The negative control is a control group (without gRNA) that only contains mCherry protein (FB132) and DZ40 protein (green light), wherein AP49 and AP53 are the two experimental groups containing sgRNA targeting different region of mCherry.

FIG. 14 shows the flow cytometry analysis results of the RNase activity of candidate Cas13 protein DZ44. Flow cytometric analysis experiment results for detecting cleavage activity of detect the candidate protease in a mammalian cell line (HEK293T). The plasmid containing DZ44 protein (containing sgRNA targeting mCherry) and the plasmid containing mCherry protein were co-transfected into the 293T cell line for 48 hours. Perform flow cytometric analysis. It can be found that compared with the negative control group, the candidate protein DZ44 targets mCherry RNA and has a certain RNase activity. The corresponding red light is knocked down. The negative control is a control group (without gRNA) that only contains mCherry protein (FB132) and DZ44 protein (green light), wherein AP59 and AP55 are the two experimental groups containing sgRNA targeting different region of mCherry.

FIG. 15 shows the flow cytometry analysis results of the RNase activity of candidate Cas13 protein DZ45. Flow cytometric analysis experiment results for detecting cleavage activity of candidate protease in a mammalian cell line (HEK293T). The plasmid containing DZ45 protein (containing sgRNA targeting mCherry) and the plasmid containing mCherry protein were co-transfected into the 293T cell line for 48 hours. Perform flow cytometric analysis. It can be found that compared with the negative control group, the candidate protein DZ45 targets mCherry RNA and has strong RNase activity. The corresponding red light is knocked down significantly. The negative control is a control group (without gRNA) that only contains mCherry protein (FB132) and DZ45 protein (green light), wherein AP63 and AP65 the two are experimental groups containing sgRNA targeting different region of mCherry.

FIG. 16 shows the flow cytometry analysis results of the RNase activity of candidate Cas13 protein DZ46. Flow cytometric analysis experiment results for detecting cleavage activity of the candidate protease in a mammalian cell line (HEK293T). The plasmid containing DZ46 protein (containing sgRNA targeting mCherry) and the plasmid containing mCherry protein were co-transfected into the 293T cell line for 48 hours. Perform flow cytometric analysis. It can be found that compared with the negative control group, the candidate protein DZ46 targets mCherry RNA and has strong RNase activity. The corresponding red light is knocked down significantly. The negative control is a control group (without gRNA) that only contains mCherry protein (FB132) and DZ46 protein (green light), wherein AP69 and AP71 are the two experimental groups containing sgRNA targeting different region of mCherry.

FIG. 17 shows the flow cytometry analysis results of the RNase activity of candidate Cas13 protein DZ47. Flow cytometric analysis experiment results for detecting cleavage activity of the candidate protease in a mammalian cell line (HEK293T). The plasmid containing DZ47 protein (containing sgRNA targeting mCherry) and the plasmid containing mCherry protein were co-transfected into the 293T cell line for 48 hours. Perform flow cytometric analysis. It can be found that compared with the negative control group, the candidate protein DZ47 targets mCherry RNA and has strong RNase activity. The corresponding red light is knocked down significantly. The negative control is a control group (without gRNA) that only contains mCherry protein (FB132) and DZ47 protein (green light), wherein AP91 and AP93 are the two experimental groups containing sgRNA targeting different region of mCherry.

FIG. 18 shows the flow cytometry analysis results of the RNase activity of candidate Cas13 protein DZ50. Flow cytometric analysis experiment results for detecting cleavage activity of the candidate protease in a mammalian cell line (HEK293T). The plasmid containing DZ50 protein (containing sgRNA targeting mCherry) and the plasmid containing mCherry protein were co-transfected into the 293T cell line for 48 hours. Perform flow cytometric analysis. It can be found that compared with the negative control group, the candidate protein DZ50 targets mCherry RNA and has a certain RNase activity. The corresponding red light is knocked down. The negative control is a control group (without gRNA) that only contains mCherry protein (FB132) and DZ50 protein (green light), wherein AP121 and AP125 are the two experimental groups containing sgRNA targeting different region of mCherry.

FIG. 19 shows the flow cytometry analysis results of the RNase activity of candidate Cas13 protein DZ51. Flow cytometric analysis experiment results for detecting cleavage activity of the candidate protease in a mammalian cell line (HEK293T). The plasmid containing DZ51 protein (containing sgRNA to targeting mCherry) and the plasmid containing mCherry protein were co-transfected into the 293T cell line for 48 hours and then analyzed by flow cytometry. It can be found that compared with the negative control group, the candidate protein DZ51 targets mCherry RNA and has strong RNase activity. The corresponding red light is knocked down significantly. The negative control is a control group (without gRNA) that only contains mCherry protein (FB132) and DZ51 protein (green light), wherein AP127 and AP131 are the two experimental groups containing sgRNA targeting different region of mCherry.

FIG. 20 shows the flow cytometry analysis results of the RNase activity of candidate Cas13 protein DZ52. Flow cytometric analysis experiment results for detecting cleavage activity of the candidate protease in a mammalian cell line (HEK293T). The plasmid containing DZ52 protein (containing gRNA targeting mCherry) and the plasmid containing mCherry protein were co-transfected into the 293T cell line for 48 hours and then analyzed by flow cytometry. It can be found that compared with the negative control group, the candidate protein DZ52 can target mCherry RNA and has strong RNase activity. The corresponding red light is knocked down. The negative control is a control group (without gRNA) that only contains mCherry protein (FB132) and DZ52 protein (green light), wherein AP133 and AP135 are experimental the two groups containing gRNA targeting different region of mCherry.

FIG. 21 shows the flow cytometry analysis results of the RNase activity of candidate Cas13 protein DZ54. Flow cytometric analysis experiment results for detecting cleavage activity of the candidate protease in a mammalian cell line (HEK293T). The plasmid containing DZ54 protein (containing sgRNA targeting mCherry) and the plasmid containing mCherry protein were co-transfected into the 293T cell line for 48 hours and then analyzed by flow cytometry. It can be found that compared with the negative control group, the candidate protein DZ54 targets mCherry RNA and has a certain RNase activity. The corresponding red light is knocked down. The negative control is a control group (without gRNA) that only contains mCherry protein (FB132) and DZ54 protein (green light), wherein AP153 is an experimental group containing gRNA targeting mCherry.

FIG. 22 shows the flow cytometry analysis results of the RNase activity of candidate Cas13 protein DZ55. Flow cytometric analysis experiment results for detecting cleavage activity of the candidate protease in a mammalian cell line (HEK293T). The plasmid containing DZ55 protein (containing sgRNA targeting mCherry) and the plasmid containing mCherry protein were co-transfected into the 293T cell line for 48 hours and then analyzed by flow cytometry. It can be found that compared with the negative control group, the candidate protein DZ55 targets mCherry RNA and has a certain RNase activity. The corresponding red light is knocked down. The negative control is a control group (without gRNA) that only contains mCherry protein (FB132) and DZ55 protein (green light), wherein AP157 is an experimental group containing gRNA targeting mCherry.

FIG. 23 shows the flow cytometry analysis results of the RNase activity of candidate Cas13 protein DZ57. Flow cytometric analysis experiment results for detecting cleavage activity of candidate protease in a mammalian cell line (HEK293T). The plasmid containing DZ57 protein (containing gRNA targeting mCherry) and the plasmid containing mCherry protein were co-transfected into the 293T cell line for 48 hours and then analyzed by flow cytometry. It can be found that compared with the negative control group, the candidate protein DZ57 targets mCherry RNA and has a certain RNase activity. The corresponding red light is knocked down. The negative control is a control group (without gRNA) that only contains mCherry protein (FB132) and DZ57 protein (green light), wherein AP169 and AP171 are the two experimental groups containing gRNAs targeting different region of mCherry.

FIG. 24 shows the flow cytometry analysis results of the RNase activity of candidate Cas13 protein DZ62. Flow cytometric analysis experiment results for detecting cleavage activity of candidate protease in a mammalian cell line (HEK293T). The plasmid containing DZ62 protein (containing gRNA targeting mCherry) and the plasmid containing mCherry protein were co-transfected into the 293T cell line for 48 hours and then analyzed by flow cytometry. It can be found that compared with the negative control group, the candidate protein DZ62 targets mCherry RNA and has strong RNase activity. The corresponding red light is knocked down significantly. The negative control is a control group (without gRNA) that only contains mCherry protein (FB132) and DZ62 protein (green light), wherein AP187 and AP191 are the two experimental groups containing gRNAs targeting different region of mCherry.

FIG. 25 shows the flow cytometry analysis results of the RNase activity of candidate Cas13 protein DZ63. Flow cytometric analysis experiment results for detecting cleavage activity of candidate protease in a mammalian cell line (HEK293T). The plasmid containing DZ63 protein (containing gRNA targeting mCherry) and the plasmid containing mCherry protein were co-transfected into the 293T cell line for 48 hours and then analyzed by flow cytometry. It can be found that compared with the negative control group, the candidate protein DZ63 targets mCherry RNA and has strong RNase activity. The corresponding red light is knocked down significantly. The negative control is a control group (without gRNA) that only contains mCherry protein (FB132) and DZ63 protein (green light), wherein AP193 and AP197 are the two experimental groups containing gRNAs targeting different region of mCherry.

FIG. 26 shows the flow cytometry analysis results of the RNase activity of candidate Cas13 protein DZ65. Flow cytometric analysis experiment results for detecting cleavage activity of candidate protease in a mammalian cell line (HEK293T). The plasmid containing DZ65 protein (containing gRNA targeting mCherry) and the plasmid containing mCherry protein were co-transfected into the 293T cell line for 48 hours and then analyzed by flow cytometry. It can be found that compared with the negative control group, the candidate protein DZ65 can target mCherry RNA and has strong RNase activity. The corresponding red light is knocked down significantly. The negative control is a control group (without gRNA) that only contains mCherry protein (FB132) and DZ65 protein (green light), wherein AP201 and AP203 are the two experimental groups containing gRNAs targeting different region of mCherry.

FIG. 27 shows the flow cytometry analysis results of the RNase activity of candidate Cas13 protein DZ68. Flow cytometric analysis experiment results for detecting cleavage activity of candidate protease in a mammalian cell line (HEK293T). The plasmid containing DZ68 protein (containing gRNA targeting mCherry) and the plasmid containing mCherry protein were co-transfected into the 293T cell line for 48 hours and then analyzed by flow cytometry. It can be found that compared with the negative control group, the candidate protein DZ68 targets mCherry RNA and has strong RNase activity. The corresponding red light is knocked down significantly. The negative control is a control group (without gRNA) that only contains mCherry protein (FB132) and DZ68 protein (green light), wherein AP217 and AP219 are the two experimental groups containing gRNAs targeting different region of mCherry.

FIG. 28 shows the flow cytometry analysis results of the RNase activity of candidate Cas13 protein DZ86. Flow cytometric analysis experiment results for detecting cleavage activity of the candidate protease in a mammalian cell line (HEK293T). The plasmid containing DZ86 protein (containing gRNA targeting mCherry) and the plasmid containing mCherry protein were co-transfected into the 293T cell line for 48 hours and then analyzed by flow cytometry. It can be found that compared with the negative control group, the candidate protein DZ86 targets mCherry RNA and has strong RNase activity. The corresponding red light is knocked down significantly. The negative control is a control group (without gRNA) that only contains mCherry protein (FB132) and DZ86 protein (green light), wherein AP711 and AP713 are the two experimental groups containing gRNA targeting different region of mCherry.

FIG. 29 shows the flow cytometric analysis results of the candidate Cas13 protein DZ90 at the cellular level to verify its RNase activity. Flow cytometry experimental results for detecting candidate protease cleavage activity in mammalian cell lines: The figure shows the results of flow cytometry analysis after co-transfecting 293T cell lines with plasmids containing the DZ90 protein (containing the sgRNA targeting mCherry) and plasmids containing the mCherry protein for 48 hours. It can be found that compared with the negative control group, the candidate protein DZ90 has strong RNase activity, and the corresponding red light is greatly knocked down. The negative control is a control group that only contains mCherry protein (red light) and DZ90 protein (green light), wherein AP313 and AP317 are the two experimental groups containing sgRNAs targeting different region of mCherry.

FIG. 30 shows the flow cytometric analysis results of the candidate Cas13 protein DZ91 at the cellular level to verify its RNase activity. Flow cytometry experimental results for detecting candidate protease cleavage activity in mammalian cell lines: The figure shows the results of flow cytometry analysis after co-transfecting 293T cell lines with plasmids containing the DZ91 protein (containing the sgRNA targeting mCherry) and plasmids containing the mCherry protein for 48 hours. It can be found that compared with the negative control group, the candidate protein DZ91 has strong RNase activity, and the corresponding red light is greatly knocked down. The negative control is a control group that only contains mCherry protein (red light) and DZ91 protein (green light), wherein AP319 and AP323 are the two experimental groups containing sgRNA targeting different region of mCherry.

FIG. 31 shows the flow cytometric analysis results of the candidate Cas13 protein DZ93 at the cellular level to verify its RNase activity. Flow cytometry experimental results for detecting candidate protease cleavage activity in mammalian cell lines: The figure shows the results of flow cytometry analysis after co-transfecting 293T cell lines with plasmids containing the DZ93 protein (containing the sgRNA targeting mCherry) and plasmids containing the mCherry protein for 48 hours. It can be found that compared with the negative control group, the candidate protein DZ93 has strong RNase activity and the corresponding red light is knocked down in a certain extent. The negative control is a control group that only contains mCherry protein (red light) and DZ93 protein (green light), wherein AP151 is an experimental group containing one of the sgRNAs targeting different region of mCherry.

FIG. 32 shows the flow cytometric analysis results of the candidate Cas13 protein DZ96 at the cellular level to verify its RNase activity. Flow cytometry experimental results for detecting candidate protease cleavage activity in mammalian cell lines: The figure shows the results of flow cytometry analysis after co-transfecting 293T cell lines with plasmids containing the DZ96 protein (containing the sgRNA targeting mCherry) and plasmids containing the mCherry protein for 48 hours. It can be found that compared with the negative control group, the candidate protein DZ96 has strong RNase activity, and the corresponding red light is greatly knocked down. The negative control is a control group that only contains mCherry protein (red light) and DZ96 protein (green light), wherein AP349 and AP353 are the two experimental groups containing sgRNA targeting different region of mCherry.

FIG. 33 shows the flow cytometric analysis results of the candidate Cas13 protein DZ98 at the cellular level to verify its RNase activity. Flow cytometry experimental results for detecting candidate protease cleavage activity in mammalian cell lines: The figure shows the results of flow cytometry analysis after co-transfecting 293T cell lines with plasmids containing the DZ98 protein (containing the sgRNA targeting mCherry) and plasmids containing the mCherry protein for 48 hours. It can be found that compared with the negative control group, the candidate protein DZ98 has strong RNase activity, and the corresponding red light is greatly knocked down. The negative control is a control group that only contains mCherry protein (red light) and DZ98 protein (green light), wherein AP361 is the experimental group containing one of the sgRNAs targeting different region of mCherry.

FIG. 34 shows the flow cytometric analysis results of the positive control Cas13d protein to verify its RNase activity at the cellular level. Flow cytometry experiment results for detecting candidate protease cleavage activity in mammalian cell lines: The figure shows the results of flow cytometry analysis after co-transfecting 293T cell lines with plasmids containing the Cas13d protein (containing the sgRNA targeting mCherry) and plasmids containing the mCherry protein for 48 hours. It can be found that compared with the negative control group, the candidate protein Cas13d has strong RNase activity, and the corresponding red light is greatly knocked down. The negative control is a control group that only contains mCherry protein (red light) and Cas13d protein (green light), wherein px261 is an experimental group containing one of the sgRNAs targeting different region of mCherry.

FIG. 35 shows the qPCR results of the candidate protein DZ4 knocking down the endogenous gene STAT3. It can be found that the endogenous gene can be knocked down in a certain extent if sgRNA randomly designed to target STAT3 transiently transfects with the DZ4 protein.

FIG. 36A and FIG. 36B show the qPCR results of the candidate protein DZ29 knocking down the endogenous genes STAT3 and EZH2. It can be found that the endogenous genes can be knocked down in a certain extent if sgRNA randomly designed to target STAT3 or EZH2 transiently transfects with the DZ29. Wherein different DRs have a certain impact on the ability of DZ29 to knock down endogenous genes.

FIG. 37 shows the qPCR results of the candidate protein DZ32 knocking down the endogenous gene EZH2. It can be found that the endogenous gene can be knocked down in a certain extent if sgRNA randomly designed to target EZH2 transiently transfects with the DZ32 protein.

FIG. 38 shows the qPCR results of the candidate protein DZ47 knocking down the endogenous genes STAT3 and EZH2. It can be found that the endogenous genes can be knocked down in a certain extent if sgRNA randomly designed to target STAT3 or EZH2 transiently transfects with the DZ47.

FIG. 39 shows the qPCR results of the candidate protein DZ51 knocking down the endogenous genes STAT3 and EZH2. It can be found that the endogenous gene can be knocked down in a certain extent if sgRNA randomly designed to target STAT3 transiently transfects with the DZ51 protein.

FIG. 40 shows the qPCR results of the candidate protein DZ54 knocking down the endogenous genes STAT3 and EZH2. It can be found that the endogenous genes can be knocked down in a certain extent if sgRNA randomly designed to target STAT3 or EZH2 transiently transfects with the DZ54.

FIG. 41 shows the qPCR results of the candidate protein DZ68 knocking down the endogenous genes STAT3 and EZH2. It can be found that the endogenous gene can be knocked down in a certain extent if sgRNA randomly designed to target STAT3 or EZH2 transiently transfects with the DZ68 protein.

FIG. 42A and FIG. 42B show the qPCR results of the candidate protein DZ93 knocking down the endogenous genes STAT3 and EZH2. It can be found that the endogenous genes can be knocked down in a certain extent if sgRNA randomly designed to target STAT3 or EZH2 transiently transfects with the DZ93. Wherein different DRs have a certain impact on the ability of DZ93 to knock down endogenous genes.

FIG. 43 shows the qPCR results of candidate protein DZ98 knocking down the endogenous gene STAT3. It can be found that the endogenous gene can be knocked down in a certain extent if sgRNA randomly designed to target STAT3 transiently transfects with the DZ98 protein.

FIG. 44 shows the qPCR results of the candidate protein knocking down the 293T endogenous gene STAT3. The boxed part shows part of the protein that has the potential efficiency to knock down RNase of STAT3 compared to the control group.

FIG. 45A and FIG. 45B show the qPCR results of the candidate protein DZ806 knocking down the endogenous genes STAT3 and EZH2. It can be found that the endogenous genes can be knocked down in a certain extent if sgRNA randomly designed to target STAT3 or EZH2 transiently transfects with the DZ806.

FIG. 46A and FIG. 46B show the qPCR results of the candidate protein DZ821 knocking down the endogenous genes STAT3 and EZH2. It can be found that the endogenous genes can be knocked down in a certain extent if sgRNA randomly designed to target STAT3 or EZH2 transiently transfects with the DZ821.

FIG. 47A and FIG. 47B show the qPCR results of the candidate protein DZ822 knocking down the endogenous genes STAT3 and EZH2. It can be found that the endogenous genes can be knocked down in a certain extent if sgRNA randomly designed to target STAT3 or EZH2 transiently transfects with the DZ822.

FIG. 48A and FIG. 48B show the qPCR results of the candidate protein DZ825 knocking down the endogenous genes STAT3 and EZH2. It can be found that the endogenous genes can be knocked down in a certain extent if sgRNA randomly designed to target STAT3 or EZH2 transiently transfects with the DZ825.

FIG. 49A shows the experimental results of the preferred motif analysis for RNA targeting and cleavage by DZ796. It can be found that it has a strong base preference at 5′. The first base adjacent to the 5′ end of the target sequence is G or C, while adjacent to the 3′ end is G; the PFS of 3′ is not obvious. The overall PFS is 5′ [C/G]-targetSeq-NNNNN[G]-3′.

FIG. 49B shows the experimental results of the preferred motif analysis for RNA targeting and cleavage by DZ806. It can be found that it has strong base preference at both 5′ and 3′. The third base adjacent to the 5′ end of the target sequence has a strong T preference, while the first and second bases are mainly G or A preference. The first base adjacent to the 3′ end shows T preference. The overall PFS is 5′-T[G/A][G/A]-targetSeq-TN[G/A]-3′.

FIG. 49C shows the experimental results of the preferred motif analysis for RNA targeting and cleavage by DZ821. It can be found that it has relatively strong base preference at both 5′ and 3′. The 5th base adjacent to the 5′ end and 3′ end of the target sequence has a strong T preference. The overall PFS is 5′-TNNN[C/G]-targetSeq-NNNNT-3′.

FIG. 49D shows the experimental results of the preferred motif analysis for RNA targeting and cleavage byDZ822. It can be found that it has a relatively strong base preference at 5′. The third base adjacent to the 5′ end of the target sequence has a strong T preference, while the 3′ end has a weaker PFS and the third base of the target sequence has G or C preference. The overall PFS is 5′-TN[G/C/A]-targetSeq-[G/A][C/G][G/C]-3′

FIG. 49E shows the experimental results of the preferred motif analysis for RNA targeting and cleavage byDZ824. It can be found that it has a relatively strong base preference at 5′. The third base adjacent to the 5′ end of the target sequence has a strong T preference, while the 3′ end has a weaker PFS and the second base adjacent to the target sequence has a weak G preference. The overall PFS is 5′-N[C/G][C/G][C/T]-targetSeq-NG[C/A]-3′.

FIG. 49F shows the experimental results of the preferred motif analysis for RNA targeting and cleavage byDZ825. It can be found that it has strong base preference at both 5′ and 3′. The first base adjacent to the 5′ end of the target sequence is C, while adjacent to the 3′ end is G.

FIG. 50A shows the ability of the PFS of DZ825 to knock down (KD) the endogenous gene. Two endogenous genes STAT3 and EZH2 of 293T were chosen. The first group in each gene experimental group uses a spacer designed without prior knowledge of the PFS of DZ825, while the subsequent three groups use the newly designed spacers based on the PFS motif of DZ825. As observed in the figure, some of the newly designed sgRNAs demonstrate better knockdown efficiency in the 293T cell lines of the knockdown experiment.

FIG. 50B shows the ability of the PFS of DZ822 PFS to knock down (KD) the endogenous gene. Three endogenous genes STAT3, EGFR and HRAS of 293T were chosen. The first one in each gene experimental group uses a spacer designed without prior knowledge of the PFS, while the subsequent groups use the newly designed spacers based on the PFS motif. As observed in the figure, some of the newly designed sgRNAs, such as KRAS, demonstrate better knockdown efficiency in the 293T cell lines of the knockdown experiment.

FIG. 50C shows the ability of the PFS of DZ806 PFS to knock down (KD) endogenous genes in 293T cells. The selected endogenous genes include STAT3, EZH2, EGFR, HRAS, RAF1, NF2, SMARCA4, NFKB1, PPARG, KRAS, PTBP1 and NRAS. The first one in each gene knockdown experimental group use a spacer designed without prior knowledge of the PFS, while the subsequent groups use the newly designed spacers based on the PFS motif. As observed in the figure, some of the newly designed sgRNAs, such as NF2 and SMARCA4, demonstrate better knockdown efficiency in the 293T cell lines of in the knockdown experiment.

FIG. 50D shows the optimal effect of knocking down endogenous genes in 293T cells using the original protein DZ806. Among the genes tested so far, the one with the highest knockdown efficiency is the KD EGFR gene, which exceeds 50%;

FIG. 51 shows the evolutionary relationship between the candidate CRISPR-Cas13 with guide RNA and potential RNase activity and the known Cas13 protein family members. It can be found that our candidate protein is potentially divided into two new families. They are named Cas13 ml and Cas13m2 temporary, such as DZ30, DZ32; DZ47, DZ29 of the Cas13m2 family, etc.

### DETAILS

The following will provide a detailed description of the embodiments of the present invention in conjunction with examples. It should be understood that the following examples are only used to illustrate the present invention and should not be considered as limiting the scope of the present invention. If the specific conditions are not specified in the examples, the conditions should be carried out according to the conventional conditions or the conditions recommended by the manufacturer.

Unless defined otherwise, all technical and scientific terms used in this application have the same meaning as commonly understood by the ordinary skilled person in the art of the present invention. Unless otherwise indicated, the present invention is practiced using conventional methods of chemistry, biochemistry, biophysics, molecular biology, cell biology, genetics, immunology and pharmacology known to the skilled person in the art.

It should be noted that all headings and subheadings used in this application are for convenience only and should not be explained as limiting the invention in any way.

Unless defined otherwise, the use of exemplary wording (eg, “such as”) provided in this application is intended to be illustrative only and is not intended to limit the scope of the invention.

As used herein, “a” or “an” or “the” may mean one or more than one. Unless defined otherwise in this specification, the terms presented in singular form also include the plural form.

In this text, a noun without a quantifier may mean one or more. As used in the claims, when used in conjunction with the word “comprise/include”, a noun without a quantifier may mean one or more than one.

In this text, the term “or” is used to mean “and/or”, regardless of whether the content in this text only adopts the alternative options or adopts both “and” and “or” options, unless otherwise specified or the alternatives are completely independent.

In the text, “another” may refer to at least another one or more.

In the text, the term “about” is used to indicate the error of a value. Such error may be a variation of ±10% from the stated value.

In the text, unless otherwise stated, nucleotide sequences are listed in the 5′ to 3′ orientation and amino acid sequences are listed in the N-terminal to C-terminal orientation.

### Definition

NCBI (https://www.ncbi.nlm.nih.gov/) refers to the U.S. National Center for Biological Information. It is a public database for the world. Those skilled in the field use the nucleic acid database provided by this database to download prokaryotes to download the prokaryotic genome and proteome-related databases, etc. It analysis the sequence by BLAST alignment software provided by the database.

IMG (https://img.jgi.doe.gov/) refers to the Integrated Microbial Genome Database and is a representative of new generation genome databases. It can not only completely include the contents of existing databases, but also provide more complete services of data upload, annotation, and analysis, as well as store the sequencing data in IMG/M database. This database can be used to download the sequencing genome of pure culture bacterial sequencing genomes, metagenomes, metagenome-assembled genomes, and single-cell sequencing genomes.

The term “CRISPR” (cluster regularly interspaced short palindromic repeats) refers to a DNA sequence in the prokaryotic genome, including a direct repeat (DR) region and a non-repeating spacer region.

The term “CRISPR array” refers to the region containing repeat sequences and spacer sequences.

The term “CRISPR-Cas system” refers to a system containing a CRISPR array and associated Cas proteins.

The Cas13 family is a family of CRISPR enzymes that can target RNA. Its members include Cas13a, Cas13b, Cas13c, Cas13d, Cas13X and Cas13Y families. Unlike CRISPR/Cas9, which cuts DNA, CRISPR/Cas13 can be used to cut specific RNA sequences in bacterial cells.

The term “HEPN domain” is the abbreviation of higher eukaryotes and prokaryotes nucleotide domain. It is an important domain of the Cas13 protein in the CRISPR-Cas13 enzyme system which enable the cleavage and defense against foreign invading nucleic acids.

The term “ABE system” is the abbreviation of Adenine base editors, which is a purine base conversion technology that can achieve single base changes from A/T to G/C. The most commonly used enzyme is adar enzyme (adenosine deaminases acting on RNA, an adenosine deaminase acting on RNA). It can deaminate adenosine into inosine, which would be seen as G when read in DNA or RNA, thus achieving the mutation from A/T to G/C. This mutation maintains high product purity because cells are insensitive to inosine excision repair.

The term “CBE system” is the abbreviation of Cytidine base editor, which is pyrimidine base conversion technology. The current tools include BE1, BE2 and BE3. Among them, BE3 has the highest efficiency and therefore it is used widely in the fields of gene therapy, animal model production, and functional gene screening.

The term “eukaryotic cell” is, for example, a mammalian cell, including human cells (human primary cells or the established human cell lines). The cells may be non-human mammalian cells, for example from non-human primates (e.g. monkeys), cows/bulls/cattle, sheep, goats, pigs, horses, dogs, cats, rodents (e.g. rabbits, rats, hamsters), etc. The cells are from fish (eg, salmon), birds (e.g., poultry, including chickens, ducks, geese), reptiles, shellfish (e.g., oysters, clams, lobsters, shrimp), insects, worms, yeast, and the like. The cells may be from plants, such as monocots or dicots. The plant may be a food crop such as barley, cassava, cotton, peanut, corn, millet, oil palm, potato, legume, rapeseed or canola, rice, rye, sorghum, soybean, sugarcane, sugar beet, sunflower and wheat. The plant may be a cereal (e.g. barley, corn, millet, rice, rye, sorghum and wheat). The plants may be tubers (e.g. cassava and potatoes). In some embodiments, the plant may be a sugar crop (e.g., sugar beet and sugar cane). The plants may be oily crops (e.g. soybeans, peanuts, rapeseed or canola, sunflowers and oil palm fruits). The plant may be a fiber crop (e.g. cotton). The plant may be a tree such as a peach or nectarine tree, an apple tree, a pear tree, an apricot tree, a walnut tree, a pistachio tree, a citrus tree (e.g. orange, grapefruit, or lemon tree), grass, vegetable, fruit, or algae. The plant may be a plant of Solanum; Brassica; Lactuca; Spinacia; Capsicum; cotton, tobacco, asparagus, carrot, cabbage, broccoli, cauliflower, tomatoes, eggplants, peppers, lettuce, spinach, strawberries, blueberries, raspberries, blackberries, grapes, coffee, cocoa, etc.

The term “host cell” in this application includes any cells that express the cas13 protein described in this application, or the nucleic acid molecule transduced with the cas13 protein, or the CRISPR-Cas system, or the delivery system, including prokaryotic cells and eukaryotic cells.

### CRISPR System

CRISPR (Clustered Regularly Interspaced Short Palindromic Repeats)/Cas13 (CRISPR-associated protein 13)-mediated RNA editing is becoming a promising tool for disease diagnosis and treatment, plant breeding, etc.

CRISPR is a DNA locus that contains short repeats of a base sequence. Each repeat is followed by a short segment of “spacer DNA” which previous exposure to the virus. CRISPR is found in approximately 40% of sequenced bacterial genomes and 90% of sequenced archaea. CRISPR is often associated with Cas genes that encode CRISPR-related proteins. The CRISPR/Cas system is a prokaryotic immune system that confers resistance to foreign genetic elements such as plasmids and phages and provides a form of acquired immunity. CRISPR spacers recognize and silence these foreign genetic elements (e.g., RNAi) in eukaryotic organisms.

The size of CRISPR repeats has 24 to 48 base pairs. They usually exhibit some dyad symmetry, which suggests the formation of secondary structures such as hairpins, rather than true palindromes palindromic structures. Repeated sequences are separated by spacer sequences of similar lengths. Some CRISPR spacer sequences match exactly with sequences derived from plasmids and phages, although some spacers also match with the genomes of prokaryotes. New spacers can be rapidly added in response to phage infection.

The “guide RNA (gRNA)” is a sequence in the guide RNA that is complementary (partially complementary or completely complementary) and/or hybridizes with the target sequence in the target nucleic acid, thereby enabling the CRISPR-CAS complex (such as CRISPR-Cas13 complex) is guided and specifically bounden to the target nucleic acid sequence.

### Nuclease

In this application, “Cas nuclease” and “cas13 protein” are used interchangeably. CRISPR-associated (Cas) genes are often associated with CRISPR repeat-spacer arrays. As of 2013, more than forty different families of Cas proteins have been described. Among these protein families, Cas1 appears to be ubiquitous in different CRISPR/Cas systems. Specific combinations of Cas genes and repeat structures have been used to define eight CRISPR subtypes (E coli, Ypest, Nmeni, Dvulg, Tneap, Hmari, Apern, and Mtube), some of which are associated with other gene modules encoding repeat-associated mysterious proteins (RAMP). More than one CRISPR subtype can exist in a single genome. The sporadic distribution of CRISPR/Cas subtypes suggests that this system has undergone horizontal gene transfer during microbial evolution.

The foreign DNA is apparently processed into small elements (about 30 base pairs in length) by the proteins encoded by the Cas genes, which are then somehow inserted into the CRISPR locus near to the leader sequence. RNA from the CRISPR locus is constitutively expressed and processed by Cas proteins into small RNAs composed of individual exogenous sequence elements with flanking repeats. RNA directs other Cas proteins to silence foreign genetic elements at the RNA or DNA level. Evidence shows functional diversity among CRISPR subtypes. The Cse (Cas subtype E coli) protein (called as CasA-E in Escherichia coli (E. coli)) forms a functional complex Cascade, which processes CRISPR RNA transcripts into spacer-repeat sequence units that retain Cascade. In other prokaryotes, Cas6 processes CRISPR transcripts. Interestingly, CRISPR-based phage inactivation in E. coli requires Cascade and Cas3, but not Cas1 and Cas2. The Cmr (Cas RAMP module) protein found in Pyrococcus furiosus and other prokaryotes forms a functional complex with small CRISPR RNA, which recognizes and cleaves complementary target RNA. RNA-guided CRISPR enzymes are classified as type V restriction enzymes.

The following specific examples are provided to further illustrate the content of the present invention. It should be understood that these examples are merely illustrative of the disclosure and are not intended to limit the scope of the disclosure. Experimental methods without specifying specific conditions in the following examples usually are generally performed conventional conditions, such as those described in Sambrook et al., Molecular Cloning: Laboratory Manual (New York: Cold Spring Harbor Laboratory Press, 1989), or according to the conditions recommended by the manufacturer. Unless otherwise stated, percentages and parts are by weight.

Unless otherwise stated, the materials and reagents used in the examples of this disclosure are commercially available products.

### EXAMPLES

### Example 1: Screening of New Cas13 Proteins

It is generally believed in the art that the HEPN domain necessary for Cas13 function refers to the sequence of RxxxxH (R4xH). Therefore, in the process of screening potential Cas13, the presence of at least two R4xH domains is typically used as the first screening criterion. However, the applicant found that RxxxxxH (R5xH) or RxxxxxxH (R6xH) also can serve as the HEPN structure of Cas13 domain comes into play. Therefore, the inventors used R4xH, R5xH and R6xH (hereinafter referred to as extended HEPN domains) as screening criterion during the screening process, leading to the discovery of a class of cas13 proteins with new HEPN domains (R5xH and R6xH). The inventors also found that the molecular weight of these cas13 proteins containing R5xH and R6xH type HEPN domains is much smaller than that of known cas13. This means that the R5xH and R6xH domains are likely to be characteristic structures of a class of smaller molecular weight Cas13 proteins, thus provide a method for screening smaller cas13 proteins.

We first download the sequences of all bacterial, archaeal genomes and metagenomes from NCBI and IMG as of July 2021, then use CRISPR array identification software (such as Pilercr) to identify the CRISPR array region. 78 candidate proteins are obtained through target domain analysis on six proteins located upstream and downstream adjacent to the CRISPR array region. The information of the extended HEPN domains and coordinates of the candidate proteins are shown in Table 2.

The HEPN domain of the candidate cas13 protein contains the RxxxxH (R4xH) motif, RxxxxxH (R5xH) motifs, and RxxxxxxH (R6xH) motifs, wherein x represents any amino acid. The conserved amino acid adjacent to R is preferably to be N, Q, H or D, such as R[NDQH]xxxH, R[NDQH]xxxxH, R[NDQH]xxxxxH and other combinations. R4xH, R5xH and R6xH are respectively preferably to be R[NDQH]xxxH, R[NDQH]xxxxH, and R[NDQH]xxxxxH.

### Example 2: Verification of RNA Enzyme Cleavage Activity

The nucleic acid sequence, DR sequence and target spacer sequence of the candidate protein are synthesized, and then introduced into the expression plasmids to construct the corresponding plasmids. The plasmids are transformed into DH-5a E. coli competent cells for amplification and culture. The plasmids are extracted and then transfected the human 293T cell lines (capable of expressing red light). Negative and positive control groups are designed. The negative control only contains mCherry protein (recorded as FB132), and the positive control is the cas13d protein. After 48 hours of co-transfection with the plasmids, flow cytometry analysis and other experiments are conducted to determine the RNA cleavage activity of the candidate protein.

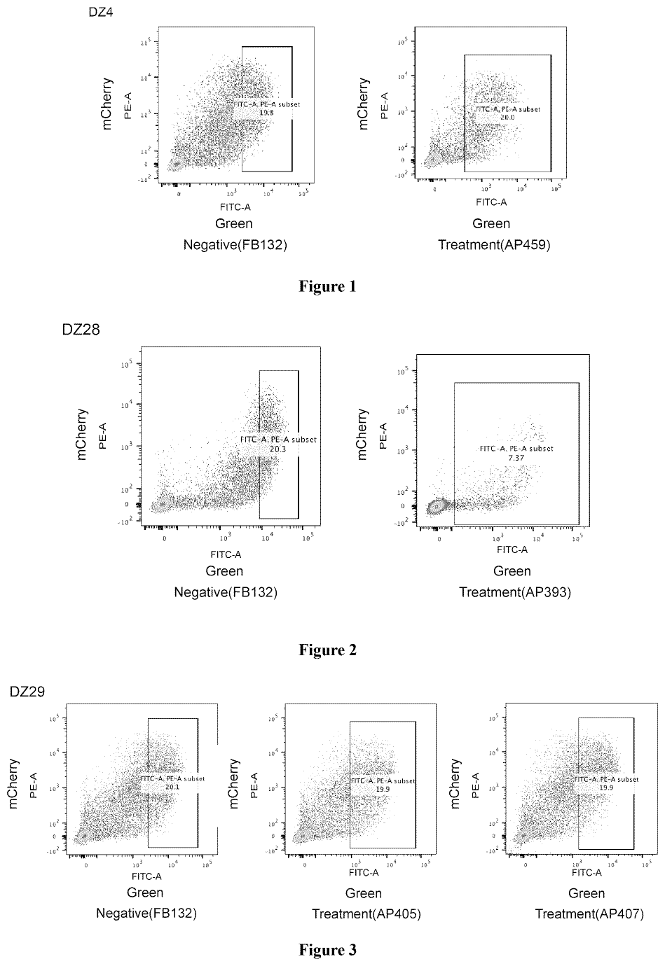

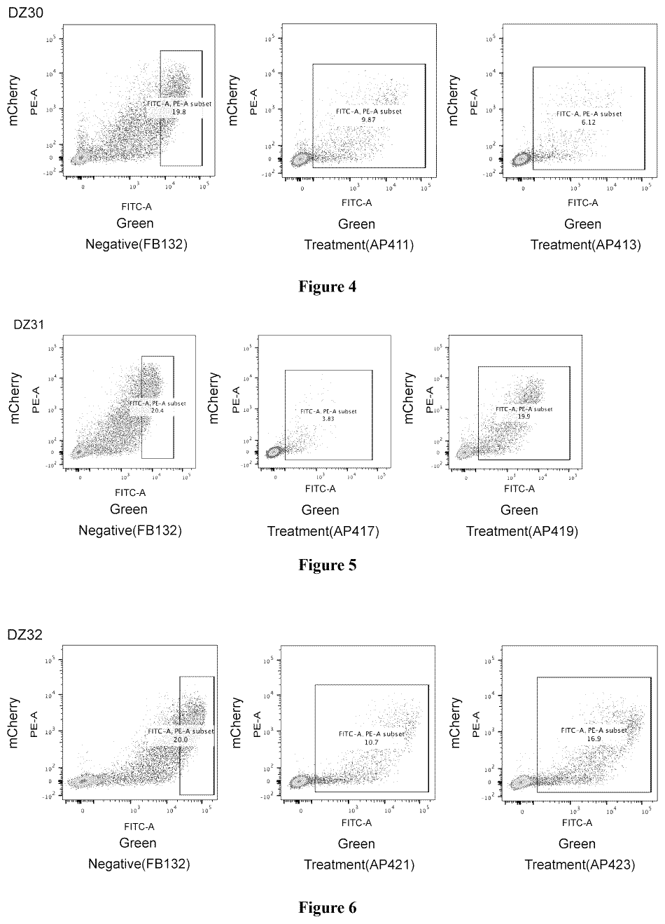

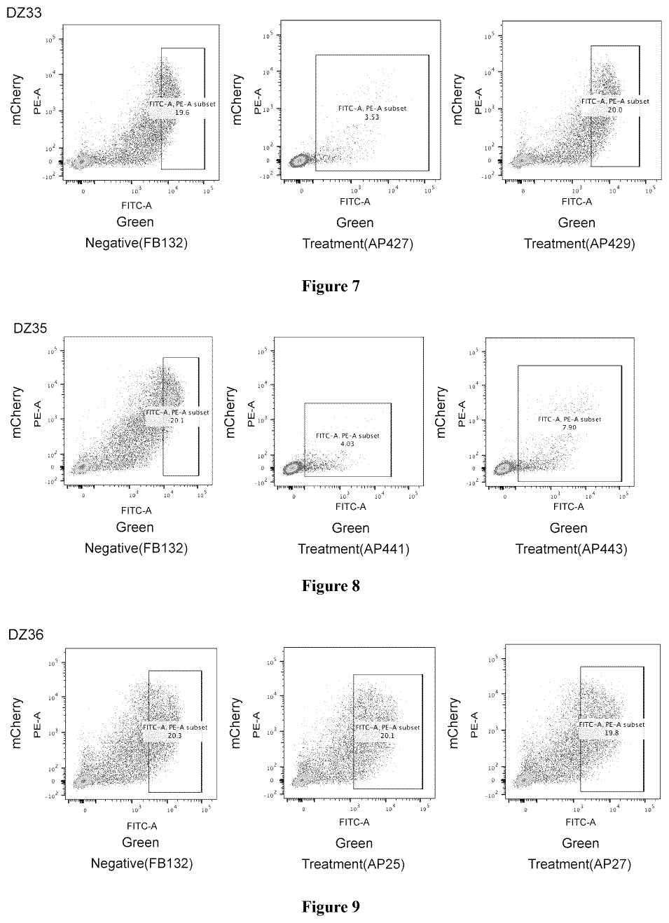

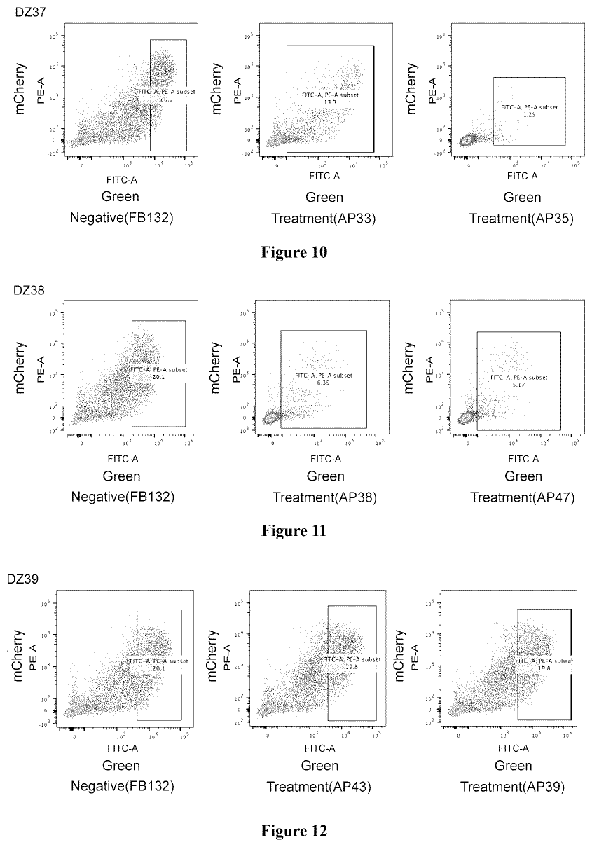

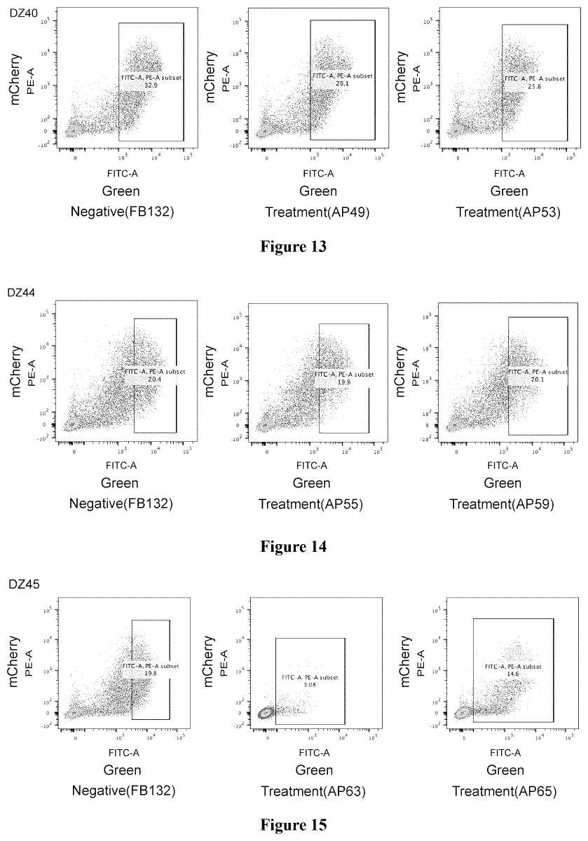

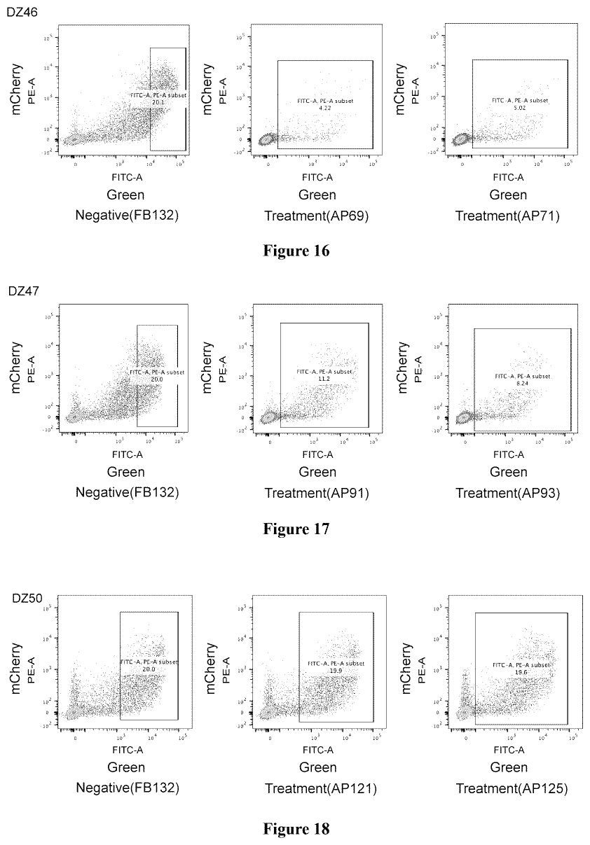

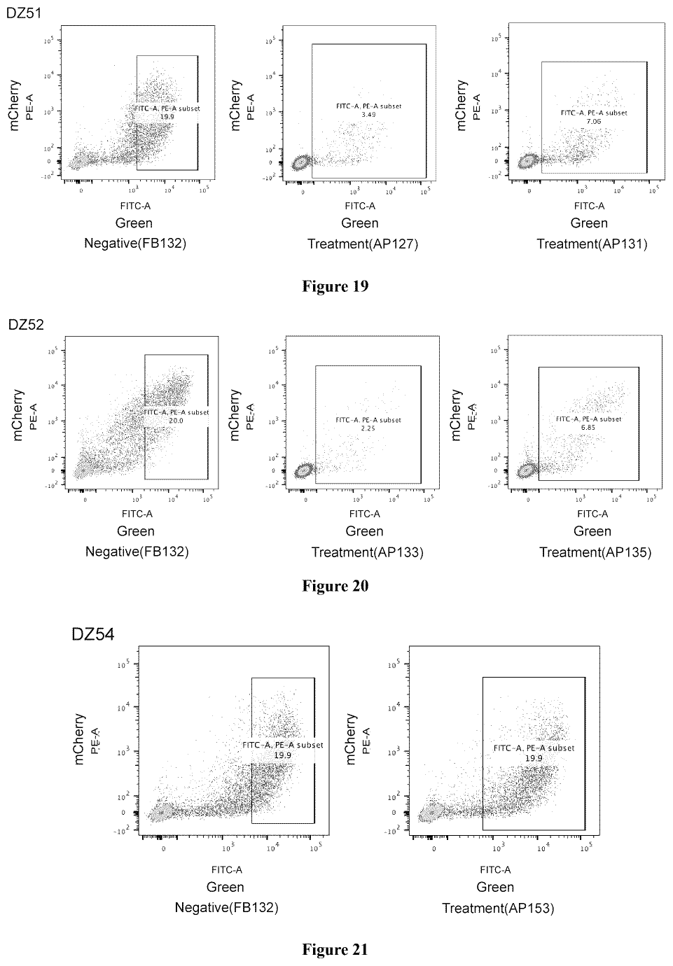

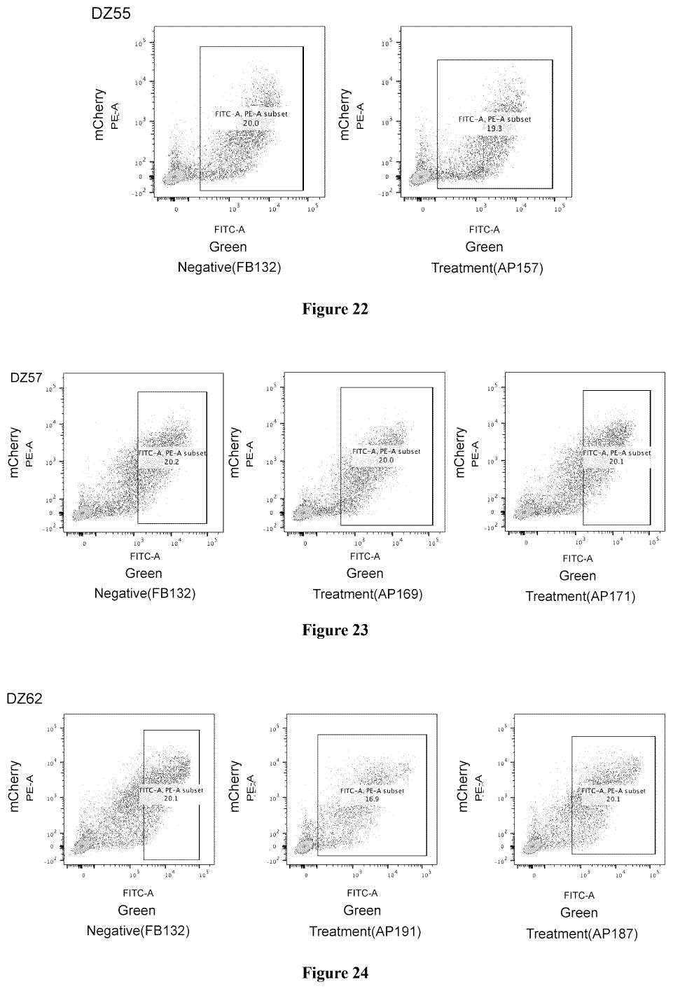

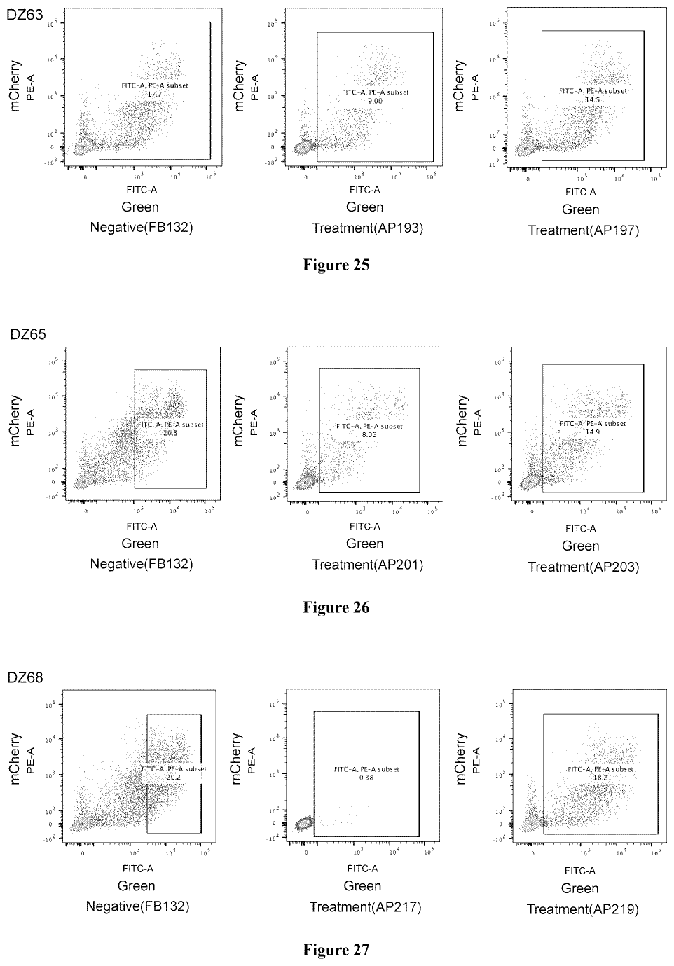

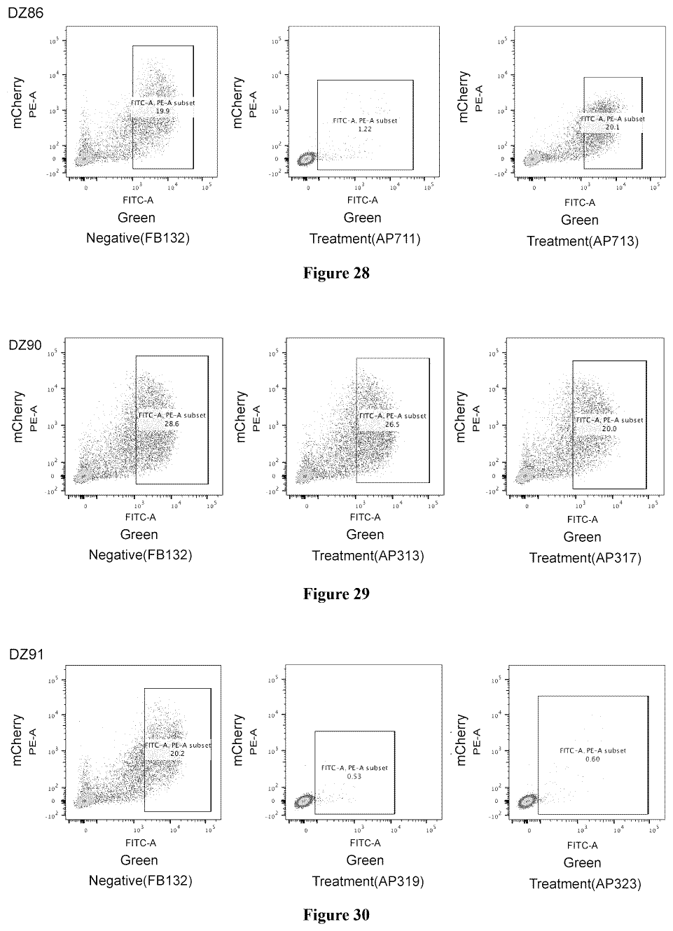

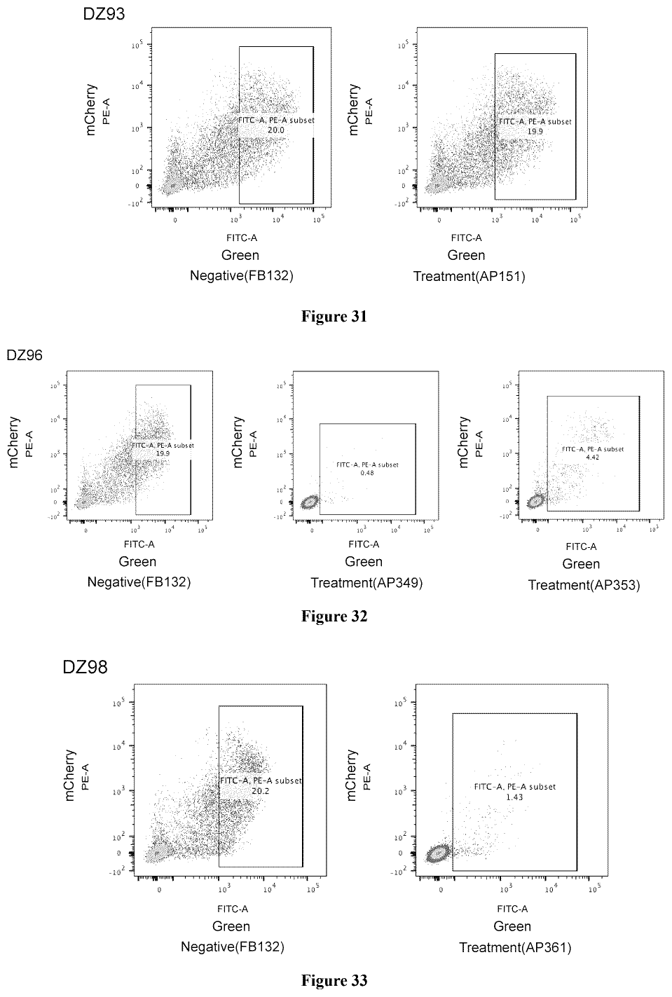

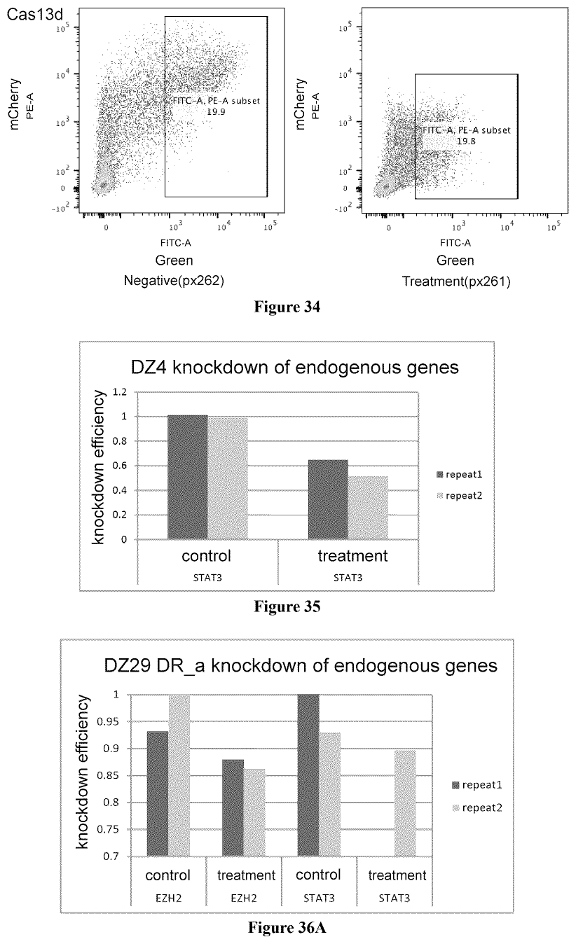

The results are shown in FIGS. 1 to 33, the results of DZ4, DZ28, DZ29, DZ30, DZ31, DZ32, DZ33, DZ35, DZ36, DZ37, DZ38, DZ39, DZ40, DZ44, DZ45, DZ46, DZ47, DZ50, DZ51, DZ52, DZ54, DZ55, DZ57, DZ62, DZ63, DZ65, DZ68, DZ86, DZ90, DZ91, DZ93, DZ96, DZ98 and the positive control protein Cas13d show that the Cas13 proteins we screened can perform effectively knocking down of the mCherry (red light) protein after transiently transfected mammalian cell lines, while the negative control group failed to be cleaved, exhibiting as red fluorescence highlights. These indicate that our candidate proteins have RNase activity in mammals and the like.

### Example 3: Functional Verification of Knocking Down 293T Endogenous Gene

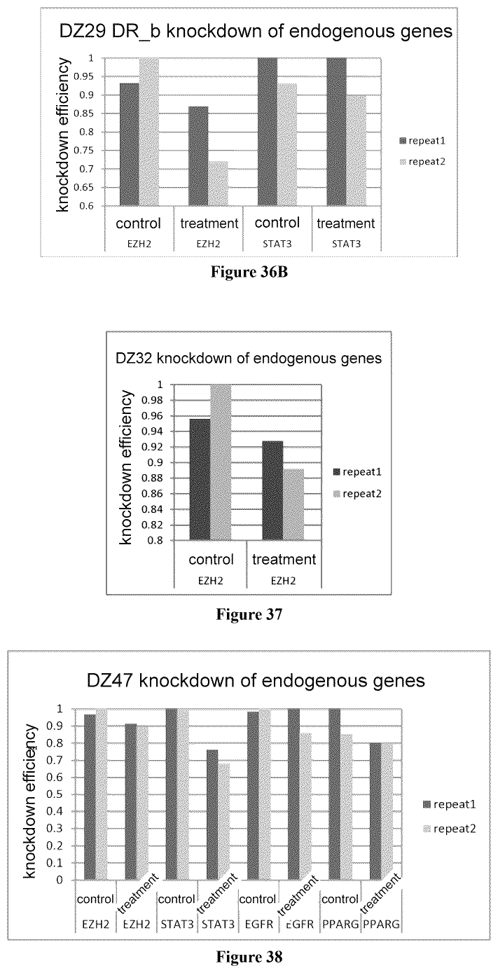

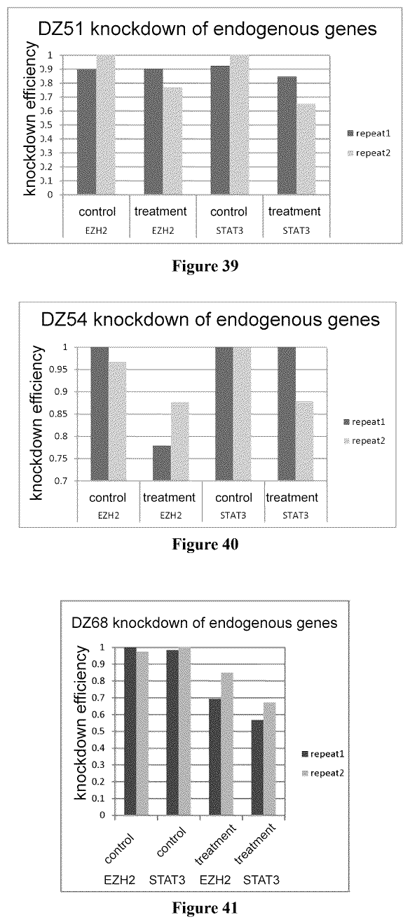

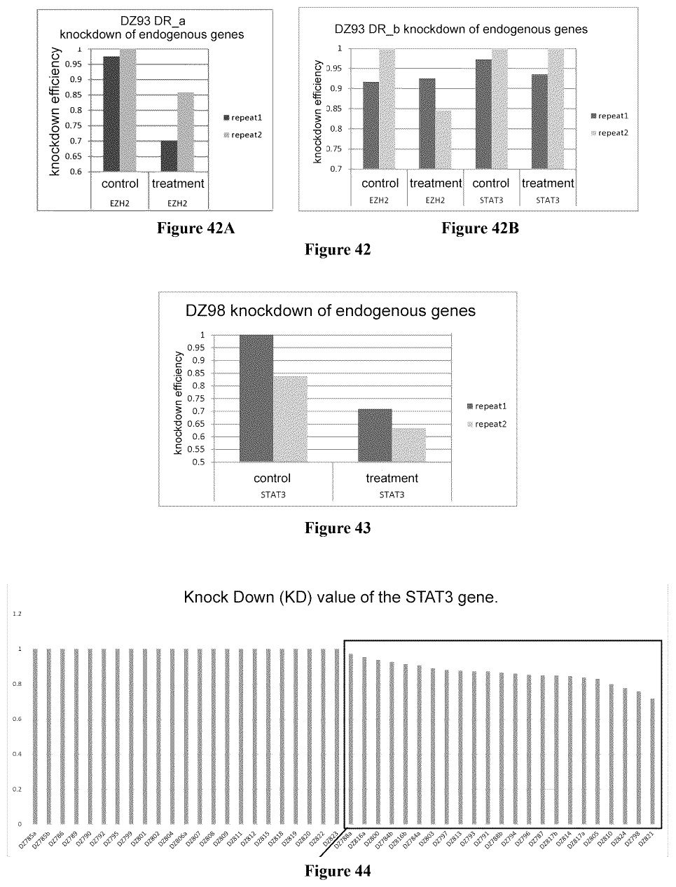

In order to further verify the ability of the candidate protein to cleave endogenous genes, we further screened some proteins from example 2 with high RNase activity against mCherry, including DZ4, DZ29, DZ32, DZ47, DZ51, DZ54, DZ68, DZ93, DZ98 and the like, to validate the knocking down efficiency of endogenous genes (STAT3, EZH2). We randomly designed sgRNA for the two endogenous genes of 293T. The cleavage results are shown in FIG. 35-43. The results show that all proteins have cleavage function. Although there is no significant difference between some of the experimental groups and the control group, this may be related to the PFS of protein. Previous studies have reported Cas13 proteins, such as Cas13a, Cas13b, exhibit strong PFS when targeting RNA sequences. The results from qPCR fully demonstrate the feasibility of our method for screening the candidate CRISPR-Cas proteins with Rase activity that are guided by guide RNAs.

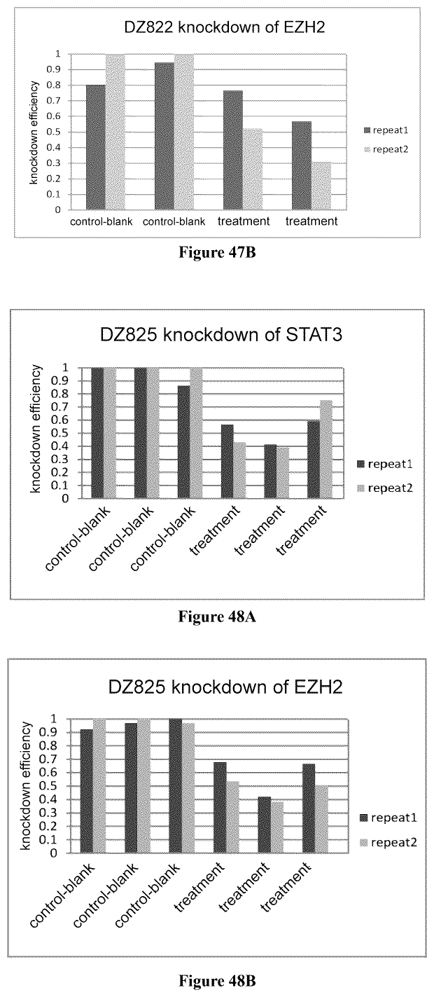

We directly conducted knockdown experiments on endogenous genes STAT3 and EZH2 using a subset of screened CRISPR-Cas proteins with RNase activity guided by guide RNAs (including dz806, dz825, dz822, dz821, etc.). The results are shown in FIGS. 44 to 48.

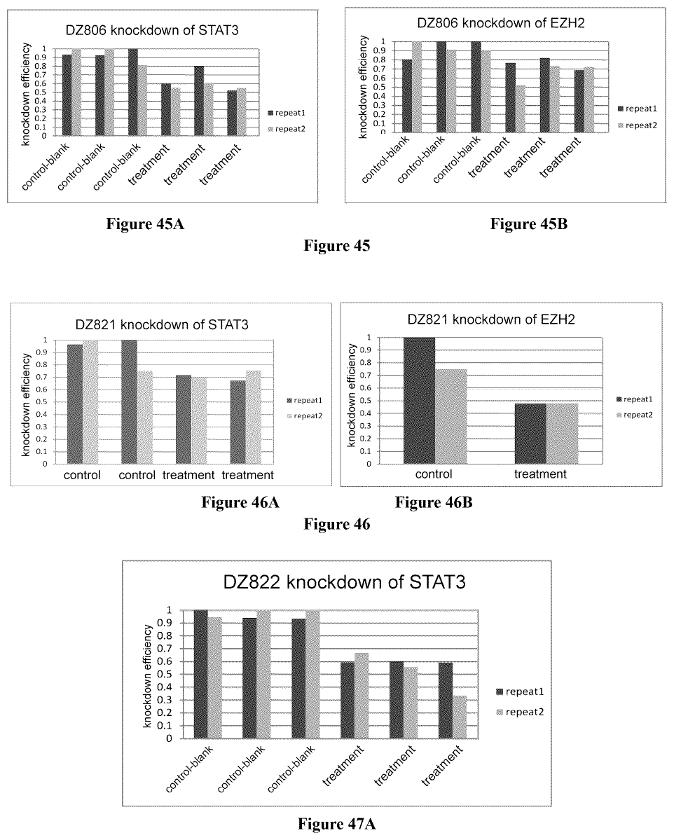

It can be found that although the PFS of the protein is unknown, there are still some candidate proteins that show a certain effect in knocking down the endogenous gene STAT3, including DZ784, DZ787, DZ788, DZ791, DZ793, DZ794, DZ796, DZ797, DZ798, DZ800, DZ803, DZ805, DZ810, DZ813, DZ814, DZ816, DZ817, DZ821 and DZ824. Subsequently, we further conducted knockdown experiments on the endogenous gene EZH2 using DZ806, DZ810, DZ821, DZ822, and DZ825. The results are shown in FIGS. 45 to 48. Repeated experiments consistently demonstrated that they have a certain knockdown effect on the endogenous genes EZH2 and STAT3.

### Example 4: PFS Function Screening of Candidate Cas13 Proteins

The screened candidate proteins can be further screened for their PFS through techniques known to those skilled in the art, which may improve the cleavage efficiency of the screened enzyme, etc.

In order to further explore the PFS of candidate proteins for targeting RNA, we designed detection experiments to find protein PFS. The detection method is: First, a library plasmid with a 5′-6N (NNNNNN)-spacer (target sequence)-antibiotic resistance gene or a spacer (target sequence)-NNNNNN (6N)-3′-antibiotic resistance gene was constructed (collectively referred to as the 6N library plasmid). Simultaneously, a guide RNA plasmid targeting the sequence of interest was designed. The 6N library plasmids were transfected into E. coli, along with the candidate protein plasmids and the corresponding guide RNA targeting the region of interest. A negative control was established by co-transfecting the candidate protein-related plasmids with a guide RNA that does not target the region of interest (i.e., nonTarget). Subsequently, all surviving E. coli were extracted and subjected to deep-sequencing. Bioinformatic methods were then employed to analyze the differential 5′ or 3′ preference sequences between the experimental and control groups, thereby calculating the PFS of the corresponding protein.

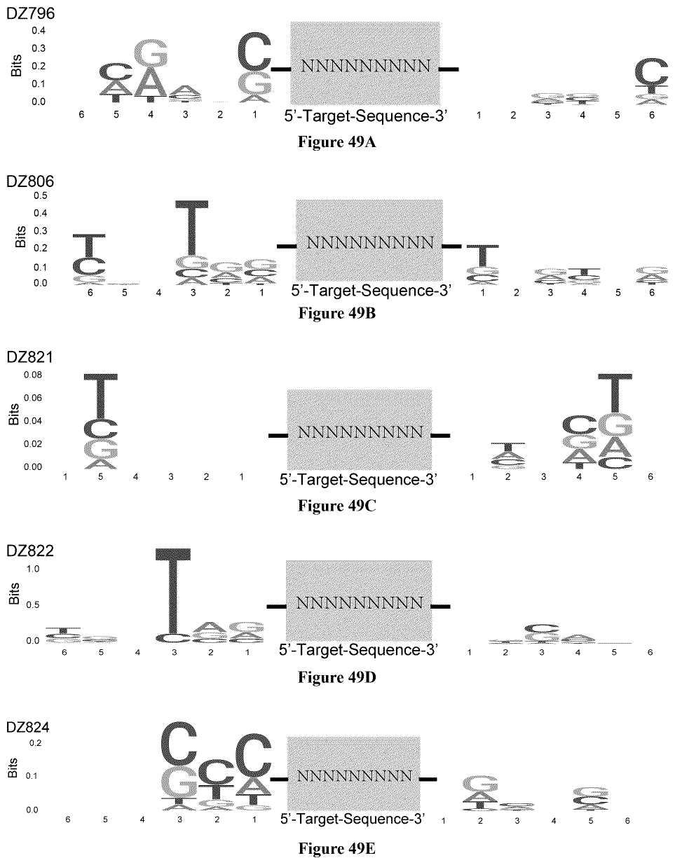

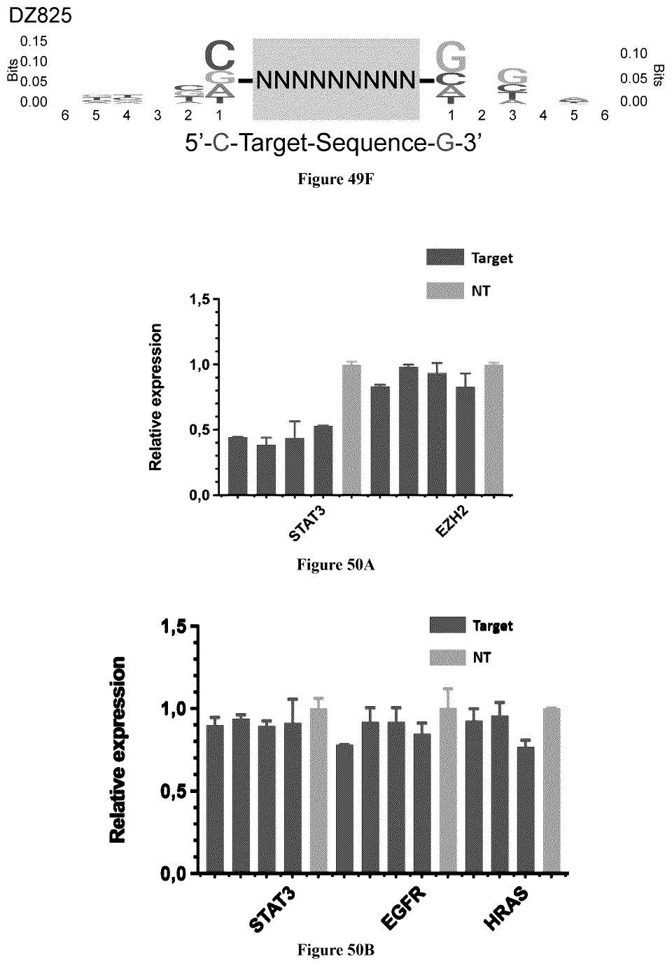

According to this method, we tested proteins numbered DZ796, DZ806, DZ821, DZ822, DZ824, and DZ825. As shown in FIG. 49, their potential PFS are 5′-[C/G]-targetSeq-NNNNN[G]. -3′ (FIG. 49A), 5′-T[G/A][G/A]-targetSeq-TN[G/A]-3′ (FIG. 49B), 5′-TNNN[C/G]-targetSeq-NNNNT-3′ (FIG. 49C), 5′-TN[G/C/A]-targetSeq-[G/A][C/G][G/C]-3′ (FIG. 49D), 5′-N[C/G][C/G][C/T]-targetSeq-NG[C/A]-3′ (FIG. 49E) and 5′-C-target-G-3′ (FIG. 49F).

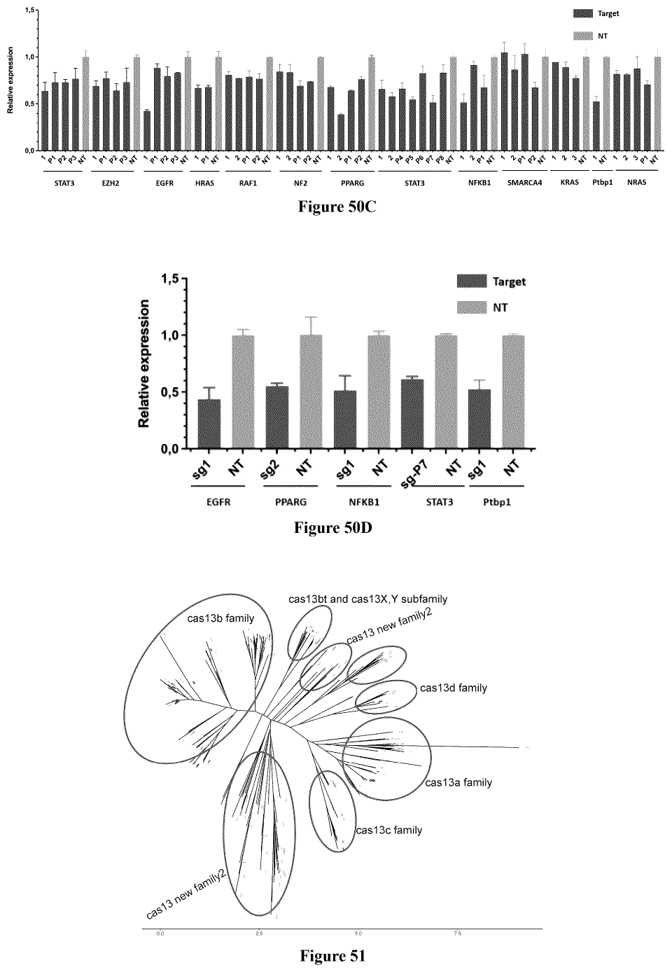

We then designed knock down experiments based on the PFS screened for DZ825, DZ822, and DZ806 proteins to knock down endogenous genes in a mammalian cell line (293T), as shown in FIG. 50. FIG. 50A shows the experimental results of using DZ825 protein to knock down (KD) the endogenous genes STAT3 and EZH2 in 293T cell lines. The first one in each gene experimental group uses a randomly designed spacer without prior knowledge of PFS, while the subsequent three groups use newly designed spacers based on the PFS motif. As observed in the figure, in the KD experiment of the 293T cell lines, some of the newly designed sgRNAs can still exhibit better KD effects. FIG. 50B shows the experimental results of using DZ822 protein to knock down (KD) the three endogenous genes STAT3, EGFR and HRAS in 293T cell lines. The first one in each gene experimental group uses a designed spacer without prior knowledge of PFS, while the subsequent groups use newly designed spacers based on the PFS motif. As observed in the figure, in the KD experiment of the 293T cell lines, some of the newly designed sgRNA can still exhibit better KD effects, such as KRAS. FIG. 50C shows the experimental results of using DZ806 protein based on its PFS design to knock down (KD) the endogenous genes in 293T cells. The selected endogenous genes include STAT3, EZH2, EGFR, HRAS, RAF1, NF2, SMARCA4, NFKB1, PPARG, KRAS, PTBP1, and NRAS. The first one in each KD gene experimental group use a designed spacer without prior knowledge of PFS, while the subsequent groups use newly designed spacers based on the PFS motif. As observed in the figure, in the KD experiment of the 293T cell lines, some of the newly designed sgRNAs can exhibit better KD effects, such as NF2 and SMARCA4.

### Example 5: Verification of Base Editing Function

Through mutating the cleavage domain (extended HEPN domain) of the candidate Cas13 proteins, candidate dCas13 proteins that only bind to RNA without cleavage activity is obtained. Then these are fused with adar enzyme sequence to construct plasmids for the ABE single base editing system. Next, we design the sgRNAs for targeted base mutation treatment of specific sequences, such as the transcript of the TP53 gene, construct the corresponding plasmid vector, and co-transfect into human 293T cell lines. Flow cytometry was performed after 48 hours to obtain the co-transfected cell lines. Then extract the RNA transcripts and build the library. Perform deep seq sequencing. After sequencing, the mutation status of TP53 gene transcripts is analyzed through bioinformatics methods to obtain the corresponding single-based editing efficiency of the ABE system. This allows for continuous optimization of sgRNA to achieve the construction of an optimal single-base editing system for the target region.

### Example 6: Homology Analysis of Candidate Cas13 Proteins and the Known Cas13 Proteins

This is based on the principle that the higher the coverage and the greater similarity of the unknown protein compared to the known protein, and thus the closer the homology between the unknown protein and the known protein. After screening the candidate proteins, we first downloaded the related protein sequences of Cas13a, b, c, d, x(e), y(f), and bt from the NCBI database and patent documents, then merge them with our data to construct a local blastp index file. Subsequently, we perform protein sequence alignment analysis between the candidate protein sequences and the sequences in the local blastp index database. For protein sequences with a similarity (identity) of less than 20% or those that cannot be aligned to the local database, we uniformly label them as 20%. Similarly, for those with a coverage of less than 5% or that cannot be aligned to the local database, we mark them as 1%. Most of the new Cas13 proteins identified by the method of the present invention have extremely low homology levels with the known Cas13 proteins from various families. Among them, the proteins DZ28, DZ29, DZ30, DZ31, DZ32, DZ33, DZ35, DZ36, DZ37, DZ40, DZ44, DZ45, DZ46, DZ47, DZ50, DZ51, DZ52, DZ54, DZ55, DZ57, DZ63, DZ65, DZ68, DZ86, DZ91, DZ98, DZ784, DZ785, DZ786, DZ787, DZ788, DZ789, DZ793, DZ795, DZ797, DZ798, DZ799, DZ801, DZ803, DZ804, DZ805, DZ806, DZ807, DZ809, DZ810, DZ812, DZ813, DZ81, DZ815, DZ816, DZ817, DZ819, DZ820, DZ821, DZ822, DZ825, DZ826, DZ827, DZ829, DZ831, DZ844 exhibit homology of less than 20% with the currently known Cas13 categories. The proteins DZ4, DZ38, DZ843, DZ62, DZ93, DZ794, DZ796, DZ824, DZ828 exhibit similarity from 20% to 50% with the known Cas13 protein family. The similarity of the remaining proteins is from 50% and 80% compared to the known proteins.

As shown in FIG. 51, further analysis of the evolutionary tree shows the candidate CRISPR-Cas13 proteins with RNase activity guided by the guide RNA that we screened have independent branches. Potentially, these belong to two relatively large and compact new Cas13 families, which are tentatively designated as the Cas13 ml family and the Cas13 m2 family. Among them, the Cas13 ml family such as DZ30, DZ32, etc.; the Cas13m2 family such as DZ47, DZ29, etc.

The DR sequence of the candidate Cas13 protein is shown in Table 1 below.

TABLE 1
DR sequences of candidate Cas13 proteins

| SEQ_ | | |
|---|---|---|
| ID_ | | |
| No. | DR-ID | DR-SEQ |
| 79 | DZ4a | tcttcaaattgtgatacgtcccaa |
| 80 | DZ4b | ttgggacgtatcacaatttgaaga |
| 81 | DZ28a | GTTTCCATTCAATTAATTGCCTCTATTAAAAGAGAC |
| 82 | DZ28b | GTCGTCCCCGCGCCCGCGGGGGTTGCTC |
| 83 | DZ29a | GTCGTCCCCGCGCCCGCGGGGGTTGCTCC |
| 84 | DZ29b | GGAGCAACCCCCGCGGGCGCGGGGACGAC |
| 85 | DZ30a | GTTTGCCATCGCCCAGATGGTTTAGAAG |
| 86 | DZ30b | CTTCTAAACCATCTGGGCGATGGCAAAC |
| 87 | DZ31a | GTCCTCATCGCCCCTACGAGGGGTCGCAAC |
| 88 | DZ31b | GTTGCGACCCCTCGTAGGGGCGATGAGGAC |
| 89 | DZ32a | GTTCACTGCCGCGTAGGCAGCTCAGAAA |
| 90 | DZ32b | TTTCTGAGCTGCCTACGCGGCAGTGAAC |
| 91 | DZ33a | GTGGCGGTCGCCCCTCGGGGGGACCGAGGATCGCAAC |
| 92 | DZ33b | GTTGCGATCCTCGGTCCCCCCGAGGGGCGACCGCCAC |
| 93 | DZ35a | GTTCTCTCCGCGCGAGCGGAGGTGGTCCG |
| 94 | DZ35b | CGGACCACCTCCGCTCGCGCGGAGAGAAC |
| 95 | DZ36a | gttgtaattgctcttattttgaagggtatacacaac |
| 96 | DZ36b | gttgtgtatacccttcaaaataagagcaattacaac |
| 97 | DZ37a | gctgtactcacccttcaaataaagggcttttacagc |
| 98 | DZ37b | gctgtaaaagccctttatttgaagggtgagtacagc |
| 99 | DZ38a | gttgggaatacccttagttagaagggtggagacaac |
| 100 | DZ38b | GTTGGGAATACCCTTAGTTAGAAGGGTGGAGACAACT |
| 101 | DZ39a | gttgggaatacccttagttagaagggtggagacaac |
| 102 | DZ39b | gttgtctccacccttctaactaagggtattcccaac |
| 103 | DZ40a | gttgtagttccctgatcgttcttggtatggtataat |
| 104 | DZ40b | ATTATACCATACCAAGAACGATCAGGGAACTACAAC |
| 105 | DZ44a | aggatagcagttcagaaatcgcggtccagctgcaac |
| 106 | DZ44b | gttgcagctggaccgcgatttctgaactgctatcct |
| 107 | DZ45a | gggctcatccccgcacgcgcggggagcac |
| 108 | DZ45b | gtgctccccgcgcgtgcggggatgagccc |
| 109 | DZ46a | agtcttccccacatgggtgggggtgtttcta |
| 110 | DZ46b | gtcttccccacatgggtgggggtgtttcta |
| 111 | DZ47a | gttgcaaaggctgtccctcggtagagggattgaaac |
| 112 | DZ47b | gttgcaaaggctgtccctcggtagagggattgaaaca |
| | | c |
| 113 | DZ50a | cggaccatccccacgcacgtggggagaac |
| 114 | DZ50b | gttctccccacgtgcgtggggatggtccg |
| 115 | DZ51a | gtccgctttcatctaggaagtggaattaatggaaac |
| 116 | DZ51b | gtttccattaattccacttcctagatgaaagcggac |
| 117 | DZ52a | gtcgcagctccttcgggagctgctcttcattgaggc |
| 118 | DZ52b | gcctcaatgaagagcagctcccgaaggagctgcgac |
| 119 | DZ54a | gtcgcgccccgcacggggcgcgtggattgaaac |
| 120 | DZ54b | gtttcaatccacgcgccccgtgcggggcgcg |
| 121 | DZ55a | ggtgtgaaagccatctttttgtatggtagggacacc |
| 122 | DZ55b | ggtgtccctaccatacaaaaagatggctttcacacc |
| 123 | DZ57a | gtcgctcccctcgcgggagcgtggattgaaata |
| 124 | DZ57b | tatttcaatccacgctcccgcgaggggagcgac |
| 125 | DZ62a | aaataccacccaagaatgagggggttctataacc |
| 126 | DZ62b | ggttatagaaccccctcattcttgggtggtattt |
| 127 | DZ63a | agtttatccgatgggagatcggggaggaaccgcaac |
| 128 | DZ63b | gttgcggttcctccccgatctcccatcggataaact |
| 129 | DZ65a | cttccaatttgcgcgtgggcgtgagttgggggcac |
| 130 | DZ65b | gtgcccccaactcacgcccacgcgcaaattggaag |
| 131 | DZ68a | gtcgcagtcctcactaaaattggacatgac |
| 132 | DZ68b | gtcatgtccaattttagtgaggactgcgac |
| 133 | DZ86a | GCTGTGATAGACCTCGATTTGTGGGGTAGTAACAGC |
| 134 | DZ86b | GCTGTTACTACCCCACAAATCGAGGTCTATCACAGC |
| 135 | DZ90a | TGAATACAGCTCGATATAGTGAGCAATAACT |
| 136 | DZ90b | AGTTATTGCTCACTATATCGAGCTGTATTCA |
| 137 | DZ91a | GTTTCACCAGCCGATTTTTTAAACGGTAACTGAAAC |
| 138 | DZ91b | GTTTCAGTTACCGTTTAAAAAATCGGCTGGTGAAAC |
| 139 | DZ93a | GTTGTAGAAGCCACTTGTTTGAAATGGCATGACAAC |
| 140 | DZ93b | GTTGTCATGCCATTTCAAACAAGTGGCTTCTACAAC |
| 141 | DZ96a | GTTGGAGATCACCCCCAAATCGAGGGGGACTGCACC |
| 142 | DZ96b | GGTGCAGTCCCCCTCGATTTGGGGGTGATCTCCAAC |
| 143 | DZ98a | GAATCGCCCGGCTTCCCAGCCGGGCGCGGATTGAAAC |
| 144 | DZ98b | GTTTCAATCCGCGCCCGGCTGGGAAGCCGGGCGATTC |
| 145 | dz784a | GTTCAATTTTGAGTACTATA |
| 146 | dz784b | TATAGTACTCAAAATTGAAC |
| 147 | dz785a | GAGCATACGCACAAAGTCCACAGT |
| 148 | dz785b | ACTGTGGACTTTGTGCGTATGCTC |
| 149 | dz786a | GTTTTAGAGCTGTGCTGTTTCGAATGGTTCCAAAAC |
| 150 | dz786b | GTTTTGGAACCATTCGAAACAGCACAGCTCTAAAAC |
| 151 | dz787a | GTTTTAGAGCTGTGCTGTTTCGAATGGTTCCAAAAC |
| 152 | dz787b | GTTTTGGAACCATTCGAAACAGCACAGCTCTAAAAC |
| 153 | dz788a | GGTTCACCCGCGCACGCGCGTGTAAGG |
| 154 | dz788b | CCTTACACGCGCGTGCGCGGGTGAACC |
| 155 | dz789a | GTCTCCCTCCATGCGGAGGGAGTGGATTGAAAT |
| 156 | dz789b | ATTTCAATCCACTCCCTCCGCATGGAGGGAGAC |
| 157 | dz790a | GTTGTAGTTCCCTTTCATTTTGGGATCATTCACACC |
| 158 | dz790b | GGTGTGAATGATCCCAAAATGAAAGGGAACTACAAC |
| 159 | dz791a | GTTGTAGAAGCCTATCGTTTGGATAGGTATGACAAC |
| 160 | dz791b | GTTGTCATACCTATCCAAACGATAGGCTTCTACAAC |
| 161 | dz793a | GTTCGCTGCCGCGCAGGCAGCTCAGAAA |
| 162 | dz793b | TTTCTGAGCTGCCTGCGCGGCAGCGAAC |
| 163 | dz794a | GTTGCACCGACCACGCCCACTGAAGGGCGACTGCACC |
| 164 | dz794b | GGTGCAGTCGCCCTTCAGTGGGCGTGGTCGGTGCAAC |
| 165 | dz795a | GTCGCTCCCCATTCGGGGAGCGTGGATTGAAAT |
| 166 | dz795b | ATTTCAATCCACGCTCCCCGAATGGGGAGCGAC |
| 167 | dz796a | GTTGTAGAAGCCCTCATTTTGAGAGGGTATAACAAC |
| 168 | dz796b | GTTGTTATACCCTCTCAAAATGAGGGCTTCTACAAC |
| 169 | dz797a | GTTTTAGATATAAGTCATTTTAAGTACATAGAACCC |
| 170 | dz797b | GGGTTCTATGTACTTAAAATGACTTATATCTAAAAC |
| 171 | dz798a | GTGGCGACGGGTGAGGAGGCCGGATCGGGTTGGAGG |
| 172 | dz798b | CCTCCAACCCGATCCGGCCTCCTCACCCGTCGCCAC |
| 173 | dz799a | GTTTTTATCGTCCCTATAAGGGGTTGAAAC |
| 174 | dz799b | GTTTCAACCCCTTATAGGGACGATAAAAAC |
| 175 | dz800a | GTTGTAGTTCCCTTTCATTTTGGGATCATTCACACC |
| 176 | dz800b | GGTGTGAATGATCCCAAAATGAAAGGGAACTACAAC |
| 177 | dz801a | GCCCCCAACAAACCATCAGCCGAAAGGCGATTGAGAC |
| 178 | dz801b | GTCTCAATCGCCTTTCGGCTGATGGTTTGTTGGGGGC |
| 179 | dz802a | GTTGGAGATCACCCCCAAATCGAGGGGGACTGCACC |
| 180 | dz802b | GGTGCAGTCCCCCTCGATTTGGGGGTGATCTCCAAC |
| 181 | dz803a | GTCGAGGCTCGCGAGAGCCTTGTGGATTGAAAT |
| 182 | dz803b | ATTTCAATCCACAAGGCTCTCGCGAGCCTCGAC |
| 183 | dz804a | GTCGCCTTCCCCCCGGAAGGCGTGGATTGAAAC |
| 184 | dz804b | GTTTCAATCCACGCCTTCCGGGGGGAAGGCGAC |
| 185 | dz805a | GTTTGCCCCGCATGTGCGGGGATGATCCG |
| 186 | dz805b | CGGATCATCCCCGCACATGCGGGGCAAAC |
| 187 | dz806a | CTCCTTCTGCTCAGGCGTGGCTT |
| 188 | dz806b | AAGCCACGCCTGAGCAGAAGGAG |
| 189 | dz807a | CGTTTCCACGGCATCACAGCCGTGGCCGAATTGAAGC |
| 190 | dz807b | GCTTCAATTCGGCCACGGCTGTGATGCCGTGGAAACG |
| 191 | dz809a | GTAAGAATCAAATAATCCCGATACGCGGGATTAAGAC |
| 192 | dz809b | GTCTTAATCCCGCGTATCGGGATTATTTGATTCTTAC |
| 193 | dz810a | GCTGCATTCCCCGCGCGAGAGGGGATTGAGAC |
| 194 | dz810b | GTCTCAATCCCCTCTCGCGCGGGGAATGCAGC |
| 195 | dz811a | GTTGTGTGTACCCTTCGAATAGAGGGTAGATCCAAC |
| 196 | dz811b | GTTGGATCTACCCTCTATTCGAAGGGTACACACAAC |
| 197 | dz812a | GTCGCGCCTTCGCGGGCGCGTGAGTTGAAAC |
| 198 | dz812b | GTTTCAACTCACGCGCCCGCGAAGGCGCGAC |
| 199 | dz813a | GGTTCCCCCGTACACGCGGGGATAGACC |
| 200 | dz813b | GGTCTATCCCCGCGTGTACGGGGGAACC |
| 201 | dz814a | GTGCTCCCCGCACACGCGGGGATGATCCC |
| 202 | dz814b | GGGATCATCCCCGCGTGTGCGGGGAGCAC |
| 203 | dz815a | GGTGGAGACACGCGGATTTAGGGGTGTGATGACAGG |
| 204 | dz815b | CCTGTCATCACACCCCTAAATCCGCGTGTCTCCACC |
| 205 | dz816a | ATTCCTAAGCTTTTACGCTTAGGACTTCATTGAGG |
| 206 | dz816b | CCTCAATGAAGTCCTAAGCGTAAAAGCTTAGGAAT |
| 207 | dz817a | CCCTCAACTATTGAAACGTGTTTCAGTCGTTTCAGG |
| 208 | dz817b | CCTGAAACGACTGAAACACGTTTCAATAGTTGAGGG |
| 209 | dz819a | GGTTTCCGTCCCCGTGAAGGGGAAGTTGTATGAAAC |
| 210 | dz819b | GTTTCATACAACTTCCCCTTCACGGGGACGGAAACC |
| 211 | dz820a | TTATGTGCTCAGGGCCACTGCATGGTGCTGATGGAG |
| | | GCCAC |
| 212 | dz820b | GTGGCCTCCATCAGCACCATGCAGTGGCCCTGAGCA |
| | | CATAA |
| 213 | dz821a | GGTGTCGGAAACCGCTAATTCAGGGGCCGCTACAAC |
| 214 | dz821b | GTTGTAGCGGCCCCTGAATTAGCGGTTTCCGACACC |
| 215 | dz822a | AGTTTAGCAGATTGGGATTTGTACTCTGACCGGAAC |
| 216 | dz822b | GTTCCGGTCAGAGTACAAATCCCAATCTGCTAAACT |
| 217 | dz824a | GTAGAAATGAGTACAAAGCGATAGAGAGCTTAATAAC |
| 218 | dz824b | GTTATTAAGCTCTCTATCGCTTTGTACTCATTTCTAC |
| 219 | dz825a | AACTCGGAAGGATTCAGAAGAAGCTTTCATCT |
| 220 | dz825b | AGATGAAAGCTTCTTCTGAATCCTTCCGAGTT |
| 221 | dz826a | GTTCACTGCCGCACAGGCAGCTCAGAAA |
| 222 | dz826b | TTTCTGAGCTGCCTGTGCGGCAGTGAAC |
| 223 | dz827a | GTCTCCCTCCATGCGGAGGGAGTGGATTGAAAT |
| 224 | dz827b | ATTTCAATCCACTCCCTCCGCATGGAGGGAGAC |
| 225 | dz828a | GTTGAAAGAGAATAGCCCGACATAGTGGGCAATCAA |
| 226 | dz828b | TTGATTGCCCACTATGTCGGGCTATTCTCTTTCAAC |
| 227 | dz829a | GTTGTTCTCACCTTCCAAAATTAAGGCAT |
| 228 | dz829b | ATGCCTTAATTTTGGAAGGTGAGAACAAC |
| 229 | dz831a | CCTTCCGTGGCTGCAAAGCCACGGCCCCATTGAAGC |
| 230 | dz831b | GCTTCAATGGGGCCGTGGCTTTGCAGCCACGGAAGG |
| 231 | dz843a | GGTGTGGATGCCTCTATTTTGAGAGGTAGAATCACC |
| 232 | dz843b | GGTGATTCTACCTCTCAAAATAGAGGCATCCACACC |
| 233 | dz844a | GTCGCAGCTACAAGGCCGCCGCAATGGCCATTGGAAC |
| | | AT |
| 234 | dz844b | ATGTTCCAATGGCCATTGCGGCGGCCTTGTAGCTGCG |
| | | AC |

TABLE 2
Summary of sequence numbers and characteristics of cas13 candidate proteins

| | | Numbers | | |
|---|---|---|---|---|
| | | of | | |
| | | extended | Location of | |
| Seq | | HEPN | extended HEPN | |
| ID No. | Code | domains | domains | Extended HEPN domains |
| 1 | DZ4 | 2 | 312 451 | RFLLDH RNQFAH |
| 2 | DZ28 | 3 | 26 128 408 | RVIRKDCH RYFQQH RNDLFH |
| 3 | DZ29 | 6 | 26 161 163 188 367 369 | RRWYVH RARQELFH RQELFH RANAIASH RDRRPLPH |
| | | | | RRPLPH |
| 4 | DZ30 | 4 | 54 136 323 385 | RDNLGHFH RLFQTLIH RQKIPH RISVDWVH |
| 5 | DZ31 | 9 | 31 61 72 96 109 324 342 | RGVATH RVAEWMH RPYEGSQH RGVATVTH RHRGPH |
| | | | 364 366 | RTCSRGPH RDQLRH RGRAGPSH RAGPSH |
| 6 | DZ32 | 5 | 58 158 201 255 318 | RNRSRH RNIRLH RPLEQH RQLRNLRH RNLWGNH |
| 7 | DZ33 | 4 | 96 214 317 321 | RGYERH RAAATTDH RQGSRRH RRHRRH |
| 8 | DZ35 | 8 | 2 24 26 28 64 246 278 286 | RTGRPH RRRGRH RGRHRAGH RHRAGH RYRPDH |
| | | | | RIPEGSGH RINTQH RALRRH |
| 9 | DZ36 | 4 | 283 285 386 388 | RKRKDH RKDHSMLH RVRNCFSH RNCFSH |
| 10 | DZ37 | 5 | 70 253 451 470 482 | RDIAYWQH RRYARNEH RDYLKH RSDEEFEH RNRFAH |
| 11 | DZ38 | 7 | 208 302 356 514 561 573 606 | RAIVAELH RNKQAH RRELNIH RVPGLMSH RDLKPYLH |
| | | | | REGKSGEH RNKAAH |
| 12 | DZ39 | 4 | 69 78 123 372 | RWTKVYGH RRYLPFLH RNDFSH RTITDH |
| 13 | DZ40 | 7 | 70 193 356 415 417 461 543 | RTEFEH RCAADH RAGLLH RHRQLLCH RQLLCH |
| | | | | RPDQGPHH RPLVSH |
| 14 | DZ44 | 4 | 6 63 222 378 | RIGAVLIH RYGESSH RYDLCH RNRIVH |
| 15 | DZ45 | 6 | 30 259 300 364 450 472 | RHLQAH RLDETH RLDDTSSH RSTIVH RAQWRSH |
| | | | | RAAAPVH |
| 16 | DZ46 | 2 | 51 156 | RMKVILH RLVINNNH |
| 17 | DZ47 | 3 | 84 131 165 | RIFRGAH RGTYRWH RVWNRIMH |
| 18 | DZ50 | 3 | 20 187 189 | RLFSDH RPRRSCSH RRSCSH |
| 19 | DZ51 | 2 | 132 166 | RHEWIKH RNLFIEH |
| 20 | DZ52 | 2 | 112 246 | RIDEHTH RISWVKGH |
| 21 | DZ54 | 4 | 55 139 202 224 | REIFVTH RVTFFDIH RPYEKHH RNLLLYH |
| 22 | DZ55 | 2 | 6 129 | RKAKPQQH RNYHSH |
| 23 | DZ57 | 1 | 476 | RVGNANH |
| 24 | DZ62 | 2 | 138 302 | RIIQNEH RNAIAH |
| 25 | DZ63 | 6 | 204 401 428 465 515 547 | RKAQELHH RNILPH RNAEAH RFPNPTVH RQQRSNEH |
| | | | | RLCNYKPH |
| 26 | DZ65 | 5 | 29 195 201 352 466 | RRCARH RQIPLH RTDGTH RSEIVH RCARCGH |
| 27 | DZ68 | 2 | 38 141 | RLWLSYQH RNQLAH |
| 28 | DZ86 | 3 | 77 557 763 | RNFYSH RGFVKEH RNAALH |
| 29 | DZ90 | 2 | 4 457 | RKELLINH RNGIDH |
| 30 | DZ91 | 3 | 9 62 264 | RDIGAH RQTKNH RRLEKNLH |
| 31 | DZ93 | 2 | 159 358 | RLKSLLAH RNAFGHNH |
| 32 | DZ96 | 4 | 154 337 473 559 | RRIHEH REGKVIH RHSAFH RVLLRTSH |
| 33 | DZ98 | 6 | 40 44 88 213 376 479 | RLGSRAIH RAIHIGQH RLGSRAIH RFGRAGH RLGSRAIH |
| | | | | RAIAEGH |
| 34 | dz784 | 2 | 59 94 | RIEDFTVH RLISIISH |
| 35 | dz785 | 2 | 26 116 | RDFHPAH RYCWQGSH |
| 36 | dz786 | 3 | 65 87 109 | RMVPKH RMVPKH RMVPKH |
| 37 | dz787 | 2 | 65 87 | RMVPKH RMVPKH |
| 38 | dz788 | 3 | 66 135 258 | RKVFTH REHCDHH RHHSERH |
| 39 | dz789 | 3 | 101 137 142 | RRPDISIH RQKTSRH RHPARESH |
| 40 | dz790 | 3 | 46 159 161 | REGKFNLH RNRVAHYH RVAHYH |
| 41 | dz791 | 2 | 32 104 | RISLTGKH RSLPNNRH |
| 42 | dz793 | 2 | 85 165 | RGSELH RGSERH |
| 43 | dz794 | 2 | 45 65 | RGKQAAH REKKPAH |
| 44 | dz795 | 2 | 69 110 | RARVFWH RHPDHH |
| 45 | dz796 | 3 | 26 142 148 | RNILYH RVLTSYRH RHYTAH |
| 46 | dz797 | 3 | 20 22 125 | RERKSQRH RKSQRH RLSAEYDH |
| 47 | dz798 | 3 | 185 192 216 | RGGLSGH RYILAH RSILHFH |
| 48 | dz799 | 2 | 137 139 | RDRYLYRH RYLYRH |
| 49 | dz800 | 2 | 212 223 | RNEMIKYH RTDELAH |
| 50 | dz801 | 3 | 77 159 173 | RRKADLVH RELNQNTH RDNCGH |
| 51 | dz802 | 2 | 219 231 | REIMRFGH RDIFEQNH |
| 52 | dz803 | 2 | 92 188 | RLIKWH RTILNNH |
| 53 | dz804 | 2 | 53 62 | RAVVSIH RGEGDLLH |
| 54 | dz805 | 7 | 16 75 77 138 199 216 259 | RIIPAH RLRIIPAH RIIPAH RIIPAH RIIPAH RDRTDH |
| | | | | RRIIPAH |
| 55 | dz806 | 2 | 2 140 | RSTGKHPH RAYSSH |
| 56 | dz807 | 2 | 116 126 | RRRITPH RIGLQFGH |
| 57 | dz809 | 2 | 12 76 | RDALEVFH RELEKVAH |
| 58 | dz810 | 2 | 99 123 | RLVRMH RDGLDEQH |
| 59 | dz811 | 2 | 78 146 | RSLILKH RNYYSH |
| 60 | dz812 | 4 | 6 33 35 83 | RKVSTH RAREGATH REGATH RRNRRRH |
| 61 | dz813 | 2 | 127 140 | RPDDTH RMAYLSRH |
| 62 | dz814 | 2 | 17 43 | RRLDSH RRLLPH |
| 63 | dz815 | 3 | 22 49 51 | RAAVLRPH RSRLFRAH RLFRAH |
| 64 | dz816 | 2 | 45 55 | RMAARH RDILEIH |
| 65 | dz817 | 2 | 9 24 | RASDFCH RNCIDAFH |
| 66 | dz819 | 2 | 3 35 | RLSAIAH RNHEMNH |
| 67 | dz820 | 3 | 27 53 67 | RTPCITRH RPALRALH RLPGDH |
| 68 | dz821 | 2 | 28 56 | RPKTCNH RNLSNH |
| 69 | dz822 | 3 | 20 23 64 | RNYRLH RLHWKPKH RNCMGQH |
| 70 | dz824 | 2 | 2 45 | RYYTKH RVVANIH |
| 71 | dz825 | 3 | 18 38 40 | RQCKGKAH RYRDPFIH RDPFIH |
| 72 | dz826 | 3 | 306 314 318 | RTGSSESH RIDARRH RRHAVVH |
| 73 | dz827 | 5 | 51 53 258 294 299 | RLRTSLDH RTSLDHQH RRPDISIH RQKTSRH RHPARESH |
| 74 | dz828 | 2 | 111 159 | RDYIDH RNYIITH |
| 75 | dz829 | 5 | 68 76 78 116 319 | RKPDELSH RNRLLVQH RLLVQH RNNASH RWIKSEH |
| 76 | dz831 | 2 | 201 206 | RKGAERLH RLHVGPH |
| 77 | dz843 | 3 | 129 195 246 | RNFQSH RFFDIH RRIFQH |
| 78 | dz844 | 4 | 37 51 227 266 | RVLAAH RYPHLH RLFERH RSAIWH |

The primers for plasmid construction of sgRNA for knocking down endogenous genes in the 293T cell line of the candidate Cas13 protein are shown in Table 3 below.

TABLE 3
sgRNA primers for targeted knockdown of endogenous genes

| Cas | | | | | Design | SEQ |
|---|---|---|---|---|---|---|
| protein | | | | | prin- | ID |
| ID | sgRNAID | primer | Sequence of primer | Notes | ciples | NO. |
| dz784a | ps1947 | F | cttgtggaaaggacgaaacaccgGTTCAATTATGAGTACTATAcaaat | targeting | random | 235 |
| | | | gctggtaacactgtggtccacaagg | EZH2 | design | |
| dz784a | ps1947 | R | acgcacactggacgcgcaaaaaaaTATAGTACTCATAATTGAACcctt | targeting | random | 236 |
| | | | gtggaccacagtgttaccagcatttg | EZH2 | design | |
| dz784a | ps1948 | F | cttgtggaaaggacgaaacaccgGTTCAATTATGAGTACTATAgtgca | targeting | random | 237 |
| | | | gctcctcagtcacaatcagggaagc | STAT3 | design | |
| dz784a | ps1948 | R | acgcacactggacgcgcaaaaaaaTATAGTACTCATAATTGAACgct | targeting | random | 238 |
| | | | tccctgattgtgactgaggagctgcac | STAT3 | design | |
| dz784b | ps1949 | F | cttgtggaaaggacgaaacaccgTATAGTACTCAAAATTGAACcaaa | targeting | random | 239 |
| | | | tgctggtaacactgtggtccacaagg | EZH2 | design | |
| dz784b | ps1949 | R | acgcacactggacgcgcaaaaaaaGTTCAATTTTGAGTACTATAcctt | targeting | random | 240 |
| | | | gtggaccacagtgttaccagcatttg | EZH2 | design | |
| dz784b | ps1950 | F | cttgtggaaaggacgaaacaccgTATAGTACTCAAAATTGAACgtgc | targeting | random | 241 |
| | | | agctcctcagtcacaatcagggaagc | STAT3 | design | |
| dz784b | ps1950 | R | acgcacactggacgcgcaaaaaaaGTTCAATTTTGAGTACTATAgctt | targeting | random | 242 |
| | | | ccctgattgtgactgaggagctgcac | STAT3 | design | |
| dz785a | ps1951 | F | cttgtggaaaggacgaaacaccgGAGCATACGCACAAAGTCCACA | targeting | random | 243 |
| | | | GTcaaatgctggtaacactgtggtccacaagg | EZH2 | design | |
| dz785a | ps1951 | R | acgcacactggacgcgcaaaaaaaACTGTGGACTTTGTGCGTATGC | targeting | random | 244 |
| | | | TCccttgtggaccacagtgttaccagcatttg | EZH2 | design | |
| dz785a | ps1952 | F | cttgtggaaaggacgaaacaccgGAGCATACGCACAAAGTCCACA | targeting | random | 245 |
| | | | GTgtgcagctcctcagtcacaatcagggaagc | STAT3 | design | |
| dz785a | ps1952 | R | acgcacactggacgcgcaaaaaaaACTGTGGACTTTGTGCGTATGC | targeting | random | 246 |
| | | | TCgcttccctgattgtgactgaggagctgcac | STAT3 | design | |
| dz785b | ps1953 | F | cttgtggaaaggacgaaacaccgACTGTGGACTTTGTGCGTATGCT | targeting | random | 247 |
| | | | Ccaaatgctggtaacactgtggtccacaagg | EZH2 | design | |
| dz785b | ps1953 | R | acgcacactggacgcgcaaaaaaaGAGCATACGCACAAAGTCCAC | targeting | random | 248 |
| | | | AGTccttgtggaccacagtgttaccagcatttg | EZH2 | design | |
| dz785b | ps1954 | F | cttgtggaaaggacgaaacaccgACTGTGGACTTTGTGCGTATGCT | targeting | random | 249 |
| | | | Cgtgcagctcctcagtcacaatcagggaagc | STAT3 | design | |
| dz785b | ps1954 | R | acgcacactggacgcgcaaaaaaaGAGCATACGCACAAAGTCCAC | targeting | random | 250 |
| | | | AGTgcttccctgattgtgactgaggagctgcac | STAT3 | design | |
| dz786 | ps1955 | F | cttgtggaaaggacgaaacaccgGTTTAAGAGCTGTGCTGTTTCGA | targeting | random | 251 |
| | | | ATGGTTCCTAAACcaaatgctggtaacactgtggtccacaagg | EZH2 | design | |
| dz786 | ps1955 | R | acgcacactggacgcgcaaaaaaaGTTTAGGAACCATTCGAAACA | targeting | random | 252 |
| | | | GCACAGCTCTTAAACccttgtggaccacagtgttaccagcatttg | EZH2 | design | |
| dz786 | ps1956 | F | cttgtggaaaggacgaaacaccgGTTTAAGAGCTGTGCTGTTTCGA | targeting | random | 253 |
| | | | ATGGTTCCTAAACgtgcagctcctcagtcacaatcagggaagc | STAT3 | design | |
| dz786 | ps1956 | R | acgcacactggacgcgcaaaaaaaGTTTAGGAACCATTCGAAACA | targeting | random | 254 |
| | | | GCACAGCTCTTAAACgcttccctgattgtgactgaggagctgcac | STAT3 | design | |
| dz787 | ps1955 | F | cttgtggaaaggacgaaacaccgGTTTAAGAGCTGTGCTGTTTCGA | targeting | random | 255 |
| | | | ATGGTTCCTAAACcaaatgctggtaacactgtggtccacaagg | EZH2 | design | |
| dz787 | ps1955 | R | acgcacactggacgcgcaaaaaaaGTTTAGGAACCATTCGAAACA | targeting | random | 256 |
| | | | GCACAGCTCTTAAACccttgtggaccacagtgttaccagcatttg | EZH2 | design | |
| dz787 | ps1956 | F | cttgtggaaaggacgaaacaccgGTTTAAGAGCTGTGCTGTTTCGA | targeting | random | 257 |
| | | | ATGGTTCCTAAACgtgcagctcctcagtcacaatcagggaagc | STAT3 | design | |
| dz787 | ps1956 | R | acgcacactggacgcgcaaaaaaaGTTTAGGAACCATTCGAAACA | targeting | random | 258 |
| | | | GCACAGCTCTTAAACgcttccctgattgtgactgaggagctgcac | STAT3 | design | |
| dz788a | ps1957 | F | cttgtggaaaggacgaaacaccgGGTTCACCCGCGCACGCGCGTG | targeting | random | 259 |
| | | | TAAGGcaaatgctggtaacactgtggtccacaagg | EZH2 | design | |
| dz788a | ps1957 | R | acgcacactggacgcgcaaaaaaaCCTTACACGCGCGTGCGCGGG | targeting | random | 260 |
| | | | TGAACCccttgtggaccacagtgttaccagcatttg | EZH2 | design | |
| dz788a | ps1958 | F | cttgtggaaaggacgaaacaccgGGTTCACCCGCGCACGCGCGTG | targeting | random | 261 |
| | | | TAAGGgtgcagctcctcagtcacaatcagggaagc | STAT3 | design | |
| dz788a | ps1958 | R | acgcacactggacgcgcaaaaaaaCCTTACACGCGCGTGCGCGGG | targeting | random | 262 |
| | | | TGAACCgcttccctgattgtgactgaggagctgcac | STAT3 | design | |
| dz788b | ps1959 | F | cttgtggaaaggacgaaacaccgCCTTACACGCGCGTGCGCGGGT | targeting | random | 263 |
| | | | GAACCcaaatgctggtaacactgtggtccacaagg | EZH2 | design | |
| dz788b | ps1959 | R | acgcacactggacgcgcaaaaaaaGGTTCACCCGCGCACGCGCGT | targeting | random | 264 |
| | | | GTAAGGccttgtggaccacagtgttaccagcatttg | EZH2 | design | |
| dz788b | ps1960 | F | cttgtggaaaggacgaaacaccgCCTTACACGCGCGTGCGCGGGT | targeting | random | 265 |
| | | | GAACCgtgcagctcctcagtcacaatcagggaagc | STAT3 | design | |
| dz788b | ps1960 | R | acgcacactggacgcgcaaaaaaaGGTTCACCCGCGCACGCGCGT | targeting | random | 266 |
| | | | GTAAGGgcttccctgattgtgactgaggagctgcac | STAT3 | design | |
| dz789 | ps1961 | F | cttgtggaaaggacgaaacaccgGTCTCCCTCCATGCGGAGGGAG | targeting | random | 267 |
| | | | TGGATTGAAATcaaatgctggtaacactgtggtccacaagg | EZH2 | design | |
| dz789 | ps1961 | R | acgcacactggacgcgcaaaaaaaATTTCAATCCACTCCCTCCGCA | targeting | random | 268 |
| | | | TGGAGGGAGACccttgtggaccacagtgttaccagcatttg | EZH2 | design | |
| dz789 | ps1962 | F | cttgtggaaaggacgaaacaccgGTCTCCCTCCATGCGGAGGGAG | targeting | random | 269 |
| | | | TGGATTGAAATgtgcagctcctcagtcacaatcagggaagc | STAT3 | design | |
| dz789 | ps1962 | R | acgcacactggacgcgcaaaaaaaATTTCAATCCACTCCCTCCGCA | targeting | random | 270 |
| | | | TGGAGGGAGACgcttccctgattgtgactgaggagctgcac | STAT3 | design | |
| dz790 | ps1963 | F | cttgtggaaaggacgaaacaccgGTTGTAGTTCCCTTTCATTTCGG | targeting | random | 271 |
| | | | GATCATTCACACCcaaatgctggtaacactgtggtccacaagg | EZH2 | design | |
| dz790 | ps1963 | R | acgcacactggacgcgcaaaaaaaGGTGTGAATGATCCCGAAATG | targeting | random | 272 |
| | | | AAAGGGAACTACAACccttgtggaccacagtgttaccagcatttg | EZH2 | design | |
| dz790 | ps1964 | F | cttgtggaaaggacgaaacaccgGTTGTAGTTCCCTTTCATTTCGG | targeting | random | 273 |
| | | | GATCATTCACACCgtgcagctcctcagtcacaatcagggaagc | STAT3 | design | |
| dz790 | ps1964 | R | acgcacactggacgcgcaaaaaaaGGTGTGAATGATCCCGAAATG | targeting | random | 274 |
| | | | AAAGGGAACTACAACgcttccctgattgtgactgaggagctgcac | STAT3 | design | |
| dz791 | ps1965 | F | cttgtggaaaggacgaaacaccgGTTGTAGAAGCCTATCGTTTGGA | targeting | random | 275 |
| | | | TAGGTATGACAACcaaatgctggtaacactgtggtccacaagg | EZH2 | design | |
| dz791 | ps1965 | R | acgcacactggacgcgcaaaaaaaGTTGTCATACCTATCCAAACGA | targeting | random | 276 |
| | | | TAGGCTTCTACAACccttgtggaccacagtgttaccagcatttg | EZH2 | design | |
| dz791 | ps1966 | F | cttgtggaaaggacgaaacaccgGTTGTAGAAGCCTATCGTTTGGA | targeting | random | 277 |
| | | | TAGGTATGACAACgtgcagctcctcagtcacaatcagggaagc | STAT3 | design | |
| dz791 | ps1966 | R | acgcacactggacgcgcaaaaaaaGTTGTCATACCTATCCAAACGA | targeting | random | 278 |
| | | | TAGGCTTCTACAACgcttccctgattgtgactgaggagctgcac | STAT3 | design | |
| dz793 | ps1969 | F | cttgtggaaaggacgaaacaccgGTTCGCTGCCGCGCAGGCAGCT | targeting | random | 279 |
| | | | CAGAAAcaaatgctggtaacactgtggtccacaagg | EZH2 | design | |
| dz793 | ps1969 | R | acgcacactggacgcgcaaaaaaaTTTCTGAGCTGCCTGCGCGGCA | targeting | random | 280 |
| | | | GCGAACccttgtggaccacagtgttaccagcatttg | EZH2 | design | |
| dz793 | ps1970 | F | cttgtggaaaggacgaaacaccgGTTCGCTGCCGCGCAGGCAGCT | targeting | random | 281 |
| | | | CAGAAAgtgcagctcctcagtcacaatcagggaagc | STAT3 | design | |
| dz793 | ps1970 | R | acgcacactggacgcgcaaaaaaaTTTCTGAGCTGCCTGCGCGGCA | targeting | random | 282 |
| | | | GCGAACgcttccctgattgtgactgaggagctgcac | STAT3 | design | |
| dz794 | ps1971 | F | cttgtggaaaggacgaaacaccgGTTGCACCGACCACGCCCACTG | targeting | random | 283 |
| | | | AAGGGCGACTGCACCcaaatgctggtaacactgtggtccacaagg | EZH2 | design | |
| dz794 | ps1971 | R | acgcacactggacgcgcaaaaaaaGGTGCAGTCGCCCTTCAGTGG | targeting | random | 284 |
| | | | GCGTGGTCGGTGCAACccttgtggaccacagtgttaccagcatttg | EZH2 | design | |
| dz794 | ps1972 | F | cttgtggaaaggacgaaacaccgGTTGCACCGACCACGCCCACTG | targeting | random | 285 |
| | | | AAGGGCGACTGCACCgtgcagctcctcagtcacaatcagggaagc | STAT3 | design | |
| dz794 | ps1972 | R | acgcacactggacgcgcaaaaaaaGGTGCAGTCGCCCTTCAGTGG | targeting | random | 286 |
| | | | GCGTGGTCGGTGCAACgcttccctgattgtgactgaggagctgcac | STAT3 | design | |
| dz795 | ps1973 | F | cttgtggaaaggacgaaacaccgGTCGCTCCCCATTCGGGGAGCGT | targeting | random | 287 |
| | | | GGATTGAAATcaaatgctggtaacactgtggtccacaagg | EZH2 | design | |
| dz795 | ps1973 | R | acgcacactggacgcgcaaaaaaaATTTCAATCCACGCTCCCCGAA | targeting | random | 288 |
| | | | TGGGGAGCGACccttgtggaccacagtgttaccagcatttg | EZH2 | design | |
| dz795 | ps1974 | F | cttgtggaaaggacgaaacaccgGTCGCTCCCCATTCGGGGAGCGT | targeting | random | 289 |
| | | | GGATTGAAATgtgcagctcctcagtcacaatcagggaagc | STAT3 | design | |
| dz795 | ps1974 | R | acgcacactggacgcgcaaaaaaaATTTCAATCCACGCTCCCCGAA | targeting | random | 290 |
| | | | TGGGGAGCGACgcttccctgattgtgactgaggagctgcac | STAT3 | design | |
| dz796 | ps1975 | F | cttgtggaaaggacgaaacaccgGTTGTAGAAGCCCTCAGTTTGAG | targeting | random | 291 |
| | | | AGGGTATAACAACcaaatgctggtaacactgtggtccacaagg | EZH2 | design | |
| dz796 | ps1975 | R | acgcacactggacgcgcaaaaaaaGTTGTTATACCCTCTCAAACTG | targeting | random | 292 |
| | | | AGGGCTTCTACAACccttgtggaccacagtgttaccagcatttg | EZH2 | design | |
| dz796 | ps1976 | F | cttgtggaaaggacgaaacaccgGTTGTAGAAGCCCTCAGTTTGAG | targeting | random | 293 |
| | | | AGGGTATAACAACgtgcagctcctcagtcacaatcagggaagc | STAT3 | design | |
| dz796 | ps1976 | R | acgcacactggacgcgcaaaaaaaGTTGTTATACCCTCTCAAACTG | targeting | random | 294 |
| | | | AGGGCTTCTACAACgcttccctgattgtgactgaggagctgcac | STAT3 | design | |
| dz797 | ps1977 | F | cttgtggaaaggacgaaacaccgGTTCTAGATATAAGTCAGTTTAA | targeting | random | 295 |
| | | | GTACATAGAACCCcaaatgctggtaacactgtggtccacaagg | EZH2 | design | |
| dz797 | ps1977 | R | acgcacactggacgcgcaaaaaaaGGGTTCTATGTACTTAAACTGA | targeting | random | 296 |
| | | | CTTATATCTAGAACccttgtggaccacagtgttaccagcatttg | EZH2 | design | |
| dz797 | ps1978 | F | cttgtggaaaggacgaaacaccgGTTCTAGATATAAGTCAGTTTAA | targeting | random | 297 |
| | | | GTACATAGAACCCgtgcagctcctcagtcacaatcagggaagc | STAT3 | design | |
| dz797 | ps1978 | R | acgcacactggacgcgcaaaaaaaGGGTTCTATGTACTTAAACTGA | targeting | random | 298 |
| | | | CTTATATCTAGAACgcttccctgattgtgactgaggagctgcac | STAT3 | design | |
| dz798 | ps1979 | F | cttgtggaaaggacgaaacaccgGTGGCGACGGGTGAGGAGGCCG | targeting | random | 299 |
| | | | GATCGGGTTGGAGGcaaatgctggtaacactgtggtccacaagg | EZH2 | design | |
| dz798 | ps1979 | R | acgcacactggacgcgcaaaaaaaCCTCCAACCCGATCCGGCCTCC | targeting | random | 300 |
| | | | TCACCCGTCGCCACccttgtggaccacagtgttaccagcatttg | EZH2 | design | |
| dz798 | ps1980 | F | cttgtggaaaggacgaaacaccgGTGGCGACGGGTGAGGAGGCCG | targeting | random | 301 |
| | | | GATCGGGTTGGAGGgtgcagctcctcagtcacaatcagggaagc | STAT3 | design | |
| dz798 | ps1980 | R | acgcacactggacgcgcaaaaaaaCCTCCAACCCGATCCGGCCTCC | targeting | random | 302 |
| | | | TCACCCGTCGCCACgcttccctgattgtgactgaggagctgcac | STAT3 | design | |
| dz799 | ps1981 | F | cttgtggaaaggacgaaacaccgGTTATTATCGTCCCTATAAGGGG | targeting | random | 303 |
| | | | TTGAAACcaaatgctggtaacactgtggtccacaagg | EZH2 | design | |
| dz799 | ps1981 | R | acgcacactggacgcgcaaaaaaaGTTTCAACCCCTTATAGGGACG | targeting | random | 304 |
| | | | ATAATAACccttgtggaccacagtgttaccagcatttg | EZH2 | design | |
| dz799 | ps1982 | F | cttgtggaaaggacgaaacaccgGTTATTATCGTCCCTATAAGGGG | targeting | random | 305 |
| | | | TTGAAACgtgcagctcctcagtcacaatcagggaagc | STAT3 | design | |
| dz799 | ps1982 | R | acgcacactggacgcgcaaaaaaaGTTTCAACCCCTTATAGGGACG | targeting | random | 306 |
| | | | ATAATAACgcttccctgattgtgactgaggagctgcac | STAT3 | design | |
| dz800 | ps1983 | F | cttgtggaaaggacgaaacaccgGTTGTAGTTCCCTTTCACTTTGG | targeting | random | 307 |
| | | | GATCATTCACACCcaaatgctggtaacactgtggtccacaagg | EZH2 | design | |
| dz800 | ps1983 | R | acgcacactggacgcgcaaaaaaaGGTGTGAATGATCCCAAAGTG | targeting | random | 308 |
| | | | AAAGGGAACTACAACccttgtggaccacagtgttaccagcatttg | EZH2 | design | |
| dz800 | ps1984 | F | cttgtggaaaggacgaaacaccgGTTGTAGTTCCCTTTCACTTTGG | targeting | random | 309 |
| | | | GATCATTCACACCgtgcagctcctcagtcacaatcagggaagc | STAT3 | design | |
| dz800 | ps1984 | R | acgcacactggacgcgcaaaaaaaGGTGTGAATGATCCCAAAGTG | targeting | random | 310 |
| | | | AAAGGGAACTACAACgcttccctgattgtgactgaggagctgcac | STAT3 | design | |
| dz801 | ps1985 | F | cttgtggaaaggacgaaacaccgGCCCCCAACAAACCATCAGCCG | targeting | random | 311 |
| | | | AAAGGCGATTGAGACcaaatgctggtaacactgtggtccacaagg | EZH2 | design | |
| dz801 | ps1985 | R | acgcacactggacgcgcaaaaaaaGTCTCAATCGCCTTTCGGCTGA | targeting | random | 312 |
| | | | TGGTTTGTTGGGGGCccttgtggaccacagtgttaccagcatttg | EZH2 | design | |
| dz801 | ps1986 | F | cttgtggaaaggacgaaacaccgGCCCCCAACAAACCATCAGCCG | targeting | random | 313 |
| | | | AAAGGCGATTGAGACgtgcagctcctcagtcacaatcagggaagc | STAT3 | design | |
| dz801 | ps1986 | R | acgcacactggacgcgcaaaaaaaGTCTCAATCGCCTTTCGGCTGA | targeting | random | 314 |
| | | | TGGTTTGTTGGGGGCgcttccctgattgtgactgaggagctgcac | STAT3 | design | |
| dz802 | ps1987 | F | cttgtggaaaggacgaaacaccgGTTGGAGATCACCCCCAAATCG | targeting | random | 315 |
| | | | AGGGGGACTGCACCcaaatgctggtaacactgtggtccacaagg | EZH2 | design | |
| dz802 | ps1987 | R | acgcacactggacgcgcaaaaaaaGGTGCAGTCCCCCTCGATTTGG | targeting | random | 316 |
| | | | GGGTGATCTCCAACccttgtggaccacagtgttaccagcatttg | EZH2 | design | |
| dz802 | ps1988 | F | cttgtggaaaggacgaaacaccgGTTGGAGATCACCCCCAAATCG | targeting | random | 317 |
| | | | AGGGGGACTGCACCgtgcagctcctcagtcacaatcagggaagc | STAT3 | design | |
| dz802 | ps1988 | R | acgcacactggacgcgcaaaaaaaGGTGCAGTCCCCCTCGATTTGG | targeting | random | 318 |
| | | | GGGTGATCTCCAACgcttccctgattgtgactgaggagctgcac | STAT3 | design | |
| dz803 | ps1989 | F | cttgtggaaaggacgaaacaccgGTCGAGGCTCGCGAGAGCCTTG | targeting | random | 319 |
| | | | TGGATTGAAATcaaatgctggtaacactgtggtccacaagg | EZH2 | design | |
| dz803 | ps1989 | R | acgcacactggacgcgcaaaaaaaATTTCAATCCACAAGGCTCTCG | targeting | random | 320 |
| | | | CGAGCCTCGACccttgtggaccacagtgttaccagcatttg | EZH2 | design | |
| dz803 | ps1990 | F | cttgtggaaaggacgaaacaccgGTCGAGGCTCGCGAGAGCCTTG | targeting | random | 321 |
| | | | TGGATTGAAATgtgcagctcctcagtcacaatcagggaagc | STAT3 | design | |
| dz803 | ps1990 | R | acgcacactggacgcgcaaaaaaaATTTCAATCCACAAGGCTCTCG | targeting | random | 322 |
| | | | CGAGCCTCGACgcttccctgattgtgactgaggagctgcac | STAT3 | design | |
| dz804 | ps1991 | F | cttgtggaaaggacgaaacaccgGTCGCCTTCCCCCCGGAAGGCGT | targeting | random | 323 |
| | | | GGATTGAAACcaaatgctggtaacactgtggtccacaagg | EZH2 | design | |
| dz804 | ps1991 | R | acgcacactggacgcgcaaaaaaaGTTTCAATCCACGCCTTCCGGG | targeting | random | 324 |
| | | | GGGAAGGCGACccttgtggaccacagtgttaccagcatttg | EZH2 | design | |
| dz804 | ps1992 | F | cttgtggaaaggacgaaacaccgGTCGCCTTCCCCCCGGAAGGCGT | targeting | random | 325 |
| | | | GGATTGAAACgtgcagctcctcagtcacaatcagggaagc | STAT3 | design | |
| dz804 | ps1992 | R | acgcacactggacgcgcaaaaaaaGTTTCAATCCACGCCTTCCGGG | targeting | random | 326 |
| | | | GGGAAGGCGACgcttccctgattgtgactgaggagctgcac | STAT3 | design | |
| dz805 | ps1993 | F | cttgtggaaaggacgaaacaccgGTTTGCCCCGCATGTGCGGGGAT | targeting | random | 327 |
| | | | GATCCGcaaatgctggtaacactgtggtccacaagg | EZH2 | design | |
| dz805 | ps1993 | R | acgcacactggacgcgcaaaaaaaCGGATCATCCCCGCACATGCG | targeting | random | 328 |
| | | | GGGCAAACccttgtggaccacagtgttaccagcatttg | EZH2 | design | |
| dz805 | ps1994 | F | cttgtggaaaggacgaaacaccgGTTTGCCCCGCATGTGCGGGGAT | targeting | random | 329 |
| | | | GATCCGgtgcagctcctcagtcacaatcagggaagc | STAT3 | design | |
| dz805 | ps1994 | R | acgcacactggacgcgcaaaaaaaCGGATCATCCCCGCACATGCG | targeting | random | 330 |
| | | | GGGCAAACgcttccctgattgtgactgaggagctgcac | STAT3 | design | |
| dz806a | ps1995 | F | cttgtggaaaggacgaaacaccgCTCCTTCTGCTCAGGCGTGGCTT | targeting | random | 331 |
| | | | caaatgctggtaacactgtggtccacaagg | EZH2 | design | |
| dz806a | ps1995 | R | acgcacactggacgcgcaaaaaaaAAGCCACGCCTGAGCAGAAGG | targeting | random | 332 |
| | | | AGccttgtggaccacagtgttaccagcatttg | EZH2 | design | |
| dz806a | ps1996 | F | cttgtggaaaggacgaaacaccgCTCCTTCTGCTCAGGCGTGGCTT | targeting | random | 333 |
| | | | gtgcagctcctcagtcacaatcagggaagc | STAT3 | design | |
| dz806a | ps1996 | R | acgcacactggacgcgcaaaaaaaAAGCCACGCCTGAGCAGAAGG | targeting | random | 334 |
| | | | AGgcttccctgattgtgactgaggagctgcac | STAT3 | design | |
| dz806b | ps1997 | F | cttgtggaaaggacgaaacaccgAAGCCACGCCTGAGCAGAAGGA | targeting | random | 335 |
| | | | Gcaaatgctggtaacactgtggtccacaagg | EZH2 | design | |
| dz806b | ps1997 | R | acgcacactggacgcgcaaaaaaaCTCCTTCTGCTCAGGCGTGGCT | targeting | random | 336 |
| | | | Tccttgtggaccacagtgttaccagcatttg | EZH2 | design | |
| dz806b | ps1998 | F | cttgtggaaaggacgaaacaccgAAGCCACGCCTGAGCAGAAGGA | targeting | random | 337 |
| | | | Ggtgcagctcctcagtcacaatcagggaagc | STAT3 | design | |
| dz806b | ps1998 | R | acgcacactggacgcgcaaaaaaaCTCCTTCTGCTCAGGCGTGGCT | targeting | random | 338 |
| | | | Tgcttccctgattgtgactgaggagctgcac | STAT3 | design | |
| dz806b | CP242 | F | cttgtggaaaggacgaaacaccgAAGCCACGCCTGAGCAGAAGGA | targeting | random | 339 |
| | | | Ggcggtgggctcggtcctgcgcttgcaggtc | SMARCA4 | design | |
| dz806b | CP242 | R | acgcacactggacgcgcaaaaaaaCTCCTTCTGCTCAGGCGTGGCT | targeting | random | 340 |
| | | | Tgacctgcaagcgcaggaccgagcccaccgc | SMARCA4 | design | |
| dz806b | CP243 | F | cttgtggaaaggacgaaacaccgAAGCCACGCCTGAGCAGAAGGA | targeting | random | 341 |
| | | | Gtccgagtccttcacccgtttgatctgctcc | HRAS | design | |
| dz806b | CP243 | R | acgcacactggacgcgcaaaaaaaCTCCTTCTGCTCAGGCGTGGCT | targeting | random | 342 |
| | | | Tggagcagatcaaacgggtgaaggactcgga | HRAS | design | |
| dz806b | CP244 | F | cttgtggaaaggacgaaacaccgAAGCCACGCCTGAGCAGAAGGA | targeting | random | 343 |
| | | | Ggtttctggcagttctcctctcctgcacccc | EGFR | design | |
| dz806b | CP244 | R | acgcacactggacgcgcaaaaaaaCTCCTTCTGCTCAGGCGTGGCT | targeting | random | 344 |
| | | | Tggggtgcaggagaggagaactgccagaaac | EGFR | design | |
| dz806b | CP245 | F | cttgtggaaaggacgaaacaccgAAGCCACGCCTGAGCAGAAGGA | targeting | random | 345 |
| | | | Gcggcctgtggcatccgcccaaacctgatgg | PPARG | design | |
| dz806b | CP245 | R | acgcacactggacgcgcaaaaaaaCTCCTTCTGCTCAGGCGTGGCT | targeting | random | 346 |
| | | | Tccatcaggtttgggcggatgccacaggccg | PPARG | design | |
| DZ806b | CP334 | F | cttgtggaaaggacgaaacaccgAAGCCACGCCTGAGCAGAAGGA | targeting | design | 347 |
| | | | Gagtttctaaacagctccacgattctctcct | STAT3 | targeting | |
| | | | | | PFS | |
| DZ806b | CP334 | R | acgcacactggacgcgcaaaaaaaCTCCTTCTGCTCAGGCGTGGCT | targeting | design | 348 |
| | | | Taggagagaatcgtggagctgtttagaaact | STAT3 | targeting | |
| | | | | | PFS | |
| DZ806b | CP335 | F | cttgtggaaaggacgaaacaccgAAGCCACGCCTGAGCAGAAGGA | targeting | design | 349 |
| | | | Gtctgacaccctgaataattcacaccaggtc | STAT3 | targeting | |
| | | | | | PFS | |
| DZ806b | CP335 | R | acgcacactggacgcgcaaaaaaaCTCCTTCTGCTCAGGCGTGGCT | targeting | design | 350 |
| | | | Tgacctggtgtgaattattcagggtgtcaga | STAT3 | targeting | |
| | | | | | PFS | |
| DZ806b | CP336 | F | cttgtggaaaggacgaaacaccgAAGCCACGCCTGAGCAGAAGGA | targeting | design | 351 |
| | | | Gagcccatgatgtacccttcgttccaaaggg | STAT3 | targeting | |
| | | | | | PFS | |
| DZ806b | CP336 | R | acgcacactggacgcgcaaaaaaaCTCCTTCTGCTCAGGCGTGGCT | targeting | design | 352 |
| | | | Tccctttggaacgaagggtacatcatgggct | STAT3 | targeting | |
| | | | | | PFS | |
| DZ806b | CP337 | F | cttgtggaaaggacgaaacaccgAAGCCACGCCTGAGCAGAAGGA | targeting | design | 353 |
| | | | Gatgaggactctaaacattgaggcttcagca | EZH2 | targeting | |
| | | | | | PFS | |
| DZ806b | CP337 | R | acgcacactggacgcgcaaaaaaaCTCCTTCTGCTCAGGCGTGGCT | targeting | design | 354 |
| | | | Ttgctgaagcctcaatgtttagagtcctcat | EZH2 | targeting | |
| | | | | | PFS | |
| DZ806b | CP338 | F | cttgtggaaaggacgaaacaccgAAGCCACGCCTGAGCAGAAGGA | targeting | design | 355 |
| | | | Ggacagaggtcagggtcacactctcggacag | EZH2 | targeting | |
| | | | | | PFS | |
| DZ806b | CP338 | R | acgcacactggacgcgcaaaaaaaCTCCTTCTGCTCAGGCGTGGCT | targeting | design | 356 |
| | | | Tagttggtgaatgcccttggtcaatataatg | EZH2 | targeting | |
| | | | | | PFS | |
| DZ806b | CP339 | F | cttgtggaaaggacgaaacaccgAAGCCACGCCTGAGCAGAAGGA | targeting | design | 357 |
| | | | Gaaatcccccagcctgccacgtcagatggtg | EZH2 | targeting | |
| | | | | | PFS | |
| DZ806b | CP339 | R | acgcacactggacgcgcaaaaaaaCTCCTTCTGCTCAGGCGTGGCT | targeting | design | 358 |
| | | | Tcaccatctgacgtggcaggctgggggattt | EZH2 | targeting | |
| | | | | | PFS | |
| DZ806b | CP340 | F | cttgtggaaaggacgaaacaccgAAGCCACGCCTGAGCAGAAGGA | targeting | design | 359 |
| | | | Gctgtgttgagggcaatgaggacataaccag | EGFR | targeting | |
| | | | | | PFS | |
| DZ806b | CP340 | R | acgcacactggacgcgcaaaaaaaCTCCTTCTGCTCAGGCGTGGCT | targeting | design | 360 |
| | | | Tctggttatgtcctcattgccctcaacacag | EGFR | targeting | |
| | | | | | PFS | |
| DZ806b | CP341 | F | cttgtggaaaggacgaaacaccgAAGCCACGCCTGAGCAGAAGGA | targeting | design | 361 |
| | | | Gtgtggcgccttcgcatgaagaggccgatcc | EGFR | targeting | |
| | | | | | PFS | |
| DZ806b | CP341 | R | acgcacactggacgcgcaaaaaaaCTCCTTCTGCTCAGGCGTGGCT | targeting | design | 362 |
| | | | Tggatcggcctcttcatgcgaaggcgccaca | EGFR | targeting | |
| | | | | | PFS | |
| DZ806b | CP342 | F | cttgtggaaaggacgaaacaccgAAGCCACGCCTGAGCAGAAGGA | targeting | design | 363 |
| | | | Gtgagctgcacggtggaggtgaggcagatgc | EGFR | targeting | |
| | | | | | PFS | |
| DZ806b | CP342 | R | acgcacactggacgcgcaaaaaaaCTCCTTCTGCTCAGGCGTGGCT | targeting | design | 364 |
| | | | Tgcatctgcctcacctccaccgtgcagctca | EGFR | targeting | |
| | | | | | PFS | |
| DZ806b | CP343 | F | cttgtggaaaggacgaaacaccgAAGCCACGCCTGAGCAGAAGGA | targeting | design | 365 |
| | | | Gcagtgcgtgcagccaggtcacacttgttcc | HRAS | targeting | |
| | | | | | PFS | |
| DZ806b | CP343 | R | acgcacactggacgcgcaaaaaaaCTCCTTCTGCTCAGGCGTGGCT | targeting | design | 366 |
| | | | Tggaacaagtgtgacctggctgcacgcactg | HRAS | targeting | |
| | | | | | PFS | |
| DZ806b | CP447 | F | cttgtggaaaggacgaaacaccgAAGCCACGCCTGAGCAGAAGGA | targeting | design | 367 |
| | | | Gcttgtgaacactggggtcgtagtcaccata | NF2 | targeting | |
| | | | | | PFS | |
| DZ806b | CP447 | R | acgcacactggacgcgcaaaaaaaCTCCTTCTGCTCAGGCGTGGCT | targeting | design | 368 |
| | | | Ttatggtgactacgaccccagtgttcacaag | NF2 | targeting | |
| | | | | | PFS | |
| DZ806b | CP448 | F | cttgtggaaaggacgaaacaccgAAGCCACGCCTGAGCAGAAGGA | targeting | design | 369 |
| | | | Gtagtcaccatacttggcctggacggcgtaa | NF2 | targeting | |
| | | | | | PFS | |
| DZ806b | CP448 | R | acgcacactggacgcgcaaaaaaaCTCCTTCTGCTCAGGCGTGGCT | targeting | design | 370 |
| | | | Tttacgccgtccaggccaagtatggtgacta | NF2 | targeting | |
| | | | | | PFS | |
| DZ806b | CP449 | F | cttgtggaaaggacgaaacaccgAAGCCACGCCTGAGCAGAAGGA | targeting | design | 371 |
| | | | Gacggcgtaagaagccaggagcacagaagcc | NF2 | targeting | |
| | | | | | PFS | |
| DZ806b | CP449 | R | acgcacactggacgcgcaaaaaaaCTCCTTCTGCTCAGGCGTGGCT | targeting | design | 372 |
| | | | Tggcttctgtgctcctggcttcttacgccgt | NF2 | targeting | |
| | | | | | PFS | |
| DZ806b | CP450 | F | cttgtggaaaggacgaaacaccgAAGCCACGCCTGAGCAGAAGGA | targeting | design | 373 |
| | | | Ggcctcggtgctctgcgtaccaagcagtaat | NF2 | targeting | |
| | | | | | PFS | |
| DZ806b | CP450 | R | acgcacactggacgcgcaaaaaaaCTCCTTCTGCTCAGGCGTGGCT | targeting | design | 374 |
| | | | Tattactgcttggtacgcagagcaccgaggc | NF2 | targeting | |
| | | | | | PFS | |
| DZ806b | CP451 | F | cttgtggaaaggacgaaacaccgAAGCCACGCCTGAGCAGAAGGA | targeting | design | 375 |
| | | | Ggcggtgggctcggtcctgcgcttgcaggtc | SMARCA4 | targeting | |
| | | | | | PFS | |
| DZ806b | CP451 | R | acgcacactggacgcgcaaaaaaaCTCCTTCTGCTCAGGCGTGGCT | targeting | design | 376 |
| | | | Tgacctgcaagcgcaggaccgagcccaccgc | SMARCA4 | targeting | |
| | | | | | PFS | |
| DZ806b | CP452 | F | cttgtggaaaggacgaaacaccgAAGCCACGCCTGAGCAGAAGGA | targeting | design | 377 |
| | | | Ggcttgcaggtcctggtgaggattccagtcg | SMARCA4 | targeting | |
| | | | | | PFS | |
| DZ806b | CP452 | R | acgcacactggacgcgcaaaaaaaCTCCTTCTGCTCAGGCGTGGCT | targeting | design | 378 |
| | | | Tcgactggaatcctcaccaggacctgcaagc | SMARCA4 | targeting | |
| | | | | | PFS | |
| DZ806b | CP453 | F | cttgtggaaaggacgaaacaccgAAGCCACGCCTGAGCAGAAGGA | targeting | design | 379 |
| | | | Gctgtcaaaaatgatcacagtgtctgccgac | SMARCA4 | targeting | |
| | | | | | PFS | |
| DZ806b | CP453 | R | acgcacactggacgcgcaaaaaaaCTCCTTCTGCTCAGGCGTGGCT | targeting | design | 380 |
| | | | Tgtcggcagacactgtgatcatttttgacag | SMARCA4 | targeting | |
| | | | | | PFS | |
| DZ806b | CP454 | F | cttgtggaaaggacgaaacaccgAAGCCACGCCTGAGCAGAAGGA | targeting | design | 381 |
| | | | Gctgcagctaggatcttctcctccacgctgt | SMARCA4 | targeting | |
| | | | | | PFS | |
| DZ806b | CP454 | R | acgcacactggacgcgcaaaaaaaCTCCTTCTGCTCAGGCGTGGCT | targeting | design | 382 |
| | | | Tacagcgtggaggagaagatcctagctgcag | SMARCA4 | targeting | |
| | | | | | PFS | |
| DZ806b | CP455 | F | cttgtggaaaggacgaaacaccgAAGCCACGCCTGAGCAGAAGGA | targeting | design | 383 |
| | | | Gcattatgagacatccccactgcaaggcatt | PPARG | targeting | |
| | | | | | PFS | |
| DZ806b | CP455 | R | acgcacactggacgcgcaaaaaaaCTCCTTCTGCTCAGGCGTGGCT | targeting | design | 384 |
| | | | Taatgccttgcagtggggatgtctcataatg | PPARG | targeting | |
| | | | | | PFS | |
| DZ806b | CP456 | F | cttgtggaaaggacgaaacaccgAAGCCACGCCTGAGCAGAAGGA | targeting | design | 385 |
| | | | Gcggcctgtggcatccgcccaaacctgatgg | PPARG | targeting | |
| | | | | | PFS | |
| DZ806b | CP456 | R | acgcacactggacgcgcaaaaaaaCTCCTTCTGCTCAGGCGTGGCT | targeting | design | 386 |
| | | | Tccatcaggtttgggcggatgccacaggccg | PPARG | targeting | |
| | | | | | PFS | |
| DZ806b | CP457 | F | cttgtggaaaggacgaaacaccgAAGCCACGCCTGAGCAGAAGGA | targeting | design | 387 |
| | | | Gatatcactggagatctccgccaacagcttc | PPARG | targeting | |
| | | | | | PFS | |
| DZ806b | CP457 | R | acgcacactggacgcgcaaaaaaaCTCCTTCTGCTCAGGCGTGGCT | targeting | design | 388 |
| | | | Tgaagctgttggcggagatctccagtgatat | PPARG | targeting | |
| | | | | | PFS | |
| DZ806b | CP458 | F | cttgtggaaaggacgaaacaccgAAGCCACGCCTGAGCAGAAGGA | targeting | design | 389 |
| | | | Gtggatccgacagttaagatcacatctgtca | PPARG | targeting | |
| | | | | | PFS | |
| DZ806b | CP458 | R | acgcacactggacgcgcaaaaaaaCTCCTTCTGCTCAGGCGTGGCT | targeting | design | 390 |
| | | | Ttgacagatgtgatcttaactgtcggatcca | PPARG | targeting | |
| | | | | | PFS | |
| DZ806b | CP459 | F | cttgtggaaaggacgaaacaccgAAGCCACGCCTGAGCAGAAGGA | targeting | design | 391 |
| | | | Gcaccagctctctgactgtacccccagagac | NFKB1 | targeting | |
| | | | | | PFS | |
| DZ806b | CP459 | R | acgcacactggacgcgcaaaaaaaCTCCTTCTGCTCAGGCGTGGCT | targeting | design | 392 |
| | | | Tgtctctgggggtacagtcagagagctggtg | NFKB1 | targeting | |
| | | | | | PFS | |
| DZ806b | CP460 | F | cttgtggaaaggacgaaacaccgAAGCCACGCCTGAGCAGAAGGA | targeting | design | 393 |
| | | | Gcccccagagacctcatagttgtccataagt | NFKB1 | targeting | |
| | | | | | PFS | |
| DZ806b | CP460 | R | acgcacactggacgcgcaaaaaaaCTCCTTCTGCTCAGGCGTGGCT | targeting | design | 394 |
| | | | Tacttatggacaactatgaggtctctggggg | NFKB1 | targeting | |
| | | | | | PFS | |
| DZ806b | CP461 | F | cttgtggaaaggacgaaacaccgAAGCCACGCCTGAGCAGAAGGA | targeting | design | 395 |
| | | | Gaaggagcaggactcagccggaaggcattat | NFKB1 | targeting | |
| | | | | | PFS | |
| DZ806b | CP461 | R | acgcacactggacgcgcaaaaaaaCTCCTTCTGCTCAGGCGTGGCT | targeting | design | 396 |
| | | | Tataatgccttccggctgagtcctgctcctt | NFKB1 | targeting | |
| | | | | | PFS | |
| DZ806b | CP462 | F | cttgtggaaaggacgaaacaccgAAGCCACGCCTGAGCAGAAGGA | targeting | design | 397 |
| | | | Gatcacttcaattgcttcggtgtagcccatt | NFKB1 | targeting | |
| | | | | | PFS | |
| DZ806b | CP462 | R | acgcacactggacgcgcaaaaaaaCTCCTTCTGCTCAGGCGTGGCT | targeting | design | 398 |
| | | | Taatgggctacaccgaagcaattgaagtgat | NFKB1 | targeting | |
| | | | | | PFS | |
| DZ806b | CP463 | F | cttgtggaaaggacgaaacaccgAAGCCACGCCTGAGCAGAAGGA | targeting | design | 399 |
| | | | Gggctgattcgctgtgacttcgaattgcatc | RAF1 | targeting | |
| | | | | | PFS | |
| DZ806b | CP463 | R | acgcacactggacgcgcaaaaaaaCTCCTTCTGCTCAGGCGTGGCT | targeting | design | 400 |
| | | | Tgatgcaattcgaagtcacagcgaatcagcc | RAF1 | targeting | |
| | | | | | PFS | |
| DZ806b | CP464 | F | cttgtggaaaggacgaaacaccgAAGCCACGCCTGAGCAGAAGGA | targeting | design | 401 |
| | | | Gcgaattgcatcctcaatcatcctgctgtcc | RAF1 | targeting | |
| | | | | | PFS | |
| DZ806b | CP464 | R | acgcacactggacgcgcaaaaaaaCTCCTTCTGCTCAGGCGTGGCT | targeting | design | 402 |
| | | | Tggacagcaggatgattgaggatgcaattcg | RAF1 | targeting | |
| | | | | | PFS | |
| DZ806b | CP465 | F | cttgtggaaaggacgaaacaccgAAGCCACGCCTGAGCAGAAGGA | targeting | design | 403 |
| | | | Gtgctgaccatgtggacattaggtgtggatg | RAF1 | targeting | |
| | | | | | PFS | |
| DZ806b | CP465 | R | acgcacactggacgcgcaaaaaaaCTCCTTCTGCTCAGGCGTGGCT | targeting | design | 404 |
| | | | Tcatccacacctaatgtccacatggtcagca | RAF1 | targeting | |
| | | | | | PFS | |
| DZ806b | CP466 | F | cttgtggaaaggacgaaacaccgAAGCCACGCCTGAGCAGAAGGA | targeting | design | 405 |
| | | | Ggctcagattgttggggctactggacagggc | RAF1 | targeting | |
| | | | | | PFS | |
| DZ806b | CP466 | R | acgcacactggacgcgcaaaaaaaCTCCTTCTGCTCAGGCGTGGCT | targeting | design | 406 |
| | | | Tgccctgtccagtagccccaacaatctgagc | RAF1 | targeting | |
| | | | | | PFS | |
| DZ806b | CP467 | F | cttgtggaaaggacgaaacaccgAAGCCACGCCTGAGCAGAAGGA | targeting | design | 407 |
| | | | Gtgatacacctcggtctcaaaggtgatcagg | STAT3 | targeting | |
| | | | | | PFS | |
| DZ806b | CP467 | R | acgcacactggacgcgcaaaaaaaCTCCTTCTGCTCAGGCGTGGCT | targeting | design | 408 |
| | | | Tcctgatcacctttgagaccgaggtgtatca | STAT3 | targeting | |
| | | | | | PFS | |
| DZ806b | CP468 | F | cttgtggaaaggacgaaacaccgAAGCCACGCCTGAGCAGAAGGA | targeting | design | 409 |
| | | | Gaattctgcagagaggctgccgttgttggat | STAT3 | targeting | |
| | | | | | PFS | |
| DZ806b | CP468 | R | acgcacactggacgcgcaaaaaaaCTCCTTCTGCTCAGGCGTGGCT | targeting | design | 410 |
| | | | Tatccaacaacggcagcctctctgcagaatt | STAT3 | targeting | |
| | | | | | PFS | |
| DZ806b | CP469 | F | cttgtggaaaggacgaaacaccgAAGCCACGCCTGAGCAGAAGGA | targeting | design | 411 |
| | | | Ggagatcaccacaactggcaaggagtgggtc | STAT3 | targeting | |
| | | | | | PFS | |
| DZ806b | CP469 | R | acgcacactggacgcgcaaaaaaaCTCCTTCTGCTCAGGCGTGGCT | targeting | design | 412 |
| | | | Tgacccactccttgccagttgtggtgatctc | STAT3 | targeting | |
| | | | | | PFS | |
| DZ806b | CP470 | F | cttgtggaaaggacgaaacaccgAAGCCACGCCTGAGCAGAAGGA | targeting | design | 413 |
| | | | Gcatctgacagatgttggagatcaccacaac | STAT3 | targeting | |
| | | | | | PFS | |
| DZ806b | CP470 | R | acgcacactggacgcgcaaaaaaaCTCCTTCTGCTCAGGCGTGGCT | targeting | design | 414 |
| | | | Tgttgtggtgatctccaacatctgtcagatg | STAT3 | targeting | |
| | | | | | PFS | |
| DZ806b | CP471 | F | cttgtggaaaggacgaaacaccgAAGCCACGCCTGAGCAGAAGGA | targeting | design | 415 |
| | | | Gacgcccaggcatttggcatctgacagatgt | STAT3 | targeting | |
| | | | | | PFS | |
| DZ806b | CP471 | R | acgcacactggacgcgcaaaaaaaCTCCTTCTGCTCAGGCGTGGCT | targeting | design | 416 |
| | | | Tacatctgtcagatgccaaatgcctgggcgt | STAT3 | targeting | |
| | | | | | PFS | |
| DZ806b | CP472 | F | cttgtggaaaggacgaaacaccgAAGCCACGCCTGAGCAGAAGGA | targeting | design | 417 |
| | | | Gatcacaattggctcggcccccattcccaca | STAT3 | targeting | |
| | | | | | PFS | |
| DZ806b | CP472 | R | acgcacactggacgcgcaaaaaaaCTCCTTCTGCTCAGGCGTGGCT | targeting | design | 418 |
| | | | Ttgtgggaatgggggccgagccaattgtgat | STAT3 | targeting | |
| | | | | | PFS | |
| DZ806b | CP473 | F | cttgtggaaaggacgaaacaccgAAGCCACGCCTGAGCAGAAGGA | targeting | design | 419 |
| | | | Gtcctcgaagttcatcacgcgctcccacttg | mCherry | targeting | |
| | | | | | PFS | |
| DZ806b | CP473 | R | acgcacactggacgcgcaaaaaaaCTCCTTCTGCTCAGGCGTGGCT | targeting | design | 420 |
| | | | Tcaagtgggagcgcgtgatgaacttcgagga | mCherry | targeting | |
| | | | | | PFS | |
| DZ806b | CP474 | F | cttgtggaaaggacgaaacaccgAAGCCACGCCTGAGCAGAAGGA | targeting | design | 421 |
| | | | Gtgcttcacgtaggccttggagccgtacatg | mCherry | targeting | |
| | | | | | PFS | |
| DZ806b | CP474 | R | acgcacactggacgcgcaaaaaaaCTCCTTCTGCTCAGGCGTGGCT | targeting | design | 422 |
| | | | Tcatgtacggctccaaggcctacgtgaagca | mCherry | targeting | |
| | | | | | PFS | |
| DZ806b | CP475 | F | cttgtggaaaggacgaaacaccgAAGCCACGCCTGAGCAGAAGGA | targeting | design | 423 |
| | | | Gaagttcatcacgcgctcccacttgaagccc | mCherry | targeting | |
| | | | | | PFS | |
| DZ806b | CP475 | R | acgcacactggacgcgcaaaaaaaCTCCTTCTGCTCAGGCGTGGCT | targeting | design | 424 |
| | | | Tgggcttcaagtgggagcgcgtgatgaactt | mCherry | targeting | |
| | | | | | PFS | |
| DZ806b | CP476 | F | cttgtggaaaggacgaaacaccgAAGCCACGCCTGAGCAGAAGGA | targeting | design | 425 |
| | | | Ggagccgtacatgaactgaggggacaggatg | mCherry | targeting | |
| | | | | | PFS | |
| DZ806b | CP476 | R | acgcacactggacgcgcaaaaaaaCTCCTTCTGCTCAGGCGTGGCT | targeting | design | 426 |
| | | | Tcatcctgtcccctcagttcatgtacggctccaaggcctacgtgaag | mCherry | targeting | |
| | | | ca | | PFS | |
| dz807 | ps1999 | F | cttgtggaaaggacgaaacaccgCGTTTCCACGGCATCACAGCCGT | targeting | random | 427 |
| | | | GGCCGAATTGAAGCcaaatgctggtaacactgtggtccacaagg | EZH2 | design | |
| dz807 | ps1999 | R | acgcacactggacgcgcaaaaaaaGCTTCAATTCGGCCACGGCTGT | targeting | random | 428 |
| | | | GATGCCGTGGAAACGccttgtggaccacagtgttaccagcatttg | EZH2 | design | |
| dz807 | ps2000 | F | cttgtggaaaggacgaaacaccgCGTTTCCACGGCATCACAGCCGT | targeting | random | 429 |
| | | | GGCCGAATTGAAGCgtgcagctcctcagtcacaatcagggaagc | STAT3 | design | |
| dz807 | ps2000 | R | acgcacactggacgcgcaaaaaaaGCTTCAATTCGGCCACGGCTGT | targeting | random | 430 |
| | | | GATGCCGTGGAAACGgcttccctgattgtgactgaggagctgcac | STAT3 | design | |
| dz809 | ps2003 | F | cttgtggaaaggacgaaacaccgGTAAGAATCAAATAATCCCGAT | targeting | random | 431 |
| | | | ACGCGGGATTAAGACcaaatgctggtaacactgtggtccacaagg | EZH2 | design | |
| dz809 | ps2003 | R | acgcacactggacgcgcaaaaaaaGTCTTAATCCCGCGTATCGGGA | targeting | random | 432 |
| | | | TTATTTGATTCTTACccttgtggaccacagtgttaccagcatttg | EZH2 | design | |
| dz809 | ps2004 | F | cttgtggaaaggacgaaacaccgGTAAGAATCAAATAATCCCGAT | targeting | random | 433 |
| | | | ACGCGGGATTAAGACgtgcagctcctcagtcacaatcagggaagc | STAT3 | design | |
| dz809 | ps2004 | R | acgcacactggacgcgcaaaaaaaGTCTTAATCCCGCGTATCGGGA | targeting | random | 434 |
| | | | TTATTTGATTCTTACgcttccctgattgtgactgaggagctgcac | STAT3 | design | |
| dz810 | ps2005 | F | cttgtggaaaggacgaaacaccgGCTGCATTCCCCGCGCGAGAGG | targeting | random | 435 |
| | | | GGATTGAGACcaaatgctggtaacactgtggtccacaagg | EZH2 | design | |
| dz810 | ps2005 | R | acgcacactggacgcgcaaaaaaaGTCTCAATCCCCTCTCGCGCGG | targeting | random | 436 |
| | | | GGAATGCAGCccttgtggaccacagtgttaccagcatttg | EZH2 | design | |
| dz810 | ps2006 | F | cttgtggaaaggacgaaacaccgGCTGCATTCCCCGCGCGAGAGG | targeting | random | 437 |
| | | | GGATTGAGACgtgcagctcctcagtcacaatcagggaagc | STAT3 | design | |
| dz810 | ps2006 | R | acgcacactggacgcgcaaaaaaaGTCTCAATCCCCTCTCGCGCGG | targeting | random | 438 |
| | | | GGAATGCAGCgcttccctgattgtgactgaggagctgcac | STAT3 | design | |
| dz811 | ps2007 | F | cttgtggaaaggacgaaacaccgGTTGTGTGTACCCTTCGAATAGA | targeting | random | 439 |
| | | | GGGTAGATCCAACcaaatgctggtaacactgtggtccacaagg | EZH2 | design | |
| dz811 | ps2007 | R | acgcacactggacgcgcaaaaaaaGTTGGATCTACCCTCTATTCGA | targeting | random | 440 |
| | | | AGGGTACACACAACccttgtggaccacagtgttaccagcatttg | EZH2 | design | |
| dz811 | ps2008 | F | cttgtggaaaggacgaaacaccgGTTGTGTGTACCCTTCGAATAGA | targeting | random | 441 |
| | | | GGGTAGATCCAACgtgcagctcctcagtcacaatcagggaagc | STAT3 | design | |
| dz811 | ps2008 | R | acgcacactggacgcgcaaaaaaaGTTGGATCTACCCTCTATTCGA | targeting | random | 442 |
| | | | AGGGTACACACAACgcttccctgattgtgactgaggagctgcac | STAT3 | design | |
| dz812 | ps2009 | F | cttgtggaaaggacgaaacaccgGTCGCGCCTTCGCGGGCGCGTG | targeting | random | 443 |
| | | | AGTTGAAACcaaatgctggtaacactgtggtccacaagg | EZH2 | design | |
| dz812 | ps2009 | R | acgcacactggacgcgcaaaaaaaGTTTCAACTCACGCGCCCGCGA | targeting | random | 444 |
| | | | AGGCGCGACccttgtggaccacagtgttaccagcatttg | EZH2 | design | |
| dz812 | ps2010 | F | cttgtggaaaggacgaaacaccgGTCGCGCCTTCGCGGGCGCGTG | targeting | random | 445 |
| | | | AGTTGAAACgtgcagctcctcagtcacaatcagggaagc | STAT3 | design | |
| dz812 | ps2010 | R | acgcacactggacgcgcaaaaaaaGTTTCAACTCACGCGCCCGCGA | targeting | random | 446 |
| | | | AGGCGCGACgcttccctgattgtgactgaggagctgcac | STAT3 | design | |
| dz813 | ps2011 | F | cttgtggaaaggacgaaacaccgGGTTCCCCCGTACACGCGGGGA | targeting | random | 447 |
| | | | TAGACCcaaatgctggtaacactgtggtccacaagg | EZH2 | design | |
| dz813 | ps2011 | R | acgcacactggacgcgcaaaaaaaGGTCTATCCCCGCGTGTACGGG | targeting | random | 448 |
| | | | GGAACCccttgtggaccacagtgttaccagcatttg | EZH2 | design | |
| dz813 | ps2012 | F | cttgtggaaaggacgaaacaccgGGTTCCCCCGTACACGCGGGGA | targeting | random | 449 |
| | | | TAGACCgtgcagctcctcagtcacaatcagggaagc | STAT3 | design | |
| dz813 | ps2012 | R | acgcacactggacgcgcaaaaaaaGGTCTATCCCCGCGTGTACGGG | targeting | random | 450 |
| | | | GGAACCgcttccctgattgtgactgaggagctgcac | STAT3 | design | |
| dz814 | ps2035 | F | cttgtggaaaggacgaaacaccgGTGCTCCCCGCACACGCGGGGA | targeting | random | 451 |
| | | | TGATCCCCAAATGCTGGTAACACTGTGGTCCACAAGG | EZH2 | design | |
| dz814 | ps2035 | R | ACgcacactggacgcgcAAAAAAAGGGATCATCCCCGCGTGT | targeting | random | 452 |
| | | | GCGGGGAGCACCCTTGTGGACCACAGTGTTACCAGCA | EZH2 | design | |
| | | | TTTG | | | |
| dz814 | ps2036 | F | cttgtggaaaggacgaaacaccgGTGCTCCCCGCACACGCGGGGA | targeting | random | 453 |
| | | | TGATCCCGTGCAGCTCCTCAGTCACAATCAGGGAAGC | STAT3 | design | |
| dz814 | ps2036 | R | ACgcacactggacgcgcAAAAAAAGGGATCATCCCCGCGTGT | targeting | random | 454 |
| | | | GCGGGGAGCACGCTTCCCTGATTGTGACTGAGGAGCT | STAT3 | design | |
| | | | GCAC | | | |
| dz815 | ps2037 | F | cttgtggaaaggacgaaacaccgGGTGGAGACACGCGGATTTAGG | targeting | random | 455 |
| | | | GGTGTGATGACAGGCAAATGCTGGTAACACTGTGGTC | EZH2 | design | |
| | | | CACAAGG | | | |
| dz815 | ps2037 | R | ACgcacactggacgcgcAAAAAAACCTGTCATCACACCCCTA | targeting | random | 456 |
| | | | AATCCGCGTGTCTCCACCCCTTGTGGACCACAGTGTTA | EZH2 | design | |
| | | | CCAGCATTTG | | | |
| dz815 | ps2038 | F | cttgtggaaaggacgaaacaccgGGTGGAGACACGCGGATTTAGG | targeting | random | 457 |
| | | | GGTGTGATGACAGGGTGCAGCTCCTCAGTCACAATCA | STAT3 | \|design | |
| | | | GGGAAGC | | | |
| dz815 | ps2038 | R | ACgcacactggacgcgcAAAAAAACCTGTCATCACACCCCTA | targeting | random | 458 |
| | | | AATCCGCGTGTCTCCACCGCTTCCCTGATTGTGACTGA | STAT3 | design | |
| | | | GGAGCTGCAC | | | |
| dz816a | ps2013 | F | cttgtggaaaggacgaaacaccgATTCCTAAGCTCTTACGCTTAGG | targeting | random | 459 |
| | | | ACTTCATTGAGGcaaatgctggtaacactgtggtccacaagg | EZH2 | design | |
| dz816a | ps2013 | R | acgcacactggacgcgcaaaaaaaCCTCAATGAAGTCCTAAGCGT | targeting | random | 460 |
| | | | AAGAGCTTAGGAATccttgtggaccacagtgttaccagcatttg | EZH2 | design | |
| dz816a | ps2014 | F | cttgtggaaaggacgaaacaccgATTCCTAAGCTCTTACGCTTAGG | targeting | random | 461 |
| | | | ACTTCATTGAGGgtgcagctcctcagtcacaatcagggaagc | STAT3 | design | |
| dz816a | ps2014 | R | acgcacactggacgcgcaaaaaaaCCTCAATGAAGTCCTAAGCGT | targeting | random | 462 |
| | | | AAGAGCTTAGGAATgcttccctgattgtgactgaggagctgcac | STAT3 | design | |
| dz816b | ps2015 | F | cttgtggaaaggacgaaacaccgCCTCAATGAAGTCCTAAGCGTA | targeting | random | 463 |
| | | | AAAGCTTAGGAATcaaatgctggtaacactgtggtccacaagg | EZH2 | design | |
| dz816b | ps2015 | R | acgcacactggacgcgcaaaaaaaATTCCTAAGCTTTTACGCTTAG | targeting | random | 464 |
| | | | GACTTCATTGAGGccttgtggaccacagtgttaccagcatttg | EZH2 | design | |
| dz816b | ps2016 | F | cttgtggaaaggacgaaacaccgCCTCAATGAAGTCCTAAGCGTA | targeting | random | 465 |
| | | | AAAGCTTAGGAATgtgcagctcctcagtcacaatcagggaagc | STAT3 | design | |
| dz816b | ps2016 | R | acgcacactggacgcgcaaaaaaaATTCCTAAGCTTTTACGCTTAG | targeting | random | 466 |
| | | | GACTTCATTGAGGgcttccctgattgtgactgaggagctgcac | STAT3 | design | |
| dz817a | ps2017 | F | cttgtggaaaggacgaaacaccgCCCTCAACTATTGAAACGTGTTT | targeting | random | 467 |
| | | | CAGTCGTTTCAGGcaaatgctggtaacactgtggtccacaagg | EZH2 | design | |
| dz817a | ps2017 | R | acgcacactggacgcgcaaaaaaaCCTGAAACGACTGAAACACGT | targeting | random | 468 |
| | | | TTCAATAGTTGAGGGccttgtggaccacagtgttaccagcatttg | EZH2 | design | |
| dz817a | ps2018 | F | cttgtggaaaggacgaaacaccgCCCTCAACTATTGAAACGTGTTT | targeting | random | 469 |
| | | | CAGTCGTTTCAGGgtgcagctcctcagtcacaatcagggaagc | STAT3 | design | |
| dz817a | ps2018 | R | acgcacactggacgcgcaaaaaaaCCTGAAACGACTGAAACACGT | targeting | random | 470 |
| | | | TTCAATAGTTGAGGGgcttccctgattgtgactgaggagctgcac | STAT3 | design | |
| dz817b | ps2019 | F | cttgtggaaaggacgaaacaccgCCTGAAACGACTGAAACACGTT | targeting | random | 471 |
| | | | TCAATAGTTGAGGGcaaatgctggtaacactgtggtccacaagg | EZH2 | design | |
| dz817b | ps2019 | R | acgcacactggacgcgcaaaaaaaCCCTCAACTATTGAAACGTGTT | targeting | random | 472 |
| | | | TCAGTCGTTTCAGGccttgtggaccacagtgttaccagcatttg | EZH2 | design | |
| dz817b | ps2020 | F | cttgtggaaaggacgaaacaccgCCTGAAACGACTGAAACACGTT | targeting | random | 473 |
| | | | TCAATAGTTGAGGGgtgcagctcctcagtcacaatcagggaagc | STAT3 | design | |
| dz817b | ps2020 | R | acgcacactggacgcgcaaaaaaaCCCTCAACTATTGAAACGTGTT | targeting | random | 474 |
| | | | TCAGTCGTTTCAGGgcttccctgattgtgactgaggagctgcac | STAT3 | design | |
| dz819 | ps2039 | F | cttgtggaaaggacgaaacaccgGGTTTCCGTCCCCGTGAAGGGG | targeting | random | 475 |
| | | | AAGTTGTATGAAACCAAATGCTGGTAACACTGTGGTC | EZH2 | design | |
| | | | CACAAGG | | | |
| dz819 | ps2039 | R | ACgcacactggacgcgcAAAAAAAGTTTCATACAACTTCCCC | targeting | random | 476 |
| | | | TTCACGGGGACGGAAACCCCTTGTGGACCACAGTGTT | EZH2 | design | |
| | | | ACCAGCATTTG | | | |
| dz819 | ps2040 | F | cttgtggaaaggacgaaacaccgGGTTTCCGTCCCCGTGAAGGGG | targeting | random | 477 |
| | | | AAGTTGTATGAAACGTGCAGCTCCTCAGTCACAATCA | STAT3 | design | |
| | | | GGGAAGC | | | |
| dz819 | ps2040 | R | ACgcacactggacgcgcAAAAAAAGTTTCATACAACTTCCCC | targeting | random | 478 |
| | | | TTCACGGGGACGGAAACCGCTTCCCTGATTGTGACTG | STAT3 | design | |
| | | | AGGAGCTGCAC | | | |
| dz820 | ps2023 | F | cttgtggaaaggacgaaacaccgTTATGTGCTCAGGGCCACTGCAT | targeting | random | 479 |
| | | | GGTGCTGATGGAGGCCACcaaatgctggtaacactgtggtccacaagg | EZH2 | design | |
| dz820 | ps2023 | R | acgcacactggacgcgcaaaaaaaGTGGCCTCCATCAGCACCATGC | targeting | random | 480 |
| | | | AGTGGCCCTGAGCACATAAccttgtggaccacagtgttaccagcat | EZH2 | design | |
| | | | ttg | | | |
| dz820 | ps2024 | F | cttgtggaaaggacgaaacaccgTTATGTGCTCAGGGCCACTGCAT | targeting | random | 481 |
| | | | GGTGCTGATGGAGGCCACgtgcagctcctcagtcacaatcagggaagc | STAT3 | design | |
| dz820 | ps2024 | R | acgcacactggacgcgcaaaaaaaGTGGCCTCCATCAGCACCATGC | targeting | random | 482 |
| | | | AGTGGCCCTGAGCACATAAgcttccctgattgtgactgaggagctg | STAT3 | design | |
| | | | cac | | | |
| dz821 | ps2025 | F | cttgtggaaaggacgaaacaccgGGTGTCGGAAACCGCTAATTCA | targeting | random | 483 |
| | | | GGGGCCGCTACAACcaaatgctggtaacactgtggtccacaagg | EZH2 | design | |
| dz821 | ps2025 | R | acgcacactggacgcgcaaaaaaaGTTGTAGCGGCCCCTGAATTAG | targeting | random | 484 |
| | | | CGGTTTCCGACACCccttgtggaccacagtgttaccagcatttg | EZH2 | design | |
| dz821 | ps2026 | F | cttgtggaaaggacgaaacaccgGGTGTCGGAAACCGCTAATTCA | targeting | random | 485 |
| | | | GGGGCCGCTACAACgtgcagctcctcagtcacaatcagggaagc | STAT3 | design | |
| dz821 | ps2026 | R | acgcacactggacgcgcaaaaaaaGTTGTAGCGGCCCCTGAATTAG | targeting | random | 486 |
| | | | CGGTTTCCGACACCgcttccctgattgtgactgaggagctgcac | STAT3 | design | |
| dz821 | CP246 | F | cttgtggaaaggacgaaacaccgGGTGTCGGAAACCGCTAATTCA | targeting | random | 487 |
| | | | GGGGCCGCTACAACgcggtgggctcggtcctgcgcttgcaggtc | SMARCA4 | design | |
| dz821 | CP246 | R | acgcacactggacgcgcaaaaaaaGTTGTAGCGGCCCCTGAATTAG | targeting | random | 488 |
| | | | CGGTTTCCGACACCgacctgcaagcgcaggaccgagcccaccgc | SMARCA4 | design | |
| dz821 | CP247 | F | cttgtggaaaggacgaaacaccgGGTGTCGGAAACCGCTAATTCA | targeting | random | 489 |
| | | | GGGGCCGCTACAACtccgagtccttcacccgtttgatctgctcc | HRAS | design | |
| dz821 | CP247 | R | acgcacactggacgcgcaaaaaaaGTTGTAGCGGCCCCTGAATTAG | targeting | random | 490 |
| | | | CGGTTTCCGACACCggagcagatcaaacgggtgaaggactcgga | HRAS | design | |
| dz821 | CP248 | F | cttgtggaaaggacgaaacaccgGGTGTCGGAAACCGCTAATTCA | targeting | random | 491 |
| | | | GGGGCCGCTACAACgtttctggcagttctcctctcctgcacccc | EGFR | design | |
| dz821 | CP248 | R | acgcacactggacgcgcaaaaaaaGTTGTAGCGGCCCCTGAATTAG | targeting | random | 492 |
| | | | CGGTTTCCGACACCggggtgcaggagaggagaactgccagaaac | EGFR | design | |
| dz821 | CP249 | F | cttgtggaaaggacgaaacaccgGGTGTCGGAAACCGCTAATTCA | targeting | random | 493 |
| | | | GGGGCCGCTACAACcggcctgtggcatccgcccaaacctgatgg | PPARG | design | |
| dz821 | CP249 | R | acgcacactggacgcgcaaaaaaaGTTGTAGCGGCCCCTGAATTAG | targeting | random | 494 |
| | | | CGGTTTCCGACACCccatcaggtttgggcggatgccacaggccg | PPARG | design | |
| dz822 | ps2041 | F | cttgtggaaaggacgaaacaccgAGTTTAGCAGATTGGGATTTGTA | targeting | random | 495 |
| | | | CTCTGACCGGAACCAAATGCTGGTAACACTGTGGTCC | EZH2 | design | |
| | | | ACAAGG | | | |
| dz822 | ps2041 | R | ACgcacactggacgcgcAAAAAAAGTTCCGGTCAGAGTACAA | targeting | random | 496 |
| | | | ATCCCAATCTGCTAAACTCCTTGTGGACCACAGTGTTA | EZH2 | design | |
| | | | CCAGCATTTG | | | |
| dz822 | ps2042 | F | cttgtggaaaggacgaaacaccgAGTTTAGCAGATTGGGATTTGTA | targeting | random | 497 |
| | | | CTCTGACCGGAACGTGCAGCTCCTCAGTCACAATCAG | STAT3 | design | |
| | | | GGAAGC | | | |
| dz822 | ps2042 | R | ACgcacactggacgcgcAAAAAAAGTTCCGGTCAGAGTACAA | targeting | random | 498 |
| | | | ATCCCAATCTGCTAAACTGCTTCCCTGATTGTGACTGA | STAT3 | design | |
| | | | GGAGCTGCAC | | | |
| dz822 | CP250 | F | cttgtggaaaggacgaaacaccgAGTTTAGCAGATTGGGATTTGTA | targeting | random | 499 |
| | | | CTCTGACCGGAACgcggtgggctcggtcctgcgcttgcaggtc | SMARCA4 | design | |
| dz822 | CP250 | R | acgcacactggacgcgcaaaaaaaGTTCCGGTCAGAGTACAAATC | targeting | random | 500 |
| | | | CCAATCTGCTAAACTgacctgcaagcgcaggaccgagcccaccgc | SMARCA4 | design | |
| dz822 | CP251 | F | cttgtggaaaggacgaaacaccgAGTTTAGCAGATTGGGATTTGTA | targeting | random | 501 |
| | | | CTCTGACCGGAACtccgagtccttcacccgtttgatctgctcc | HRAS | design | |
| dz822 | CP251 | R | acgcacactggacgcgcaaaaaaaGTTCCGGTCAGAGTACAAATC | targeting | random | 502 |
| | | | CCAATCTGCTAAACTggagcagatcaaacgggtgaaggactcgga | HRAS | design | |
| dz822 | CP252 | F | cttgtggaaaggacgaaacaccgAGTTTAGCAGATTGGGATTTGTA | targeting | random | 503 |
| | | | CTCTGACCGGAACgtttctggcagttctcctctcctgcacccc | EGFR | design | |
| dz822 | CP252 | R | acgcacactggacgcgcaaaaaaaGTTCCGGTCAGAGTACAAATC | targeting | random | 504 |
| | | | CCAATCTGCTAAACTggggtgcaggagaggagaactgccagaaac | EGFR | design | |
| dz822 | CP253 | F | cttgtggaaaggacgaaacaccgAGTTTAGCAGATTGGGATTTGTA | targeting | random | 505 |
| | | | CTCTGACCGGAACcggcctgtggcatccgcccaaacctgatgg | PPARG | design | |
| dz822 | CP253 | R | acgcacactggacgcgcaaaaaaaGTTCCGGTCAGAGTACAAATC | targeting | random | 506 |
| | | | CCAATCTGCTAAACTccatcaggtttgggggatgccacaggccg | PPARG | design | |
| DZ822 | CP346 | F | cttgtggaaaggacgaaacaccgAGTTTAGCAGATTGGGATTTGTA | targeting | design | 507 |
| | | | CTCTGACCGGAACtcttccggacatcctgaaggtgctgctcca | STAT3 | targeting | |
| | | | | | PFS | |
| DZ822 | CP346 | R | acgcacactggacgcgcaaaaaaaGTTCCGGTCAGAGTACAAATC | targeting | design | 508 |
| | | | CCAATCTGCTAAACTtggagcagcaccttcaggatgtccggaaga | STAT3 | targeting | |
| | | | | | PFS | |
| DZ822 | CP347 | F | cttgtggaaaggacgaaacaccgAGTTTAGCAGATTGGGATTTGTA | targeting | design | 509 |
| | | | CTCTGACCGGAACtccaatgcaggcaatctgttgccgcctctt | STAT3 | targeting | |
| | | | | | PFS | |
| DZ822 | CP347 | R | acgcacactggacgcgcaaaaaaaGTTCCGGTCAGAGTACAAATC | targeting | design | 510 |
| | | | CCAATCTGCTAAACTaagaggcggcaacagattgcctgcattgga | STAT3 | targeting | |
| | | | | | PFS | |
| DZ822 | CP348 | F | cttgtggaaaggacgaaacaccgAGTTTAGCAGATTGGGATTTGTA | targeting | design | 511 |
| | | | CTCTGACCGGAACcttggtgatacacctcggtctcaaaggtga | STAT3 | targeting | |
| | | | | | PFS | |
| DZ822 | CP348 | R | acgcacactggacgcgcaaaaaaaGTTCCGGTCAGAGTACAAATC | targeting | design | 512 |
| | | | CCAATCTGCTAAACTtcacctttgagaccgaggtgtatcaccaag | STAT3 | targeting | |
| | | | | | PFS | |
| DZ822 | CP349 | F | cttgtggaaaggacgaaacaccgAGTTTAGCAGATTGGGATTTGTA | targeting | design | 513 |
| | | | CTCTGACCGGAACcaagaatacattatgggtactgaagcaact | EZH2 | targeting | |
| | | | | | PFS | |
| DZ822 | CP349 | R | acgcacactggacgcgcaaaaaaaGTTCCGGTCAGAGTACAAATC | targeting | design | 514 |
| | | | CCAATCTGCTAAACTagttgcttcagtacccataatgtattcttg | EZH2 | targeting | |
| | | | | | PFS | |
| DZ822 | CP350 | F | cttgtggaaaggacgaaacaccgAGTTTAGCAGATTGGGATTTGTA | targeting | design | 515 |
| | | | CTCTGACCGGAACgtttcagtccctgcttccctatcactgtct | EZH2 | targeting | |
| | | | | | PFS | |
| DZ822 | CP350 | R | acgcacactggacgcgcaaaaaaaGTTCCGGTCAGAGTACAAATC | targeting | design | 516 |
| | | | CCAATCTGCTAAACTagacagtgatagggaagcagggactgaaac | EZH2 | targeting | |
| | | | | | PFS | |
| DZ822 | CP351 | F | cttgtggaaaggacgaaacaccgAGTTTAGCAGATTGGGATTTGTA | targeting | design | 517 |
| | | | CTCTGACCGGAACtgccgtggatgatcacagggttgatagttg | EZH2 | targeting | |
| | | | | | PFS | |
| DZ822 | CP351 | R | acgcacactggacgcgcaaaaaaaGTTCCGGTCAGAGTACAAATC | targeting | design | 518 |
| | | | CCAATCTGCTAAACTcaactatcaaccctgtgatcatccacggca | EZH2 | targeting | |
| | | | | | PFS | |
| DZ822 | CP352 | F | cttgtggaaaggacgaaacaccgAGTTTAGCAGATTGGGATTTGTA | targeting | design | 519 |
| | | | CTCTGACCGGAACtccactgtgttgagggcaatgaggacataa | EGFR | targeting | |
| | | | | | PFS | |
| DZ822 | CP352 | R | acgcacactggacgcgcaaaaaaaGTTCCGGTCAGAGTACAAATC | targeting | design | 520 |
| | | | CCAATCTGCTAAACTttatgtcctcattgccctcaacacagtgga | EGFR | targeting | |
| | | | | | PFS | |
| DZ822 | CP353 | F | cttgtggaaaggacgaaacaccgAGTTTAGCAGATTGGGATTTGTA | targeting | design | 521 |
| | | | CTCTGACCGGAACtggttgtggcagcagtcactgggggacttg | EGFR | targeting | |
| | | | | | PFS | |
| DZ822 | CP353 | R | acgcacactggacgcgcaaaaaaaGTTCCGGTCAGAGTACAAATC | targeting | design | 522 |
| | | | CCAATCTGCTAAACTcaagtcccccagtgactgctgccacaacca | EGFR | targeting | |
| | | | | | PFS | |
| DZ822 | CP354 | F | cttgtggaaaggacgaaacaccgAGTTTAGCAGATTGGGATTTGTA | targeting | design | 523 |
| | | | CTCTGACCGGAACctaaatgccaccggcaggatgtggagatcg | EGFR | targeting | |
| | | | | | PFS | |
| DZ822 | CP354 | R | acgcacactggacgcgcaaaaaaaGTTCCGGTCAGAGTACAAATC | targeting | design | 524 |
| | | | CCAATCTGCTAAACTcgatctccacatcctgccggtggcatttag | EGFR | targeting | |
| | | | | | PFS | |
| DZ822 | CP355 | F | cttgtggaaaggacgaaacaccgAGTTTAGCAGATTGGGATTTGTA | targeting | design | 525 |
| | | | CTCTGACCGGAACtggatctgttcttgtgaatggaatgtcttc | PPARG | targeting | |
| | | | | | PFS | |
| DZ822 | CP355 | R | acgcacactggacgcgcaaaaaaaGTTCCGGTCAGAGTACAAATC | targeting | design | 526 |
| | | | CCAATCTGCTAAACTgaagacattccattcacaagaacagatcca | PPARG | targeting | |
| | | | | | PFS | |
| DZ822 | CP356 | F | cttgtggaaaggacgaaacaccgAGTTTAGCAGATTGGGATTTGTA | targeting | design | 527 |
| | | | CTCTGACCGGAACactgcaaggcatttctgaaaccgacagtac | PPARG | targeting | |
| | | | | | PFS | |
| DZ822 | CP356 | R | acgcacactggacgcgcaaaaaaaGTTCCGGTCAGAGTACAAATC | targeting | design | 528 |
| | | | CCAATCTGCTAAACTgtactgtcggtttcagaaatgccttgcagt | PPARG | targeting | |
| | | | | | PFS | |
| DZ822 | CP357 | F | cttgtggaaaggacgaaacaccgAGTTTAGCAGATTGGGATTTGTA | targeting | design | 529 |
| | | | CTCTGACCGGAACtccatatttgaggagagttacttggtcgtt | PPARG | targeting | |
| | | | | | PFS | |
| DZ822 | CP357 | R | acgcacactggacgcgcaaaaaaaGTTCCGGTCAGAGTACAAATC | targeting | design | 530 |
| | | | CCAATCTGCTAAACTaacgaccaagtaactctcctcaaatatgga | PPARG | targeting | |
| | | | | | PFS | |
| dz824 | ps2027 | F | cttgtggaaaggacgaaacaccgGTAGAAATGAGTACAAAGCGAT | targeting | random | 531 |
| | | | AGAGAGCTTAATAACcaaatgctggtaacactgtggtccacaagg | EZH2 | design | |
| dz824 | ps2027 | R | acgcacactggacgcgcaaaaaaaGTTATTAAGCTCTCTATCGCTT | targeting | random | 532 |
| | | | TGTACTCATTTCTACccttgtggaccacagtgttaccagcatttg | EZH2 | design | |
| dz824 | ps2028 | F | cttgtggaaaggacgaaacaccgGTAGAAATGAGTACAAAGCGAT | targeting | random | 533 |
| | | | AGAGAGCTTAATAACgtgcagctcctcagtcacaatcagggaagc | STAT3 | design | |
| dz824 | ps2028 | R | acgcacactggacgcgcaaaaaaaGTTATTAAGCTCTCTATCGCTT | targeting | random | 534 |
| | | | TGTACTCATTTCTACgcttccctgattgtgactgaggagctgcac | STAT3 | design | |
| dz825a | ps2043 | F | cttgtggaaaggacgaaacaccgAACTCGGAAGGATTCAGAAGAA | targeting | random | 535 |
| | | | GCTTTCATCTCAAATGCTGGTAACACTGTGGTCCACAA | EZH2 | design | |
| | | | GG | | | |
| dz825a | ps2043 | R | ACgcacactggacgcgcAAAAAAAAGATGAAAGCTTCTTCTG | targeting | random | 536 |
| | | | AATCCTTCCGAGTTCCTTGTGGACCACAGTGTTACCAG | EZH2 | design | |
| | | | CATTTG | | | |
| dz825a | ps2044 | F | cttgtggaaaggacgaaacaccgAACTCGGAAGGATTCAGAAGAA | targeting | random | 537 |
| | | | GCTTTCATCTGTGCAGCTCCTCAGTCACAATCAGGGAA | STAT3 | design | |
| | | | GC | | | |
| dz825a | ps2044 | R | ACgcacactggacgcgcAAAAAAAAGATGAAAGCTTCTTCTG | targeting | random | 538 |
| | | | AATCCTTCCGAGTTGCTTCCCTGATTGTGACTGAGGAG | STAT3 | design | |
| | | | CTGCAC | | | |
| dz825a | CP254 | F | cttgtggaaaggacgaaacaccgAACTCGGAAGGATTCAGAAGAA | targeting | random | 539 |
| | | | GCTTTCATCTgcggtgggctcggtcctgcgcttgcaggtc | SMARCA4 | design | |
| dz825a | CP254 | R | acgcacactggacgcgcaaaaaaaAGATGAAAGCTTCTTCTGAATC | targeting | random | 540 |
| | | | CTTCCGAGTTgacctgcaagcgcaggaccgagcccaccgc | SMARCA4 | design | |
| dz825a | CP255 | F | cttgtggaaaggacgaaacaccgAACTCGGAAGGATTCAGAAGAA | targeting | random | 541 |
| | | | GCTTTCATCTtccgagtccttcacccgtttgatctgctcc | HRAS | design | |
| dz825a | CP255 | R | acgcacactggacgcgcaaaaaaaAGATGAAAGCTTCTTCTGAATC | targeting | random | 542 |
| | | | CTTCCGAGTTggagcagatcaaacgggtgaaggactcgga | HRAS | design | |
| dz825a | CP256 | F | cttgtggaaaggacgaaacaccgAACTCGGAAGGATTCAGAAGAA | targeting | random | 543 |
| | | | GCTTTCATCTgtttctggcagttctcctctcctgcacccc | EGFR | design | |
| dz825a | CP256 | R | acgcacactggacgcgcaaaaaaaAGATGAAAGCTTCTTCTGAATC | targeting | random | 544 |
| | | | CTTCCGAGTTggggtgcaggagaggagaactgccagaaac | EGFR | design | |
| dz825a | CP257 | F | cttgtggaaaggacgaaacaccgAACTCGGAAGGATTCAGAAGAA | targeting | random | 545 |
| | | | GCTTTCATCTcggcctgtggcatccgcccaaacctgatgg | PPARG | design | |
| dz825a | CP257 | R | acgcacactggacgcgcaaaaaaaAGATGAAAGCTTCTTCTGAATC | targeting | random | 546 |
| | | | CTTCCGAGTTccatcaggtttgggcggatgccacaggccg | PPARG | design | |
| DZ825a | CP312 | F | cttgtggaaaggacgaaacaccgAACTCGGAAGGATTCAGAAGAA | targeting | design | 547 |
| | | | GCTTTCATCTccaggagattatgaaacaccaaagtggcat | STAT3 | targeting | |
| | | | | | PFS | |
| DZ825a | CP312 | R | acgcacactggacgcgcaaaaaaaAGATGAAAGCTTCTTCTGAATC | targeting | design | 548 |
| | | | CTTCCGAGTTatgccactttggtgtttcataatctcctgg | STAT3 | targeting | |
| | | | | | PFS | |
| DZ825a | CP313 | F | cttgtggaaaggacgaaacaccgAACTCGGAAGGATTCAGAAGAA | targeting | design | 549 |
| | | | GCTTTCATCTggacatcctgaaggtgctgctccagcatct | STAT3 | targeting | |
| | | | | | PFS | |
| DZ825a | CP313 | R | acgcacactggacgcgcaaaaaaaAGATGAAAGCTTCTTCTGAATC | targeting | design | 550 |
| | | | CTTCCGAGTTagatgctggagcagcaccttcaggatgtcc | STAT3 | targeting | |
| | | | | | PFS | |
| DZ825a | CP314 | F | cttgtggaaaggacgaaacaccgAACTCGGAAGGATTCAGAAGAA | targeting | design | 551 |
| | | | GCTTTCATCTaatgcaggcaatctgttgccgcctcttcca | STAT3 | targeting | |
| | | | | | PFS | |
| DZ825a | CP314 | R | acgcacactggacgcgcaaaaaaaAGATGAAAGCTTCTTCTGAATC | targeting | design | 552 |
| | | | CTTCCGAGTTtggaagaggcggcaacagattgcctgcatt | STAT3 | targeting | |
| | | | | | PFS | |
| DZ825a | CP315 | F | cttgtggaaaggacgaaacaccgAACTCGGAAGGATTCAGAAGAA | targeting | design | 553 |
| | | | GCTTTCATCTtgctgtaggggagaccaagaatacattatg | EZH2 | targeting | |
| | | | | | PFS | |
| DZ825a | CP315 | R | acgcacactggacgcgcaaaaaaaAGATGAAAGCTTCTTCTGAATC | targeting | design | 554 |
| | | | CTTCCGAGTTcataatgtattcttggtctcccctacagca | EZH2 | targeting | |
| | | | | | PFS | |
| DZ825a | CP316 | F | cttgtggaaaggacgaaacaccgAACTCGGAAGGATTCAGAAGAA | targeting | design | 555 |
| | | | GCTTTCATCTttctgctgtgcccttatctggaaacattga | EZH2 | targeting | |
| | | | | | PFS | |
| DZ825a | CP316 | R | acgcacactggacgcgcaaaaaaaAGATGAAAGCTTCTTCTGAATC | targeting | design | 556 |
| | | | CTTCCGAGTTtcaatgtttccagataagggcacagcagaa | EZH2 | targeting | |
| | | | | | PFS | |
| DZ825a | CP317 | F | cttgtggaaaggacgaaacaccgAACTCGGAAGGATTCAGAAGAA | targeting | design | 557 |
| | | | GCTTTCATCTtactgttattgggaagccgtcctcttctgc | EZH2 | targeting | |
| | | | | | PFS | |
| DZ825a | CP317 | R | acgcacactggacgcgcaaaaaaaAGATGAAAGCTTCTTCTGAATC | targeting | design | 558 |
| | | | CTTCCGAGTTgcagaagaggacggcttcccaataacagta | EZH2 | targeting | |
| | | | | | PFS | |
| dz825b | ps2045 | F | cttgtggaaaggacgaaacaccgAGATGAAAGCTTCTTCTGAATCC | targeting | random | 559 |
| | | | TTCCGAGTTCAAATGCTGGTAACACTGTGGTCCACAA | EZH2 | design | |
| | | | GG | | | |
| dz825b | ps2045 | R | ACgcacactggacgcgcAAAAAAAAACTCGGAAGGATTCAGA | targeting | random | 560 |
| | | | AGAAGCTTTCATCTCCTTGTGGACCACAGTGTTACCAG | EZH2 | design | |
| | | | CATTTG | | | |
| dz825b | ps2046 | F | cttgtggaaaggacgaaacaccgAGATGAAAGCTTCTTCTGAATCC | targeting | random | 561 |
| | | | TTCCGAGTTGTGCAGCTCCTCAGTCACAATCAGGGAA | STAT3 | design | |
| | | | GC | | | |
| dz825b | ps2046 | R | ACgcacactggacgcgcAAAAAAAAACTCGGAAGGATTCAGA | targeting | random | 562 |
| | | | AGAAGCTTTCATCTGCTTCCCTGATTGTGACTGAGGAG | STAT3 | design | |
| | | | CTGCAC | | | |

1-30. (canceled)

31. Cas13 proteins, wherein the HEPN domain of the protein comprise at least one RXXXXXH and/or RXXXXXXH motif, where X is an optional amino acid; preferably, the HEPN domain contains 1-9 RXXXXXH and/or RXXXXXXH motifs; more preferably, the Cas13 protein contains 2, 3, 4, or 5 HEPN domains; in a preferred embodiment, the amino acid X adjacent to R is preferably N, Q, H or D; or
Cas13 proteins, which comprise amino acid sequence shown as any one of SEQ ID NO: 1 to 78, or comprise the protein having at least 70%, 80%, 85%, 90%, or 95% homology with the sequence of any of SEQ ID NO: 1 to 78

32. Cas13 proteins according to claim 31, its RNA cleavage activity is retained.

33. Cas13 proteins according to claim 31, the HEPN domain of the Cas13 proteins has at least one nucleotide mutation.

34. Cas13 proteins according to claim 31, the Cas13 protein is fused with one or more heterologous functional domains, wherein the fusion is performed at the N-terminal, C-terminal or internal of the Cas13 protein;
preferably, the heterologous functional domain has the following activities: deaminase such as cytidine deaminase and deoxyadenosine deaminase, methylase, demethylase, transcriptional activation, transcriptional repression, nuclease, single-stranded RNA cleavage, double-stranded RNA cleavage, single-stranded DNA cleavage, double-stranded DNA cleavage, DNA or RNA ligase, reporter protein, detection protein, localization signal, or any combination thereof.

35. Cas13 proteins according to claim 31, the HEPN domain of the protein is identical to the HEPN domain of any one of the sequences shown in SEQ ID NO: 1 to 78.

36. Cas13 proteins according to claim 31, at least one of the HEPN domains of the said protein contains RXXXXH, RXXXXXH, and/or RXXXXXXH motifs, where X is an optional amino acid,
preferably, the amino acid adjacent to R is N, Q, H or D,
preferably, the HEPN domain contains 1-9 RXXXXXH and/or RXXXXXXH motifs;
more preferably, the Cas13 protein contains 2, 3, 4, or 5 HEPN domains.

37. Cas13 proteins according to claim 31, the HEPN structure of the said cas13 proteins contains the HEPN structure of the protein shown in Table 2.

38. A nucleic acid molecule, which comprises a nucleotide sequence encoding the Cas13 proteins of claim 31;
preferably, the nucleic acid molecule is a codon-optimized nucleic acid for a specific host cell;
more preferably, the host cell is prokaryotic cell or eukaryotic cell, even more preferably is eukaryotic cell, and even more preferably is cell of human source cell.

39. CRISPR-Cas system, which comprises: (1) the Cas13 protein or its derivative or its functional fragment according to claim 31, or a nucleic acid molecule, which comprises a nucleotide sequence encoding the Cas13 proteins of claim 31; (2) gRNA targeting to target nucleic acid;
preferably, the gRNA sequence includes a direct repeat (DR) sequence and a spacer sequence complementary to the target nucleic acid;
more preferably, the DR sequence includes the nucleic acid shown in any one of SEQ ID NO: 79-234, or includes the derived nucleic acid from any one of SEQ ID NO: 79-234;
the sequence of the derived nucleic acid is:
(i) a sequence that has one or more (e.g., 1, 2, 3, 4, 5, 6, 7, 8, 9, or 10) nucleotide addition, deletion, or substitution compared to any of the sequences shown in Table 1;
(ii) a sequence that has at least 20%, 30%, 40%, 50%, 60%, 70%, 80%, 90%, 95% or 97% sequence identity to any one of the sequences shown in Table 1;
(iii) a sequence that hybridize with any of the sequences shown in Table 1, or with any one of those in (i) and (ii) under stringent conditions; or
(iv) the complement of any one of sequence shown (i)-(iii), the condition is the said derived nucleic acid is not any of the sequences shown in Table 1, and encodes an RNA or is an RNA, said RNA substantially maintains the same secondary structure as any RNA encoded by any one of SEQ ID NO: 79-234,
preferably the said spacer sequence has 15-60 nucleotides, preferably has 25-50 nucleotides, more preferably has 30 nucleotides.

40. The CRISPR-Cas system according to claim 39, the target nucleic acid acted upon by the system is target RNA;
preferably, the target RNA is mRNA or ncRNA, including non-coding RNA selected from the group consisting of lncRNA, miRNA, misc_RNA, Mt_rRNA, Mt_tRNA, rRNA, scaRNA, scRNA, snoRNA, snRNA, and sRNA.

41. Carrier, which comprises the nucleic acid molecule of claim 38;
preferably, the carrier is selected from viral vector, lipid nanoparticles (LNP), liposomes, cationic polymers (such as PEI), nanoparticles, exosome liposomes, microvesicles, and gene guns;
more preferably, the vector is selected from viral vector,
more preferably, the viral vector is selected from adeno-associated virus (AAV), adenovirus, lentivirus, retrovirus, herpes simplex virus, and oncolytic virus.

42. A delivery system, which comprises (1) the carrier of claim 41, and (2) a delivery carrier,
preferably, the delivery carrier is nanoparticle, liposome, exosome, microvesicle or gene gun.

43. Cells, which comprise the CRISPR-Cas system according to claim 39,
preferably, the cell is prokaryotic cell or eukaryotic cell, preferably human cell.

44. Methods for degrading or cutting target RNA in target cells or modifying the sequence of target RNA in the target cell, which include using the Cas13 proteins of claim 31.

45. The methods according to claim 44, wherein the target cells are prokaryotic cells or eukaryotic cells, preferably human cells.

46. Methods for screening cas13 proteins, which involves selecting Cas13 proteins which HEPN domain contains at least one RXXXXXH and/or RXXXXXXH motif, X is an optional amino acid; preferably, the HEPN domain contains 1-9 RXXXXXH and/or RXXXXXXH motifs; more preferably, the Cas13 protein contains 2, 3, 4, or 5 HEPN domains.

47. The methods according to claim 46, the HEPN structure of the screened cas13 proteins contain the HEPN structure of the proteins listed in Table 2, or contain the HEPN structure having at least 80%, 85%, 90%, or 95% similarity to the HEPN structures of the proteins listed in Table 2.

48. The methods according to claim 46, include:
1) downloading bacterial genome and/or metagenome sequences and identify CRISPR array region;
2) analyzing proteins located upstream and downstream adjacent to the CRISPR array region, and selecting proteins whose HEPN domain contains at least one RXXXXXH and/or RXXXXXXH motif as candidate Cas13 proteins;
preferably, the HEPN structure further contains at least one RXXXXH motif,
preferably, the amino acid X adjacent to R is preferably N, Q, H or D.

49. The methods according to claim 48, 6 proteins located upstream and downstream of the CRISPR array region adjacent to the CRISPR array region are taken for analysis.


SEQUENCE LISTING (562 sequences)

SEQ ID NO: 1 - protein, 499 aa, synthetic construct
MPAEQRKNWKGYQHSQLQQSLAYFEKRPQEAFLLLKEGWDTSDGSSYWNNWVMNSFSENNRFEKFYENYLMKRVKYFSELAENIKQHTHNTKFLRKFIKQQMPADLFPKRHYILKDLETEKNKVLSKPLVFSRGLFDSNPTFIKGVKVTENPELFAEWYSYGYKTEHTFQHFYGWERDYNELLDNELQKGNSFAKNSIHYSRESQLDLIKLKQDLKIKKIKIQDLFLKRIAEKLFENVFNYTTTLSLDEFYMTQEERAEKERIALAQSQREEGDKSSNIIKDNFIWSKTIAFESQQIYELAIKLKDLGKFNRFLLDHKVLTLLSYDQNKIWNKEQLERELSIGENSYEVIRREKLFKEIQNLELQTLSNWSWDGINHPREFEMEDQKNARHPNFKMYLVNGILRKNTNFYKEGEDFWLESLKENDFKTLPSEILETKSEMVQLLFLVIMIRNQFAHNQLPKVQLYNFIRKNYPEIQNNTAAELYLNLIKLAVQKLKENS

SEQ ID NO: 2 - protein, 483 aa, synthetic construct
MKIWLVTIGSSDVQLRTNDDWGDWYRVIRKDCHNLPFEPAQVIRDSAEPYRIAPRVLGIAYQAYPDEIWNALEFPLLKEFSIKLKDEAIDQIILLTTDQSYIFDENQRDDYKCPYWQDTSELQPILERYFQQHFPAVEVVLKVLAPQKGEFGLDDWNFVLDLVRSTLKELSGEPTAVYISHQAGTPAISSAVQFCSLARFRANVEFVVSNEYCPEQTRMISRSTYLRGLQTQEAKALLERYDYSGVKALLAPYLQAEVANLLEAAIQWNYAKFDRFAEHLQQAADIELAEFAKSRTTLENWWWTAYESAYLGFVRLKQGNTVEAMFHSFRSVEGLLRQWSNVLYGDEIKQTKHPRSEENQRWDRNLNAYGEDLYWFLTVKKSVDVTKNLKQNTTPDIFIFGNLVFKKRNDLFHQLKGLEGQEKVFENWQSPNEPKWKQDPEGHWKLRVLKCLNFICGENFSSLEEASLLVKVHQKIERAISQL

SEQ ID NO: 3 - protein, 447 aa, synthetic construct
MPVWDEDGMEPVLGMSVPVDRDTLIRRWYVHRVRLDSEGTSRRVRIGTEAFARPIEWFALDECEMTERGLSTPAVEQIVRSRSTRPASTWGTGRTVKVPPAQARAQTLLRKLDDARLVGSVVVVTRVRHEIAALTGVEGELEERLAAVVADADRWLQEQERARQELFHDLEQAVAGQEADVVRPLLVRANAIASHHRTEAETVIADAAAACLDAQVRQRQAEVATLRAAQAEVRARGEAERVKELLAALEGRGTGRSRRAMRETVKDLARAAAAAGPYLEAHQRHQVETWRRHAEGARALGQIARQKRSSPPPREARPKRTSPSQEQARTPPLYDKVGRRSWFTEGCPRCQAPKGRPCFNDDDSSGRDRRPLPHEERLELILSTRSARAQQPRRQPPSQMETASPTWRVEDVTCPECDAARGRNCATSNGHPHQRRKERFRLRFPSA

SEQ ID NO: 4 - protein, 398 aa, synthetic construct
MPKIEKWGLLPNTVLGILHKKEWENRRNLKQRLLNQRPFPIEISLKVPTDQQARDNLGHFHAFFKAWTDFAYPELVEWQERQYRQLSEQRVPVKLRIFSLQQLTELLGTEQQLENWLDKISSFLKQSFVHDRHQHRLFQTLIHHLEQIERYSQQEWQWLIQLIEQLKPNMGAGLYLRALPLSAVNTKFLEQNLLLTEAICNVLYDDEIVLAGGLLAWLDCLDSPKGWLTIKPVCPNVQEKLGGLPVFQLSTEVLDQFELPAERIIVVENIQSGLACPYLENSIVVCGGGKNITWMKAKWLQYKQVFYWGDIDSEGLNILSMVRQKIPHVIALMMDEATVLQFQNHMVDEPDSIFSEPQYLTADELKLFHDLRAQTFKNRRLEQERISVDWVHRYLELL

SEQ ID NO: 5 - protein, 382 aa, synthetic construct
MNESSSPLRGVATRGSGGSGRGRQRSSSPLRGVATHAPTGQALPLGSPHRPYEGSQPWLTRVAEWMHNSPHRPYEGSQHARGPVGLPTTKSSSPLRGVATVTHDLRQLRHRGPHRPYEGSQPGRSAVVFAGSASSSPLRGVATGERVPAGCPEHVLIAPTRGRNRINSIARSQFCGVLIAPTRGRNRVTKIEQEIKGSPHRPYEGSQQHEGAVVDGTQESSSPLRGVATRRRRSSSWCRRCVLIAPTRGRNRFTRTRSPGSSAVLIAPTRGRNTRTTRSRFRSVSVLIAPTRGRNTADGRRPTPDLPSSSPLRGVATRRTRTSRTCSRGPHRPYEGSQRAGRDQLRHVPAVLIAPTRGRNTRSRGRAGPSHGVLIAPTRGRN

SEQ ID NO: 6 - protein, 359 aa, synthetic construct
MRGSARPSVRSLPRRQLRNVAGNSANCACSSLPRRQLRKISLIDLDTVQRSLPRRQLRNRSRHKARWAAGSLPRRQLRNRQARRRPTDSRSLPRRQLRNVVSRRPPQPRCSLPRRQLRKPTTGVARTWVGSLPRRQLRKPTSPSRMQRFRSLPRRQLRNIRLHSVQINQCSLPRRQLRKAAPLQTVLSACSLPRRQLRNPRPLEQHVQGGSLPRRQLRNAAGGGTSVSRRSLPRRQLRNVGADGDNTQAGSLPRRQLRNLRHTETEFRDSSLPRRQLRNKQRVKNTFVTCSLPRRQLRNRCPDCRSQDLCSLPRRQLRNLWGNHFRKVHRSLPRRQLRNPRAVGVAPCFGSLPRRQLRN

SEQ ID NO: 7 - protein, 350 aa, synthetic construct
VLADKPSRFLVPVRPALVDAEVVALLYARGQKRAEEETCSGLSASGPTTWFAGTVNPKKAGPSPRKDHHSKSYYRPSTLLALGDRRSSRKTATTWRGYERHTPCGSSPSGPAGCDPGSPRGRPPPGGQRRLVLDAVDLRSSAVPKGDRHVSSTGSGVPRSTSLRSSVVPKGDGHPSSMLRPIGKPAFALKGNGHLTVDIYAIVAFKLRSSLVPRAAATTDHPRIATAARGCDPRSPRRATATFSPSRGVVGALGVAILGRPEGRPPRSSWFCSSPVSSGCDPRSSRRTTATRRPPGPRKARHRGCDPRSFPRATATRQGSRRHRRHQGVAILGRPEGRPPRRRGRSASST

SEQ ID NO: 8 - protein, 320 aa, synthetic construct
MRTGRPHCPPTEVNPCADRTGADRRRGRHRAGHLRLRGENPASGTWSLVSGGLPPPARRERGLRYRPDHPDRTTSACAERTVCGSPAGCPAPDHLRLRGENAAQFAFDLGADGPPPPARREPSATRDEFLNDRTTFACAERTRRRCRAGCGSADHLRLRGENLDTESWATSGYGPPPPARRELGGARGALLGGRTTSACAERTLKRRRDLAQVVQASPETDKRARLFSGDLAEGARLKCGKAIDPRIPEGSGHCSGVDEVTDRRVRGWEPSVLAPQARINTQHLWRALRRHGQLQNGSTVIAHRITCHRQNVRSPSKLLP

SEQ ID NO: 9 - protein, 432 aa, synthetic construct
MIYLQEKVNKTIDVRNYKTGKTSTIDKSWMMTTFYKREWNQEVGKQLTEVKLPDNLSGIPFTLRQLKEKASYSLDQWLNNVTKGKVAGDGKRPINLPTNLFDETLINLLQNDLEAQQVEYPTDAKYNELFKIWWRKRGDSTQSFYNAEREYVIFDEKVNFKLQENAMFTDFYSDSLKKAFRAKQNTRRIEQRSNRRLPDIQFSQVEKVFKRSISNTEKQIRLLKEEDQIMLLMLEELMSSDLDLKLNQIDTLLNKTITVKKPVTGNLSFGDKSEITRTIIDQRKRKDHSMLHKYVYDRRLPELFEYFEENEIPLQDLKNELEAYNTAKQMVLDAVFKFEEDIVTNNQVHDLIGSACDTGHIQHKVYLQWLKKEGMINENEYLFLNRVRNCFSHNLFPQKRTMSLFVNQWADSNFALQIAEHYNEKINAILAI

SEQ ID NO: 10 - protein, 533 aa, synthetic construct
MSGLPLPKPLRRWLLGTLSNQERKAQDWLQSKLEENLRLLLEAKRQLRKLAEPPNRPFLRAGDLATWIARDIAYWQHPDGRQLNSEEYGELQAALAFFGREGAEQVYELFEKLELVGQPNRDHPFLFRLKNRMCPARSRKRQPKPIRGILEFYIAYLRAREKFLTRLQGRLKSGSNPTPEMEHWIPPLRSRSLPPRMNERAVVLPRGLFRRRFVELLPPPDGSPADTPLEQLTLMKLLEPYLDTPQAFYRYERRYARNEHQYTTDGCNLSELIPQIENEATQGSEEEDPDFRRKDLLCLARTINDTERRLRRYQLQDRLLWMIVRQELEREKNMNLQGWTLENVGFDQPDSPLKQEITFQLEYPLEGRNEPIRIEAKLRPRHYGKMRRTLHDRRLPGLLQWLNTESVDWERLQQELTKTITRQDEVLQLIFELEKAVHSRFEKECTAKKNRDYLKHRNYLEVLREHQILRSDEEFEHLLNLRNRFAHNEVPSPAELPLWKDERPGNGNLMEWLIEKSLQLYRDLLERVQGKGD

SEQ ID NO: 11 - protein, 688 aa, synthetic construct
MGFISVHDLRKLLLMELLCEGSFSRMQSGFLRKANRILDETAEGKLQFSALFPEMRHRFIPPQNPKSKDRREKAETTLEKYKQEIKGRKDKLNSQLLSAFDMNQRQLPSRLLDEWMNIRPASHSVKLRTYVKQLNEDCRLRLRKFRKDGDGKARAIPLVGEMATFLSQDIVRMIISEETKKLITSAYYNEMQRSLAQYAGEENRRQFRAIVAELHLLDPSSGHPFLSATMETAHRYTEDFYKCYLEKKREWLAKTFYRPEQDENTKRRISVFFVPDGEARKLLPLLIRRRMKEQNDLQDWIRNKQAHPIDLPSHLFDSKIMELLKVKDGKKKWNEAFKDWWSTKYPDGMQPFYGLRRELNIHGKSVSYIPSDGKKFADCYTHLMEKTVQDKKRELRTAGKPVPPDLAADIKRSFHRAVNEREFMLRLVQEDDRLMLMAINKMMTDREEDILPGLKNIDSILDEENQFSLAVHAKVLEKEGEGGDNSLSLVPATIEIKSKRKDWSKYIRYRYDRRVPGLMSHFPEHKATLDEVKTLLGEYDRCRIKIFDWAFALEGAIMSDRDLKPYLHESSSREGKSGEHSTLVKMLVEKKGCLTPDESQYLILIRNKAAHNQFPCAAEMPLIYRDVSAKVGSIEGSSAKDLPEGSSLVDSLWKKYEMIIRKILPILDHENRFFGKLLNNMSQPINDL

SEQ ID NO: 12 - protein, 420 aa, synthetic construct
MNTVPASENKGQSRTVEDDPQYFGLYLNLARENLIEVESHVRIKFGKKKLNEESLKQSLLCDHLLSVDRWTKVYGHSRRYLPFLHYFDPDSQIEKDHDSKTGVDPDSAQRLIRELYSLLDFLRNDFSHNRLDGTTFEHLEVSPDISSFITGTYSLACGRAQSRFADFFKPDDFVLAKNRKEQLISVADGKECLTVSGLAFFICLFLDREQASGMLSRIRGFKRTDENWARAVHETFCDLCIRHPHDRLESSNTKEALLLDMLNELNRCPRILYDMLPEEERAQFLPALDENSMNNLSENSLNEESRLLWDGSSDWAEALTKRIRHQDRFPYLMLRFIEEMDLLKGIRFRVDLGEIELDSYSKKVGRNGEYDRTITDHALAFGKLSDFQNEEEVSRMISGEASYPVRFSLFAPRYAIYDKR

SEQ ID NO: 13 - protein, 558 aa, synthetic construct
MSPIIWVIICIATLVIGFVLGVIYRKKVAEAQIAGAEEEAKRIINDALKSAESKKREALVEAKEEIQKSRTEFEHEEKSRRAELQKQERRLQQKEETLDRKTDALDRKTESLNAKLANVEAQQNEIRTIRKTQQEMLEKISGYTIEEAKAYLIETLEAEVTHEKAAKLKEIEAQYKDEADRMAREIVATAIQRCAADHASEVTVSVVPLPNDEMKGRIIGREGRNIRAIETLTGCDLIIDDTPEAITVSSFDPVRREVARLALEKLIADGRIHPARIEEMVAKAQKEVNATIKAEGERAVFETNIHGIHPELVRLLGRMKYRTSYGQNVLQHSIEVSHISGLIASELGANVAIAKRAGLLHDLGKSIDHEVEGSHVTIGVNMARKYKENEAVTREHRKLRQASGEARGGHEVLPRHRQLLCHPGRTRDPRDGQARGGQRGSDDAARARHRQEDRRRAHLSRPDQGPHHPRDQGRRLREIKPNVCPRKGNASAGVFLSKGNVHESHHGDRAEGREARHLYRAGLSRRALEADDLSGQQGAGAVRPLVSHAVRHGQRLPV

SEQ ID NO: 14 - protein, 465 aa, synthetic construct
MTDLSRIGAVLIHPRGDHMGNLLWPGVLPADQAAALAASIAAVRAQGYWASPFPEGDGVTFKRYGESSHDAAACLADFEAAFAFLEIEELDAAEGRASALARLAAGRTIMCTYLVPVEALRLERPFLLGDTRFHAPVDGEEVRLANHAWQSLCDVPGADVDPSWVPSARASGTTELLGYPLIERSVEVPLSVFYEAQASFDRQSPLLRLVMEDADHALDPIRYDLCHYRRLEYLPAKPGWIGETAIAYVMPEGRVVKPRLLQGKPYVLRVSNNWLGLEVDPSGFSWAEPLASMVVDDKSGDEVALALKAALRAFNRAFYLVELEASFLHLVYAIDALCEPDKLRGDRHRLWISAFGASGDPGRFATLLGDFDRRYGVRNRIVHEGATFAGLGLKGEEQCQFMLGLLGACIQIFVREGFTTRREAADYAFDFLTSSPVTTHVTAMASEHFKLPLATDKGFREHMQP

SEQ ID NO: 15 - protein, 485 aa, synthetic construct
MLETLAAEYIAASFSVLEAENAIPTSKMHRHLQAHLDFDGSAIEAMPEFAALAQAVIKTGPEGLPASDTNPGPEHTLRLLLFSLLELCVHRCADVDDYDPASTEVSAVITEFCDVVGGSSYNLVVARVVSHLVPATGTEVDIAGITLVAETPTQDLRRCIVAAIPDADHAWKRRGPMLDRPPHSLLVVRDPVEGVGYHGSWNLEGVLDRLQLAVCLLTGANVQSVYQVSGVSSRISARPARVEFLTRDEEVPLVRRVARLDETHVPVLAELSALIDNVRSENGQEIWLSSFGEALTKFARLDDTSSHFDQIVQLSTALEGVILGANEGEGLTLRLCTRATALLAYDDEPAAELFEDLKRLYSFRSTIVHGGEMTTKQLRKDLRAMRCVPDESSGPGLLPRVGFAVDRLRDIVRRAILARLCLSSGDTPLWSIFSNEAPKLDIVLCDDSNRAQWRSHWRDVLASIGAETAGHRAAAPVHSLSDDDM

SEQ ID NO: 16 - protein, 164 aa, synthetic construct
MPIKENNLIFPYPPSINNPNEESINYKKFYTEMGKSVQKYNLTNTFNAAVRMKVILHAKDNTKRDVDNPVKALLDAFTYAGVWVDDNQVRDLRIIYGKSDSKGEGYIEVSMNEATDLSNLICDEVKQKIKIKNQKIKNKPTPSPAAQQNQKKRKTRLVINNNHQ

SEQ ID NO: 17 - protein, 177 aa, synthetic construct
MKMFYIEHKTPYLYVDCETTGLKPGTDEVLSISVIDQTGKIAFNSLIRPERRKKWVEAQNINHISPEMVRDAPTYRDVVPQLQRIFRGAHIVTYNLEFDYKFLHEAMCTASSFHCCMKAYAKFQKEIDPYRGTYRWHKLTAAYDNLFHNTFNAHDSLADVQATLRVWNRIMHDYKVR

SEQ ID NO: 18 - protein, 199 aa, synthetic construct
MKIAYLDIETNYTGKFTDQRLFSDHENHLITVLGMRLFQEDKDSFLQLVRKDVSKGALLSALRGTDLIVTYNGRSIPDKMKGYTGFDFPVIAAQLGVVLDEEFKHVDLCPVCWQRGLWGGQKTIERSLGLKRKLPGRDGAWADVTWKKYETTGYYNFLEELLEYNREDVFMLRRIQEALRCTENYQRPRRSCSHAAPLL

SEQ ID NO: 19 - protein, 252 aa, synthetic construct
MTVRIMQSLRLVVACVLAFLLTNQGTAIADNCPPKGCAQKYTLALSWQPGFCETHGYKAECQEQTATSYDANNFTLHGLWPDKLEYCNVSASYIENDKTGNWKDLPIVEVDTETTDELDEVMPGFGESSLERHEWIKHGTCDGRNSDNYYDLSVDLLKEFNDSQVRNLFIEHIGETVSLDQVKTAFEGTFGSGSSSSLNLKCDSTNNLATEIRLKIKRPQSGQKLADLLLPSGGQSCNQVVVDGFGVSNLTQ

SEQ ID NO: 20 - protein, 302 aa, synthetic construct
MHAVIHTRTVSGLADDAHTVAALAWSAADGEHRSQLFIRPGASCEARYAAGLDAFEMALALCETNDDTVMLHINDHGLRANIDMVLDAFPHIHLAHGTRGNLLALLETAMHRIDEHTHVAAAVHHERLERARIERAQALAELPELVIATDASKATHRRGAGIAYATADGRYRQEFLSGVRHISVGELLAIEAAVADFPGRRLHILTDSRDAMQAIGVEWEPRWRVTGETATLGARIRALMSGRDVRISWVKGHSGHELNEIADRLAVAARRHHESKVPAASRRSVADNIVAPLRAAHVQAAA

SEQ ID NO: 21 - protein, 267 aa, synthetic construct
MDITMLGTGNALATECYNTCFVIDDGGRLFMVDGGGGNGVLRQLRAAGFDWMGVREIFVTHKHIDHLLGIVWMMRVICQSMDQGVYEGEAHIYAHDEVISLLRWLADELLVPKQTRFIGDRLQLIPVVDGEQRIINGRRVTFFDIHSTKAKQFGFCMELDGGEKLTCCGDEPCSPYGEQYANNSAWLLHEAFCLHAQADVFRPYEKHHSTVKDACELAERLHVRNLLLYHTEDSNLAERKARYAAEGERYFSGKLWVPDDLETITIQ

SEQ ID NO: 22 - protein, 464 aa, synthetic construct
MDYPQRKAKPQQHFKGKSSKPNRFSPGTRTGRRPSSEYDFFTGGGTKITNTDKESTKTADFTIFYNIAFENVEKVKTQLQNKTQSQQNAFWAEYFWKAHFLSKNTNGYELTQQDDRIITQLIKKVEEIRNYHSHIWHDNSVLVFSDELKRFVEQKYNEALVQLSVDFPGAVSDYQFLKQKNYEKESKLFNPIGFGGDAKNFITIEGRIFFLSFFLTTGQMNQFLQQRTGYKRADMPQFKIKRLLYTFYCNRDGAAITDFNHEDRFIDTLAPEVRQNVFKARTAFKLISYLMDYPDYWGSNDAMPLFDNNNELIKNVEQLKDYIESKNILPELKFTLIDRKIKASLELEEDAETLKKEQEDKHSTGTIAFTYDELSGFSFHINFEALHRLVLLQTLHHNMVDLPAPLAILAIELKKQANNRTTLYDILIKPIAERTDDEQVYLLTKENQYLRGGRKVTELGIIFF

SEQ ID NO: 23 - protein, 509 aa, synthetic construct
MPKWTYSITIVNNMDRPLELISSSIAWGKKAKAFPTEIAAGEHGEFSVYAKAGTSTGIEFYFTMGDKVEKKGDPHYGNFSFSVDMPYWKHANKSSFDCTGILTQSGFKKIPDGAHDFAITATVSTSLCSIFLPGEKTWKGNGYQMEYCNLYDWEEIQKLKVVEPDEKRIDDFIPVENIFSKRKTDSRTEQVSVPKKLWKEIIDRKYPDDYSKRNFVKDYFTVSAYEIRKNVTVSIAANQSYEKTLEVSNRSTVRRETREEFQIENTISGEGTGEKFTLSETLRMQYQISRLDEYCEENMESVREVFNYDAIEQDRDVVLWDLAEVLMLYRVNIKGKVELVGIGDYYVASTQKTYLRNGIQGEESPYDTREEELPCADSESNYRLYDDSDEELLGEEFESWDDGDGVPVKSLGVVQGNIRINNVNYRWMEVRRGTNRGMLFNPGEHRYSFRPNPHDDPWYNRHQTDWYEAMAREFQRVGNANHWTQDNWSDGQDIRNLRVNNETYTARVY

SEQ ID NO: 24 - protein, 413 aa, synthetic construct
LSSLISKFQQIEAFNGDIRNLKLRNPELNLDIMDDGKIGYNSKKYSIFENSGQIANDLDKLRGVIKMDINDLDAYEPMIKDALRVIGVDGSDLDSIYKDYFNKKDKKNSVAGFFRNNIINSRRFRYIIKYINPSDAYRIIQNEHVRNYVLGRMNDAIIDRYAHSVGIEDKVHDKRKVLSDILSKVKFDNFTKLTYINPKDKNKGEKAKEREKPKAILGLYLTIVYLVVKSLVRINSQYVMAVYHLERDSRLCPGVSSNNPLSMTSMTKHYCDRRNNHLLKEKHIVKLERYQNTPQKICTAYRNAIAHLSAVRKGVKYIGDIQKADSYFGIYHYCMQKFLYSADSATRHPFAEFVRSIFGDEKELDKLRNGSYSQAILRALNYPFGYNPARYKNLSYEKIFMRAWQDEDTNKKT

SEQ ID NO: 25 - protein, 581 aa, synthetic construct
MNYRLGLDLGTNSIGWAVVRLDENGDACGILDMGVRIFPDGRENSQSLDPLGQSLAVNRRIPRGQRRRRDRYLKRRSDLLDKLTACELMPQDLETRAAIAALDPYPIRKRALEEPLSPYDLGRALFHLNQRRGFKSNRKAQPKDDEKQSRENLSALDIALKESGQTLGEFLAPRHEQSEPVRFRPGVGLYPDRAMYEAEFEAIRKAQELHHHLHPGQWDSLRDIIFFQRPLKPVDPGWCLLEDGERRAARAMPSAQEFRMTQEVNNLRIRVDIEPERSLNDAERERVLQRLREGKDIDLQKPTKSLQLEGSFNLARGGRKKLQGDQTTARLAKPELFGKRWFDLPLEERDAVAARLLDTEDPKTIRQVAVNEWRLNKEQAARVADVTLPDGHTHLSLKAIRNILPHLENGMRYDEAVQARYPHHSDFRNAEAHDRLPYYGVVLERDVVGADPNAPDGDEPRRYGRFPNPTVHLGLNQLRRVVNRLIDVYGKPAETVVELARDLKSNKEQRERYVRQQRSNEHENDRRREELRECGVEPNGYVMEKLRLCNYKPHRALIVISLSDGRSGRNRNPSCCSSRSP

SEQ ID NO: 26 - protein, 523 aa, synthetic construct
MSSRGGGQKSAVCARALQRPNFFTYTPNRRCARHAVQPIGALTAARYGCKMRPYRKGTMSEQEAAQEGTKLLAKTLTFGLGNPMGFKSKGSVLVELTEDQRKAIYNGLRDASTVVARIINLLNSREYIRQIMKVPEELVAQFKPNYSLVKGPLKRLGIEEAEQVAGSVLSQTFALGVKPDFQGEHGKGLLLKGERQIPLHRTDGTHPIPQRATETRLFQVEKNFYVAMQVFAETWAKKQELPSGWLAFPIKVKPRDKTMAGQLLKTIGGEWKLKNSRLMRNPRTGGNRWLGQIVVAFAPEPFKKMTRSVVMGIDLGVNVPACLHISENGKPLPWAMMVGRGRDMLNTRNLIRSEIVHIIKALKSKDSPLDGKARAIYRDKLRDLRKRERRVMKMASQTVAARIADTAKRHGAGTWQMEDLSPDIKTDQPWLARNWAPGMLLDAVRWQARQCGAELVMVNPAYTSQRCARCGHIDPQNRPKQTDFKCMACGHEDNADKNAARNLSVVGIEKLIADFKAPNGAVQ

SEQ ID NO: 27 - protein, 191 aa, synthetic construct
MMLYNWIEGSITEAIDEIFEAITQQKVRFDELIPAYQRLWLSYQHGLVKIAAESAKAKDPTKNKIGKSLPNVLQQVQYFKILTFTDKEGIQYKNYRGYLKVVDTADLSGNLDARKMRELASKYAFSVPERCDDLLKIKNIRNQLAHGEIIFSEAGTEPISRLMEIKGNVLEYLEAILLNINEFIEKDGFKI

SEQ ID NO: 28 - protein, 789 aa, synthetic construct
MENIKLEKQKAAFYFNQAELNLKAIEGNIFDKGRRKTLFDNPKILSKVENFIFNFKDVTKNAKGEIDCLLSKLMELRNFYSHYVHKPDVKELSKGEKPLLERYYQIAIEATGSENVKLEIIENDKWLTDAGVLLFLCMFLKKSQANKLISGISGFKRNDTFGQPRRNLFNYFSVRERYKVVPDMQKHFLLFVLVNHLSEQDDYIEKAQQPYNIGEGLFFHRIASTFLNVSGILRNMEFYTYQSKRLKEQRGELKREKDIFTWEEPFQGNSYFEINGHKGVIGEDELKELCYALLSYNKSKYAVEQIEKFLKGFGEVKSEQEIRDSDILNESYFPTNYFAESNIGSIKEKILNRLGKTDDSYKKTGTKIKPYDMMKEVMEFINNSLPADEKLKRKDYRRYLKMVRIWDSEKDNIKREFESKEWSKYFSSNFWMAKNLERVYGLAREKNAELFNKLKAVVEKMDEREFEKYRQINSAEDLASLRRLANDYGVKWEEKDWQEYSGQIKKQISDRQKLTIMKQRITAELKKKHGIENLNLRITIDSNKSRKAVLNRIAVPRGFVKEHILGWQGSEKVSKKTREAKCKILLSKEYEELSKQFFQTRNYDKMTQVNSLYEKNKLIAFMAVYLMGQLNIRFDKPTRLNELEKAEVDFKISDKVTAKIPFSQYPSLVYAMSSKYADSVGSYKFENDEKNKPFLGKIDIIEKQRMEFIKEVLGFEEYLFEKKIIDKSKFADTATHISFREICDELIQKGWDENKLTNLKDARNAALHGEIPAETSFREAKPLINGLKK

SEQ ID NO: 29 - protein, 643 aa, synthetic construct
MKNRKELLINHFSDIKSACTSLAQVCQIYMTEYNQQNNQIKKVRSSNDSIFDQPIYQHYKVLLKKAIANAFAAYLKNNKDLFGFIGKPFKANEIREIDKEQFLPDWTSRKYESLCIEVSGSQELQKWYIVGKFLNARSLNLMAGSMRSYIQYVTDIKRRAASIGNELHVSVQDVEKVEKWVQVIEVCSLLAFRTSNQFEDYFNDKDDYARYLKSYVDFSNADMPSEYSALVDFSNEEQSDLYVDPKNPKVNRNIVHSKLFAADHILRDIVEPVSKDNIEEFYDQKAEIVYCKIKGKDITAEEQKAVLKYQKLKNRVELRDIVEYGEIINELLGQLINWSFMRERDLLYFQLGFHYNCLRNNSEKKEAYQKIEVNGDSIKDAILYQIIGMYVNGVAVYAPDEKGDILKEQCTKGGAGGKISAFNRYSEYLGLDKLTLYNAGLEIFEVVAEHDDIINLRNGIDHFKYYLGDYRSMLSIYSEVFDRFFTYDIKYQKNVLNLLQNILLRHNVIVEPVLESGFKTIGKQTTSGAKLSIRSIKSDTFQYKVKGGSLITDAKDERYLKTIRKILYYVENEEDNLKKSVVVTNADKYEKNKESDDQNKQKEKKNKDNKGKKNEETKSDAGKNNNERLSYNPFANLNFKLPN

SEQ ID NO: 30 - protein, 410 aa, synthetic construct
LFDQITDWRDIGAHAEDIHKKKLTAKFVEDLRKIYDASWHKTKEGLSLSVSQMDQLVRLKGRQTKNHDKLQVQKDRPNFKYGLINDSNHFSEQGLAFLICLFLQESEANLFLKHVEHVWSNQDFNSNSTNLNSSQAIKLFTAYRLRLPFPKLQSSHDSQSLFLDMINSLKKCPSILFNHISNLDKKKFKKDIKNSEVNFGSEELITNENIETVHEVRKRDRFVIQALEYFDLTNTFNYMRFHIDLGKFIYKTYTSPIDGTDRIRRLEKNLHAFDKIQEVIGEKRPEIWKNLIKEPNTIPEDSLDCYIVDTEPHYHISDHKIGFKVFQNVLPSFHDIVKIGENNFKFSENPKPDGFISIYELPAMVFYEHITGNHGKSEEIIREHIKQRRKIITEVLNDSNLPLYKTSIHL

SEQ ID NO: 31 - protein, 423 aa, synthetic construct
VEEVFNLLRRKSNPPQSKSQLDCDIHEYVEKHRKDKSKEWAEDPTALMRKQAKQVEQTEHAIRRCQIEDIVMLYAARDMLYAARDILSAKNNRTPNGTDTPQPQKFKLKHVQKDDGLLERTIDFDWVVDIDGQQKTIRQQNMKMKDYGKFYKFASDGERLKSLLAHLGGNEFQRADIEAEYANYDVSRSQVFRYVYMLESKAYQLLAKRKDNPIDLLNDQKPIPDAFWFVSKEGIRNEFRVFKENLFNEYKADVHDYKANDPDAEALVNAICNFTGTSIEEWDANQDTLMKQVKSLLTDENPNEKDKALILQVKDYCMKKDIARKAIRNNFGELIEILLRGDEPIFTDDDKYIIQHIRNAFGHNHYLKKEDEYNTVFRGKEAKLKLPEVAKTIKDWMGEKTTKALSLTPDTQRALPPKSQAAE

SEQ ID NO: 32 - protein, 619 aa, synthetic construct
MRIIKPYGRSHVDAADLTERKRVLRLNDGAKTSHDIKSFALGHDELIIAQWISSIDKIATKPSGNKGATPIQRKFRDQLGKTSWALLVEKDLLPGLKDPDKRAQLEKLWEFKVAPYGKAEFRDKLKAPNPKGRWFKRFAGDVDVTTVDADEVARRIHEHLYMAEYRLEGGNKRVGRIEARAKSIERNVLRPAKTSSVADNGWSDADWNTYIGAGDVAKQIWLAARARQEGRDGAGTRRVTASVAAIALAEQYAKLFAGIDGKPLGIKQAKSAAPGLFNLHMAIKDAYSRILKHRKKDQRQSGKPQRKVSNILPPDMCRLIILVKNKTKNQRLNALVREGKVIHYEASQAGGKDSPSVLTDNWPRDTSNSFYWTSDGQAEIKRNEAFVRIWRHVLALAARTLKDWADPNDKIKIDILDRGSIESAIGDSFVIIQHNQKLALLFGKRASVFNDAGDEDVQREVLELALKGIASLRHSAFHFKGLDSFREALTKSDWTSSGAARGALEALWQTDVKARALQRKKTLSGAHVEYFLDESQARKLMAALEQAAPASIPLPRFARVLLRTSHMETKQHDGLHLPMPANRADLEDPARLCQYTVLKLLYEQPFRKWLETRSTQTLN

SEQ ID NO: 33 - protein, 550 aa, synthetic construct
VRERDGERRRVSIRARLGSRAIPSLQGDFGAILEVSIRARLGSRAIHIGQHADCEREHVSIRARLGSRAILDVDEESGIQLEVSIRARLGSRAIHYLAKLGESGKVFQSAPGWEAGRFNCSAMTPAQLLEFQSAPGWEAGRFQVAAALSISQSVFQSAPGWEAGRFPVTAAGVITLRLFQSAPGWEAGRFDYLAKLGEAGKVFQSAPGWEAGRFGRAGHGNRRCPMFQSAPGWEAGRFGASCTMSPSRPMFQSAPGWEAGRFLSRLAPARTSTWFQSAPGWEAGRFMSRPPTNRPKMMFQSAPGWEAGRFGHALGYRDGREEFQSAPGWEAGRFALTGKANTHANLFQSAPGWEAGRFRAAWGGAATDTVVSIRARLGSRAIHRNAKYGATAMLFQSAPGWEAGRFRKIPPPAVGRSCFNPRPAGKPGDSAHPRPSREGAGVSIRARLGSRAILHAAEAEQEPAECFNPRPAGKPGDSRAIAEGHDIIHVSIRARLGSRAILVSTRDWKRSPKFQSAPGWEAGRFGRPRVRAGCGCSFNPRPAGKPGDSR

SEQ ID NO: 34 - protein, 129 aa, synthetic construct
MAFDLPTIDPSLDIEKITTEELRRKGIVLIQKAFWTRGYYRKVSERECRLYFFLCAFMRIEDFTVHLEWGAMEDYLESNRRGVRVALRRLIRKRLISIISHRLADDRDPQKDALTIRIWRLRYGKKEKK

SEQ ID NO: 35 - protein, 123 aa, synthetic construct
MAIILQAPLVSCWHQRGAFGNPIFGRDFHPAHLGTSRHKLSMQECSVRSSLRSCILRIEWELWGKNSHRNGSPVIYGVFIRFWPTLYICAFPAKNTVYKLYIYGSCQPYIYTEYIRYCWQGSH

SEQ ID NO: 36 - protein, 158 aa, synthetic construct
MARLRKLKRKFSTSFRAVLFRMVPKLGKMAEGLTQRFRAVLFRMVPKLLSFQLIECICFRAVLFRMVPKHLEAVKKAQYGFRAVLFRMVPKHGTFNRLSASCFRAVLFRMVPKHGQKSKKTKSCFRAVLFRMVPKQLNKRIVFLACFRAVLFRMVPKP

SEQ ID NO: 37 - protein, 187 aa, synthetic construct
MARLRKLKRKFSTSFRAVLFRMVPKLGKMAEGLTQRFRAVLFRMVPKLLSFQLIECICFRAVLFRMVPKHLEAVKKAQYGFRAVLFRMVPKHGTFNRLSASCFRAVLFRMVPKRNPSFPSVPSCFRAVLFRMVPKLRILDTLLECCFRAVLFRMVPKQDLKSIRIDFSFRAVLFRMVPKPSLETEQN

SEQ ID NO: 38 - protein, 289 aa, synthetic construct
MAYFLFIDESGQDHRDSPYEVLAGVAVKDTALWSLILAIQEAEIRHFGMRYSQGNRELKGKKILKRKVFTHAALLPEIQESDRMPLAKSCLENGAGAGKREMVALAQAKLAYVQEVFEICSKHECRLFASIVADREHCDHHADYLRKDYSYLFERFYYFLEDVDPAAAGVVVFDELEKSKSHILLGQMDSYFKLTAKGRKRAKQIIPEPFFVHSDLTTGIQVADLAAYIISWGFRTARLLEPKREELKSLVHIISQLRHHSERHVEGQTNFKVWSFGIIQDLRGGSARC

SEQ ID NO: 39 - protein, 205 aa, synthetic construct
MEGDIYRMLRIQNKIISIHSLRMEGDIYRMLRIQNKIISIHSLRMEGDPDTTTEVQTTEYFNPLPPHGGRQTNHYSETLTSPFQSTPSAWRETSSDWMSRRRPDISIHSLRMEGDVVSVAVSGKPLYFNPLPPHGGRQKTSRHPARESHFNPLPPHGGRRQVLAAVCLFQDISIHSLRMEGDIVMRVGFGYSAFISIHSLRMEGD

SEQ ID NO: 40 - protein, 269 aa, synthetic construct
MEHYFPVKSSKRRNSTAEIKYYLQTDTIKCCLHHQIINAVRGLALREGKFNLHGFDDKLIPNERNVSSSILNELKTSEGFVLNMLGGCAFAANCLRNIVDATQRSDLLGFRCFEVSLKKGKSNSDLFALFFGFGREDMDDDSEWEKHLYAARYSVSEIRNRVAHYHKSAIENIYNITDFKYRENSMCSYTDTKFTTALQNEIYNTPKALSLQLMTGKVLEYYPKEKLVSFFQKYKFSLYRSVVPFAPGFKNIMRTGVNYQNATQNSLFL

SEQ ID NO: 41 - protein, 126 aa, synthetic construct
MGNDYNNYQNKRQGIKPKQPNKVRRIQSRDFRISLTGKHVFGAYFNMARTNFVKTINYVLPIAGVRGNYSENQINKMLQALFLIHSNRSAELTQDSNNGKGSSRSLPNNRHGYNACSSSISPYLVQ

SEQ ID NO: 42 - protein, 197 aa, synthetic construct
MGRLSGRRQPRQRDADPALARLNPSRPMAGYCWRPAHSWQCRREINATELARPTALPARQRAACAAANETIRGQVFNNEFLSCLRGSELHATAFPQGGGFLSCLRGSEHQGQQREGEAVFLSCLRGSERGIPRLERCRKFLSCLRGSEPDDGGISPIPEFLSCLRGSERHQRSRWACRMFLSCLRGSEHESYGCRGS

SEQ ID NO: 43 - protein, 144 aa, synthetic construct
MGYDRKLKNAVAQSVIELVAREGLHLDWGMQVLDGAHVLVDARVRGKQAAHLGNLGLFEKGQGEREKKPAHRLTETLSGPHLAEMAATLFAGRVAHATDAAALPRAAIDWQRSAAPETNTKQAHNHPQRPPKPRIQRPPLQAAK

SEQ ID NO: 44 - protein, 181 aa, synthetic construct
MIPGIAYCRDFNPRSPNGERQPAILSFHGSFQISIHAPRMGSDLPVSVIVSPAGISIHAPRMGSDESCRARVFWHGDFNPRSPNGERLGSCPGLPISRFQSTLPEWGATRHPDHHPRGGAFQSTLPEWGATAKLWQWGGADEYFNPRSPNGERRRRRPQIVADQAISIHAPRMGSDFSSRL

SEQ ID NO: 45 - protein, 257 aa, synthetic construct
MKIPQIIEDNKHLFGTYSTMALANIRNILYHIATLACIENDFNADSDDFWHHPCMEIINPQNLCNDVTKADFVTEKLKSHFPFVVIMAEAKRQKDIAWAKNQAKKAFENRDFQKQQEFNNKQKSFLSITNADIYRMLNNLLRVLTSYRHYTAHYLINYIYFNEESNLLKYNEQPLSYNLNDYFTIALRDTAQKYSYSPEALSFIQSSRYKIENRRKILDTDFFLSIQHRNGDSNPKNLHISGVGVALLICLFLEKNM

SEQ ID NO: 46 - protein, 289 aa, synthetic construct
MKKIDDLTGEIYAKIATRIRERKSQRHKKRNEITDDNSVQLLSNIMNNKRLSSRNPYLLNSKMTYDIVTNLDFKSSYELIWGNGKDLDEMLRIVFEYSLEYLQNKSNDYGKIIEDCLLNFYPYARLSAEYDHAIEPFKPEIPDIGLAYDFAKKHLYFEISDDFKSKHRKYFETLETKKLPDKIITFVEKDVFEILKEYLKKHAEGITTYELISKIIGYETEDMYEDMIHGPEWSAHQPLTYTGETYKKVRQETIDAGRSYIDAIIHEQEETDYFYQIYPYPLTNGDFDY

SEQ ID NO: 47 - protein, 292 aa, synthetic construct
MKVWYMPSWNGDLRLEPLSSDPKRTALSIIDPTDAEKIALGAIGATLLDKGWLKEPILDLLSGESYRESQKVEVVLDGPLEEVGPVVIAALKPGPAVLTAVILRGGKVEIVEHRDPGKAGIKETSKELAAVAKQPAEAAATVKRPTPCCPDCYVDAIKPATQTLLAFLSPEQHETWSRERYIVCRGGLSGHRYILAHRSSPMAARNTRICFDADDRSILHFHDQTVPPEEEVLSAMLILQHREDWLRNEATCLGGGLRRVFKNPFGDIMDGVADSNLTRTIGLLAKSLTPGP

SEQ ID NO: 48 - protein, 200 aa, synthetic construct
MLFQLFEDDKMIEKIRRKLPYMFFIAEIEMSKSGKVGMEVGTLREQILIALLIYKFGYENVKLNPIANEDFDLELYGFSGSGIKLVWTVDWEKVENFYRNYEPRSEIILVEVSWYKNGGLYYIPLEVQREIFEEIGRDRYLYRHKKGTNPRGVEISNEGIKMLIEHPQAKKIDIFWEKPEIGADIIYKPYQRWVELWAEE

SEQ ID NO: 49 - protein, 252 aa, synthetic construct
MLPRIVFFYDMGLTAFQDKDRYIDDVEAWNARYFFMKLIYNNLFLPPRFTNDKNDLFRKTVEQVLEVNRSHMEKVGKDDRQAFGEVRQMRENETANDYMAYLQSSLRMEKNKKELEGNGAKRINFEKFILQMFVKGFDSFLKDGAFEFVHSPENCLTGSTEKERAKERNSLCEEIGNYVRLVSTVIDPGKGSHVAFYVFCKMLNSVYLSNIRNEMIKYHKVVRTDELAHLIEIIELCLLSADAVSQKNSECR

SEQ ID NO: 50 - protein, 203 aa, synthetic construct
MLPVFGWMIGAAVVGAGKFLLEILEDEASSARSEWRERSEELAQKTDEYNRTLERQLTTSAQSIEFKRLTNLYRESRRKADLVHRSLQEGRTLLSRLDCAVRQSAMDKTALIQATQQRNHKARQAARSQLEALNFLRSGLFAERTRIRESQENLLGRVRELNQNTHQLKLQMRDNCGHAGRCWFDKIEGHRLAPPGEINKRKG

SEQ ID NO: 51 - protein, 247 aa, synthetic construct
MNHSEVIVARADKLGDLGEGQTIGEFFFELSAATASEMRIQRAYEPDREKANEQAGYIEKLKWDVVAQAFETYLKEKGFDFVLQLGSGTPLPEHAQFSFNDLPAAEMNLPAEDWQISLYFLVHLVPVADIGKLLHQIRKWNILAGKGGLTESERADDTAAKLNTVFGLYLDMHDAKFEGGAALTGCDRFSSIFESERAFNTVFPAHGSGDDRTPKRGLREIMRFGHFSSLRDIFEQNHVTENDINAL

SEQ ID NO: 52 - protein, 221 aa, synthetic construct
MPLKRTSEFQSTRLSRASTCRLLPYRNLPDISIHKALTSLDRILDDRKKILGNFNPQGSHEPRPMSGRNIPAINNFNPQGSHEPRLFNALPRLIKWHFNPQGSHEPRQQKYTIILASMPNLYILIIHLQQIIQQVKKFSRILILFSGRLQCESPWNFMCNYYSHQSFLTKNQPSFKLPPLSICAKFSRTILNNHLKWRSQLTVYPAVYYVLSRSKNSNVFT

SEQ ID NO: 53 - protein, 116 aa, synthetic construct
MRDSGSARIEFQSTPSGGKATLFSSVTTPYRNSFNPRLPGGRRREHPLDRDARAVVSIHAFRGEGDLLHDATKNLSEVSIHAFRGEGDEEIGWFEDVIDGFNPRLPGGRRPQRAKS

SEQ ID NO: 54 - protein, 273 aa, synthetic construct
MRGKPCSLIPTMSAARIIPAHAGQTRRREKPSCTMPDHPRTCGANAVARACAVLPVGSSPHMRGKLPQRRFFAGRLRIIPAHAGQTFCVATCIIKISDHPRTCGANTPPSRSMPCAFGSSPHMRGKPPACFRQVWLLRIIPAHAGQTRVPYATGPLRGDHPRTCGANAVSCCGYQGMGGSSPHMRGKHVLSYDDHSLLRIIPAHAGQTRRRAGVPRDRTDHPRTCGANDAAAVREFDSLGSSPHMRGKLFAALYLRCLRRIIPAHAGQTCRRR

SEQ ID NO: 55 - protein, 295 aa, synthetic construct
MRSTGKHPHKNNMNDKPNFPGWGVNRVKVDLESSEETLCFRAGVTLDGKLVGHAFNEGGGGPTHVTLTKAALKNIAKPTGIADHVDRLVDNVVALKELAKTHKRWCKEIGVGCVYALQHDELKSWEAGMDHQESTMPPFRAYSSHEAAAKALAGVEHFSYAKGSSKALWEIMVQCHDKWTALEDARTDKMRAEYEAKLQAQLAKAQEGKTSGPEENEDYFRKKPSAEEAHRVEIGKILQRHLAANLPLPTTVRLEALKLLSEACVIIDDEDLSPAALLAYYVQYVVPELLLMPND

SEQ ID NO: 56 - protein, 184 aa, synthetic construct
MRVAKGSRFNSATAVMPWKRLSFRCGCSAGSGFNSATAVMPWKRCSHHDRRRSSSQLQFGHGCDAVETTAWGFGSARPTSFNSATAVMPWKRCRVGRGPRVQRRFNSATAVMPWKRRRITPHTHPRIGLQFGHGCDAVETGRTGTEIQRQQEVLQFGHGCDAVETLTCSSISSLNRSASIRPRL

SEQ ID NO: 57 - protein, 248 aa, synthetic construct
MSRPTIPIDLYRDALEVFHWATKRHFQLWFTGTSQNRHRRTESVLCRLAKNGEARAVRYGSGLIYSLRRKKSEEFRELEKVAHGLACTEGLVRAYRSSLDGEVIAEKFFAGPGSVPEWGIRYPDGKMLLFEFGTKHNFYFSGMMRGKLSAYEANLEKIEEKFQAKAIVLFVLDVPRPAVERYVGSLKRDAGSVADGVPSASSGGDRFPSDPFFFTDYKTFLEVPIGGQLKAPIYIWSDGESYPLRNNV

SEQ ID NO: 58 - protein, 178 aa, synthetic construct
MSVTVYNSLRNQPGSAKRLGSQLPELTAARLIVESQLVNAQQLQESVQSIEANAGWAMWRDALVLCTTLDSARQDVIEGQWCNEQVSLSARLVAPQQYRLVRMHRETDAGADQVYRDQRVLLRDGLDEQHAIYRLWYQKQNTPLLSETAEPTEQWAWRPVAQQFMGWHTSQALQKEIS

SEQ ID NO: 59 - protein, 266 aa, synthetic construct
MTEQNERPYNGTYYTLEDKHFWAAFLNLARHNAYITLAHIDRQLAYSKADITNDEDILFFKGQWKNLDNDLERKARLRSLILKHFSFLEGAAYGKKLFESQSSGNKSSKKKELTKKEKEELQANALSLDNLKSILFDFLQKLKDFRNYYSHYRHPESSELPMFDGNMLQRLYNVFDVSVQRVKEITSTTTRSIPTATSTTLSGKARKIGAAITTIPSSSTILSIERGRSRKLDCCFSSPSFLKKGMPSGCKRRSEASRAAPKPTSR

SEQ ID NO: 60 - protein, 150 aa, synthetic construct
MVATQRKVSTHAPAKARPSYEFAGHALTGFNSRAREGATHSDAQDHVLRGFNSRAREGATQMIPVIYDVDSFNSRAREGATCRRNRRRHSLCFNSRAREGATSYAPHQPKFLQFQLTRPRRRDYLKFAVMSASSVSTHAPAKARQASSKY

SEQ ID NO: 61 - protein, 191 aa, synthetic construct
MVFTYILGVIGIIVTLKVIQMTFIGLQMLKTAVCDPSTNDDGLAVESFTDLLERARSNMTVYDDGNDMKGSIYMQQEVIDAVKEKLENNKRFSLRCYFNCNEPTLFRKEFDSYSKVDIKTRPSGDTRPDDTHYKIIDNGRMAYLSRHEHGSKKREFQVVDCTRVNRFALEGVTDELLGEYKVDIKQKFPSA

SEQ ID NO: 62 - protein, 75 aa, synthetic construct
MGDHKIISHERDKPIYRRLDSHPFRLQNIIGYPRQRFDLLWNRRLLPHKDMEPIGDFAVDDFQRCNLYNCVFLNV

SEQ ID NO: 63 - protein, 72 aa, synthetic construct
MPLRAAAGGVDFSAADAPGAERAAVLRPHGRPETGAEEAPTDQAVPGLRSRLFRAHRGAVKRECLADVTVVS

SEQ ID NO: 64 - protein, 87 aa, synthetic construct
MVAYIDEFKDRFRVGPIRRVLAASLDCGFITPRGYRMFKSRPVSRMAARHEALARDILEIHADSFMAVYGYRKTRARSLARGRDPAG

SEQ ID NO: 65 - protein, 84 aa, synthetic construct
MTEFERLMRASDFCHSMSAIALERNCIDAFHAWRSHARAYSSAARVLHRAQWKRSVTDDGMEPCKRNADGLCRSRRTAFRWGKS

SEQ ID NO: 66 - protein, 61 aa, synthetic construct
MSRLSAIAHLRAYGDNISGLSPQLLVLSLIGVITRNHEMNHAIAVFSSLVSWHIYSSRHID

SEQ ID NO: 67 - protein, 97 aa, synthetic construct
MLVPLSVCMLISPGSQVSSSPAFVTCRTPCITRHPALRALHQRPAPTPCIIRRPALRALHHPYMPQRLPGDHKIMRWYAPCSCLAQWRVARPSSCIF

SEQ ID NO: 68 - protein, 92 aa, synthetic construct
MPCSKRLLSDSQYSQRRHHPGYDRESARPKTCNHPIPRRRRPQKFKKFSKTNPFGRNLSNHNAIHLFMDNTSPRCHPGAALYNCFHANGIPA

SEQ ID NO: 69 - protein, 74 aa, synthetic construct
MLAFFNTRTSLADWDLYSDRNYRLHWKPKHRSLADWDLYSDRNDSIFNLKEGISLADWDLYSDRNCMGQHDGRR

SEQ ID NO: 70 - protein, 90 aa, synthetic construct
MRYYTKHDKSIFGLLGEIYNGFMIYDTKPKKSVSFITQNILERYRVVANIHMEHADDKNFDFKYTAELSSDRYTYKDDSCERWGEMKRKR

SEQ ID NO: 71 - protein, 58 aa, synthetic construct
MLTAFFQIGSNHFFHEERQCKGKAHENEERYEERYEERYRDPFIHNVSNHIRETGKNL

SEQ ID NO: 72 - protein, 332 aa, synthetic construct
MRSLPHRQLRKTSARGSAAWPCSLPHRQLRKDAPPSIAMETCSLPHRQLRNARWARPRPGPRSLPHRQLRKDGSTFTLPASMFTAAQAAQKTPRAAWLSLALFTAAQAAQKACTTQFWEAMVFTAAQAAQKIRPISNSAGHSFTAAQAAQKTHFGSPVRAASFTAAQAAQKFSRYLLDTSNSFTAAQAAQKRRSEPRKWVNPFTAAQAAQKSRRPPSQADKSFTAAQAAQKTQEGYSAFTKEFTAAQAAQKASKPSRPSSPMFTAAQAAQKDGAVGDGAAQGFTAAQAAQKSKARSPKKTSPVHCRTGSSESHRIDARRHAVVHCRTGSSEN

SEQ ID NO: 73 - protein, 362 aa, synthetic construct
MEGDIRFDEILNPKWAFQSTPSAWRETESRGVLSVSERFQSTPSAWRETFRLRTSLDHQHISIHSLRMEGDCRKSCQNKYIAYFNPLPPHGGRLVFSLPGADLGSISIHSLRMEGDSSPDTAEPMLHDISIHSLRMEGDNVCDCPVCIHCISIHSLRMEGDIYRMLRIQNKIISIHSLRMEGDIYRMLRIQNKIISIHSLRMEGDPDTTTEVQTTEYFNPLPPHGGRQTNHYSETLTSPFQSTPSAWRETSSDWMSRRRPDISIHSLRMEGDVVSVAVSGKPLYFNPLPPHGGRQKTSRHPARESHFNPLPPHGGRRQVLAAVCLFQDISIHSLRMEGDIVMRVGFGYSAFISIHSLRMEGD

SEQ ID NO: 74 - protein, 359 aa, synthetic construct
MYFQLGFHYLCLKSNCEFPEQYKTITIGNQGTINGAILYQLVATNTNGLPVYTLDNDGGDVDAYKYANKSTGAKISKCFLKYSEKIGYNIYDAGLELFQNMSEEKNINEFRDYIDHFKYFVDKERSILELYSEMFDRFMAYDQKYRKNVPNMLTNILKRNYIITHLEFKSGQKKVGRGNKSTTKEMGLICIRDNGIESDDFTYKLADKSTVELPARGTNYLDGVAKLLYSPQSPPEKALAKTQEAKVASKNVNPKIGEKYQGVVKNINKHGIFVDFGAKKDGLVRYRNIPMKYKHDSTIVGFDKGDTIDVKLLDTNEKGFDLKILEDGELEFEKYKSEKINNTMGSVFKGIDLSFLPKQ

SEQ ID NO: 75 - protein, 362 aa, synthetic construct
MDTPNFSERIPVSLQSHPYYFAHYLNMARHNAYVILEYVNRELIKPGKNLDEDNLIQSTVLKDGYFDRKPDELSHRNRLLVQHFPFLREAENEGARTCNPVSYKLKTALAALNQWRNNASHYPLNQNHEKDFDLQPFFSFAIEACKKRMREVFQPDDFYLLETNEKQFYTLHNENGFTEKGLYCFICFFLEKKYAFQFLAGIKGFKNTTDNKFRATLETFTEHCCRLPKPKLDSSDIKLDMLGELSRCPAPLFDLLDIEERKKFIREPEEVKPDESGDREEVQQVLMKRYDDRFPYFALRYFEEKNLLKGISFHIHIGRWIKSEHTKKIMGAERDRRLLKDIRTFGELKEFSPEHEPYRYKT

SEQ ID NO: 76 - protein, 377 aa, synthetic construct
MGPWLCSHGRPAGMSRKRKQSLLQWGRGFAATEGRCRLKCWSCCSQLQWGRGFAATEGPGIPSTAGPLPRASMGPWLCSHGRLPASNGSAMPTPSFNGAVALQPRKEMPIAPSQRHCPTLQWGRGFAATEGSSGVPLCVGHRALQWGRGFAATEGAGRRWDTRGPAGFNGAVALQPRKGPTTSAVAWPPRSFNGAVALQPRKGAERLHVGPHRGASMGPWLCSHGRVSRCRRMGRSCMALQWGRGLAATEGRPSREQFAAAYKLQWGRGFAATEGNRLPAAAITAALLQWGRGFAATEGRPARPCCGARSSRFNGAVALQPRKVPANRSDVPNSSQLQWGRGFAATEGAPGRWAASATAGASMGPWLCSHGRKSQRS

SEQ ID NO: 77 - protein, 369 aa, synthetic construct
MKNKANDSRFGAGQSKKQKPITRAQQFMGGKHPEFAIFYNIAFFRLAGYINFLQGNSYEGNKKELEDIFIKWKSKVDDGVKVLGQTAYSKLRNYLWKAYMFEKNSSGFALEEKDINLINEMIFKLNHIRNFQSHIWHDNQPLVFSRELSAFIEEFHENAVMAQRKDNKLNYEVDDYLKEYCDSRKSRKPGRNDMRFFDIHDGKIMMTQDGRVFFLSLFLTRGEMNRFLQLKKGFKRNDLPKYKIKRRIFQHLTHRDGASRNYYGFEDNILDSLPKEQKSETLNIRQTFKLISYLNDLPIEVNNPEQFPIFLSNGKNVESSQEVIEWCKENDFLKELQFNLIKKEPKEGEAYLLDGSIECQLISQPDYSF

SEQ ID NO: 78 - protein, 313 aa, synthetic construct
MAVAPLPLSCSADAAKCSDPMKDVVDAALRISSLVDRVLAAHRLKLEAAERYPHLHVPTNDDLLGAILVGIAHAISDTDGLFEADMYDTIEDLYGGSGATDSATDDEDDTVWEEYERIQDVAYQQTCAWFKGVAPWIDPCDPVNADYRLVPVLVLSAIGPALGAWLSREIARLLESEDDAIFPLYGPLYGALICSGIGLDDAGAVGAFIRELKPRVDPWSAVGCDHRLFERHSRVVSRRLGEILGVSHAQLNVERIKAVADLDQMRSAIWHQRQQSDRKLKMGQLVSKVGSERARWLVEKARFKARAKACVGR

SEQ ID NO: 79 - DNA, 24 bp, synthetic construct
  source 1..24: mol_type = other DNA
tcttcaaattgtgatacgtcccaa

SEQ ID NO: 80 - DNA, 24 bp, synthetic construct
  source 1..24: mol_type = other DNA
ttgggacgtatcacaatttgaaga

SEQ ID NO: 81 - DNA, 36 bp, synthetic construct
  source 1..36: mol_type = other DNA
gtttccattcaattaattgcctctattaaaagagac

SEQ ID NO: 82 - DNA, 28 bp, synthetic construct
  source 1..28: mol_type = other DNA
gtcgtccccgcgcccgcgggggttgctc

SEQ ID NO: 83 - DNA, 29 bp, synthetic construct
  source 1..29: mol_type = other DNA
gtcgtccccgcgcccgcgggggttgctcc

SEQ ID NO: 84 - DNA, 29 bp, synthetic construct
  source 1..29: mol_type = other DNA
ggagcaacccccgcgggcgcggggacgac

SEQ ID NO: 85 - DNA, 28 bp, synthetic construct
  source 1..28: mol_type = other DNA
gtttgccatcgcccagatggtttagaag

SEQ ID NO: 86 - DNA, 28 bp, synthetic construct
  source 1..28: mol_type = other DNA
cttctaaaccatctgggcgatggcaaac

SEQ ID NO: 87 - DNA, 30 bp, synthetic construct
  source 1..30: mol_type = other DNA
gtcctcatcgcccctacgaggggtcgcaac

SEQ ID NO: 88 - DNA, 30 bp, synthetic construct
  source 1..30: mol_type = other DNA
gttgcgacccctcgtaggggcgatgaggac

SEQ ID NO: 89 - DNA, 28 bp, synthetic construct
  source 1..28: mol_type = other DNA
gttcactgccgcgtaggcagctcagaaa

SEQ ID NO: 90 - DNA, 28 bp, synthetic construct
  source 1..28: mol_type = other DNA
tttctgagctgcctacgcggcagtgaac

SEQ ID NO: 91 - DNA, 37 bp, synthetic construct
  source 1..37: mol_type = other DNA
gtggcggtcgcccctcggggggaccgaggatcgcaac

SEQ ID NO: 92 - DNA, 37 bp, synthetic construct
  source 1..37: mol_type = other DNA
gttgcgatcctcggtccccccgaggggcgaccgccac

SEQ ID NO: 93 - DNA, 29 bp, synthetic construct
  source 1..29: mol_type = other DNA
gttctctccgcgcgagcggaggtggtccg

SEQ ID NO: 94 - DNA, 29 bp, synthetic construct
  source 1..29: mol_type = other DNA
cggaccacctccgctcgcgcggagagaac

SEQ ID NO: 95 - DNA, 36 bp, synthetic construct
  source 1..36: mol_type = other DNA
gttgtaattgctcttattttgaagggtatacacaac

SEQ ID NO: 96 - DNA, 36 bp, synthetic construct
  source 1..36: mol_type = other DNA
gttgtgtatacccttcaaaataagagcaattacaac

SEQ ID NO: 97 - DNA, 36 bp, synthetic construct
  source 1..36: mol_type = other DNA
gctgtactcacccttcaaataaagggcttttacagc

SEQ ID NO: 98 - DNA, 36 bp, synthetic construct
  source 1..36: mol_type = other DNA
gctgtaaaagccctttatttgaagggtgagtacagc

SEQ ID NO: 99 - DNA, 36 bp, synthetic construct
  source 1..36: mol_type = other DNA
gttgggaatacccttagttagaagggtggagacaac

SEQ ID NO: 100 - DNA, 37 bp, synthetic construct
  source 1..37: mol_type = other DNA
gttgggaatacccttagttagaagggtggagacaact

SEQ ID NO: 101 - DNA, 36 bp, synthetic construct; identical to SEQ ID NO: 99
  source 1..36: mol_type = other DNA

SEQ ID NO: 102 - DNA, 36 bp, synthetic construct
  source 1..36: mol_type = other DNA
gttgtctccacccttctaactaagggtattcccaac

SEQ ID NO: 103 - DNA, 36 bp, synthetic construct
  source 1..36: mol_type = other DNA
gttgtagttccctgatcgttcttggtatggtataat

SEQ ID NO: 104 - DNA, 36 bp, synthetic construct
  source 1..36: mol_type = other DNA
attataccataccaagaacgatcagggaactacaac

SEQ ID NO: 105 - DNA, 36 bp, synthetic construct
  source 1..36: mol_type = other DNA
aggatagcagttcagaaatcgcggtccagctgcaac

SEQ ID NO: 106 - DNA, 36 bp, synthetic construct
  source 1..36: mol_type = other DNA
gttgcagctggaccgcgatttctgaactgctatcct

SEQ ID NO: 107 - DNA, 29 bp, synthetic construct
  source 1..29: mol_type = other DNA
gggctcatccccgcacgcgcggggagcac

SEQ ID NO: 108 - DNA, 29 bp, synthetic construct
  source 1..29: mol_type = other DNA
gtgctccccgcgcgtgcggggatgagccc

SEQ ID NO: 109 - DNA, 31 bp, synthetic construct
  source 1..31: mol_type = other DNA
agtcttccccacatgggtgggggtgtttcta

SEQ ID NO: 110 - DNA, 30 bp, synthetic construct
  source 1..30: mol_type = other DNA
gtcttccccacatgggtgggggtgtttcta

SEQ ID NO: 111 - DNA, 36 bp, synthetic construct
  source 1..36: mol_type = other DNA
gttgcaaaggctgtccctcggtagagggattgaaac

SEQ ID NO: 112 - DNA, 38 bp, synthetic construct
  source 1..38: mol_type = other DNA
gttgcaaaggctgtccctcggtagagggattgaaacac

SEQ ID NO: 113 - DNA, 29 bp, synthetic construct
  source 1..29: mol_type = other DNA
cggaccatccccacgcacgtggggagaac

SEQ ID NO: 114 - DNA, 29 bp, synthetic construct
  source 1..29: mol_type = other DNA
gttctccccacgtgcgtggggatggtccg

SEQ ID NO: 115 - DNA, 36 bp, synthetic construct
  source 1..36: mol_type = other DNA
gtccgctttcatctaggaagtggaattaatggaaac

SEQ ID NO: 116 - DNA, 36 bp, synthetic construct
  source 1..36: mol_type = other DNA
gtttccattaattccacttcctagatgaaagcggac

SEQ ID NO: 117 - DNA, 36 bp, synthetic construct
  source 1..36: mol_type = other DNA
gtcgcagctccttcgggagctgctcttcattgaggc

SEQ ID NO: 118 - DNA, 36 bp, synthetic construct
  source 1..36: mol_type = other DNA
gcctcaatgaagagcagctcccgaaggagctgcgac

SEQ ID NO: 119 - DNA, 33 bp, synthetic construct
  source 1..33: mol_type = other DNA
gtcgcgccccgcacggggcgcgtggattgaaac

SEQ ID NO: 120 - DNA, 31 bp, synthetic construct
  source 1..31: mol_type = other DNA
gtttcaatccacgcgccccgtgcggggcgcg

SEQ ID NO: 121 - DNA, 36 bp, synthetic construct
  source 1..36: mol_type = other DNA
ggtgtgaaagccatctttttgtatggtagggacacc

SEQ ID NO: 122 - DNA, 36 bp, synthetic construct
  source 1..36: mol_type = other DNA
ggtgtccctaccatacaaaaagatggctttcacacc

SEQ ID NO: 123 - DNA, 33 bp, synthetic construct
  source 1..33: mol_type = other DNA
gtcgctcccctcgcgggagcgtggattgaaata

SEQ ID NO: 124 - DNA, 33 bp, synthetic construct
  source 1..33: mol_type = other DNA
tatttcaatccacgctcccgcgaggggagcgac

SEQ ID NO: 125 - DNA, 34 bp, synthetic construct
  source 1..34: mol_type = other DNA
aaataccacccaagaatgagggggttctataacc

SEQ ID NO: 126 - DNA, 34 bp, synthetic construct
  source 1..34: mol_type = other DNA
ggttatagaaccccctcattcttgggtggtattt

SEQ ID NO: 127 - DNA, 36 bp, synthetic construct
  source 1..36: mol_type = other DNA
agtttatccgatgggagatcggggaggaaccgcaac

SEQ ID NO: 128 - DNA, 36 bp, synthetic construct
  source 1..36: mol_type = other DNA
gttgcggttcctccccgatctcccatcggataaact

SEQ ID NO: 129 - DNA, 35 bp, synthetic construct
  source 1..35: mol_type = other DNA
cttccaatttgcgcgtgggcgtgagttgggggcac

SEQ ID NO: 130 - DNA, 35 bp, synthetic construct
  source 1..35: mol_type = other DNA
gtgcccccaactcacgcccacgcgcaaattggaag

SEQ ID NO: 131 - DNA, 30 bp, synthetic construct
  source 1..30: mol_type = other DNA
gtcgcagtcctcactaaaattggacatgac

SEQ ID NO: 132 - DNA, 30 bp, synthetic construct
  source 1..30: mol_type = other DNA
gtcatgtccaattttagtgaggactgcgac

SEQ ID NO: 133 - DNA, 36 bp, synthetic construct
  source 1..36: mol_type = other DNA
gctgtgatagacctcgatttgtggggtagtaacagc

SEQ ID NO: 134 - DNA, 36 bp, synthetic construct
  source 1..36: mol_type = other DNA
gctgttactaccccacaaatcgaggtctatcacagc

SEQ ID NO: 135 - DNA, 31 bp, synthetic construct
  source 1..31: mol_type = other DNA
tgaatacagctcgatatagtgagcaataact

SEQ ID NO: 136 - DNA, 31 bp, synthetic construct
  source 1..31: mol_type = other DNA
agttattgctcactatatcgagctgtattca

SEQ ID NO: 137 - DNA, 36 bp, synthetic construct
  source 1..36: mol_type = other DNA
gtttcaccagccgattttttaaacggtaactgaaac

SEQ ID NO: 138 - DNA, 36 bp, synthetic construct
  source 1..36: mol_type = other DNA
gtttcagttaccgtttaaaaaatcggctggtgaaac

SEQ ID NO: 139 - DNA, 36 bp, synthetic construct
  source 1..36: mol_type = other DNA
gttgtagaagccacttgtttgaaatggcatgacaac

SEQ ID NO: 140 - DNA, 36 bp, synthetic construct
  source 1..36: mol_type = other DNA
gttgtcatgccatttcaaacaagtggcttctacaac

SEQ ID NO: 141 - DNA, 36 bp, synthetic construct
  source 1..36: mol_type = other DNA
gttggagatcacccccaaatcgagggggactgcacc

SEQ ID NO: 142 - DNA, 36 bp, synthetic construct
  source 1..36: mol_type = other DNA
ggtgcagtccccctcgatttgggggtgatctccaac

SEQ ID NO: 143 - DNA, 37 bp, synthetic construct
  source 1..37: mol_type = other DNA
gaatcgcccggcttcccagccgggcgcggattgaaac

SEQ ID NO: 144 - DNA, 37 bp, synthetic construct
  source 1..37: mol_type = other DNA
gtttcaatccgcgcccggctgggaagccgggcgattc

SEQ ID NO: 145 - DNA, 20 bp, synthetic construct
  source 1..20: mol_type = other DNA
gttcaattttgagtactata

SEQ ID NO: 146 - DNA, 20 bp, synthetic construct
  source 1..20: mol_type = other DNA
tatagtactcaaaattgaac

SEQ ID NO: 147 - DNA, 24 bp, synthetic construct
  source 1..24: mol_type = other DNA
gagcatacgcacaaagtccacagt

SEQ ID NO: 148 - DNA, 24 bp, synthetic construct
  source 1..24: mol_type = other DNA
actgtggactttgtgcgtatgctc

SEQ ID NO: 149 - DNA, 36 bp, synthetic construct
  source 1..36: mol_type = other DNA
gttttagagctgtgctgtttcgaatggttccaaaac

SEQ ID NO: 150 - DNA, 36 bp, synthetic construct
  source 1..36: mol_type = other DNA
gttttggaaccattcgaaacagcacagctctaaaac

SEQ ID NO: 151 - DNA, 36 bp, synthetic construct; identical to SEQ ID NO: 149
  source 1..36: mol_type = other DNA

SEQ ID NO: 152 - DNA, 36 bp, synthetic construct; identical to SEQ ID NO: 150
  source 1..36: mol_type = other DNA

SEQ ID NO: 153 - DNA, 27 bp, synthetic construct
  source 1..27: mol_type = other DNA
ggttcacccgcgcacgcgcgtgtaagg

SEQ ID NO: 154 - DNA, 27 bp, synthetic construct
  source 1..27: mol_type = other DNA
ccttacacgcgcgtgcgcgggtgaacc

SEQ ID NO: 155 - DNA, 33 bp, synthetic construct
  source 1..33: mol_type = other DNA
gtctccctccatgcggagggagtggattgaaat

SEQ ID NO: 156 - DNA, 33 bp, synthetic construct
  source 1..33: mol_type = other DNA
atttcaatccactccctccgcatggagggagac

SEQ ID NO: 157 - DNA, 36 bp, synthetic construct
  source 1..36: mol_type = other DNA
gttgtagttccctttcattttgggatcattcacacc

SEQ ID NO: 158 - DNA, 36 bp, synthetic construct
  source 1..36: mol_type = other DNA
ggtgtgaatgatcccaaaatgaaagggaactacaac

SEQ ID NO: 159 - DNA, 36 bp, synthetic construct
  source 1..36: mol_type = other DNA
gttgtagaagcctatcgtttggataggtatgacaac

SEQ ID NO: 160 - DNA, 36 bp, synthetic construct
  source 1..36: mol_type = other DNA
gttgtcatacctatccaaacgataggcttctacaac

SEQ ID NO: 161 - DNA, 28 bp, synthetic construct
  source 1..28: mol_type = other DNA
gttcgctgccgcgcaggcagctcagaaa

SEQ ID NO: 162 - DNA, 28 bp, synthetic construct
  source 1..28: mol_type = other DNA
tttctgagctgcctgcgcggcagcgaac

SEQ ID NO: 163 - DNA, 37 bp, synthetic construct
  source 1..37: mol_type = other DNA
gttgcaccgaccacgcccactgaagggcgactgcacc

SEQ ID NO: 164 - DNA, 37 bp, synthetic construct
  source 1..37: mol_type = other DNA
ggtgcagtcgcccttcagtgggcgtggtcggtgcaac

SEQ ID NO: 165 - DNA, 33 bp, synthetic construct
  source 1..33: mol_type = other DNA
gtcgctccccattcggggagcgtggattgaaat

SEQ ID NO: 166 - DNA, 33 bp, synthetic construct
  source 1..33: mol_type = other DNA
atttcaatccacgctccccgaatggggagcgac

SEQ ID NO: 167 - DNA, 36 bp, synthetic construct
  source 1..36: mol_type = other DNA
gttgtagaagccctcattttgagagggtataacaac

SEQ ID NO: 168 - DNA, 36 bp, synthetic construct
  source 1..36: mol_type = other DNA
gttgttataccctctcaaaatgagggcttctacaac

SEQ ID NO: 169 - DNA, 36 bp, synthetic construct
  source 1..36: mol_type = other DNA
gttttagatataagtcattttaagtacatagaaccc

SEQ ID NO: 170 - DNA, 36 bp, synthetic construct
  source 1..36: mol_type = other DNA
gggttctatgtacttaaaatgacttatatctaaaac

SEQ ID NO: 171 - DNA, 36 bp, synthetic construct
  source 1..36: mol_type = other DNA
gtggcgacgggtgaggaggccggatcgggttggagg

SEQ ID NO: 172 - DNA, 36 bp, synthetic construct
  source 1..36: mol_type = other DNA
cctccaacccgatccggcctcctcacccgtcgccac

SEQ ID NO: 173 - DNA, 30 bp, synthetic construct
  source 1..30: mol_type = other DNA
gtttttatcgtccctataaggggttgaaac

SEQ ID NO: 174 - DNA, 30 bp, synthetic construct
  source 1..30: mol_type = other DNA
gtttcaaccccttatagggacgataaaaac

SEQ ID NO: 175 - DNA, 36 bp, synthetic construct; identical to SEQ ID NO: 157
  source 1..36: mol_type = other DNA

SEQ ID NO: 176 - DNA, 36 bp, synthetic construct; identical to SEQ ID NO: 158
  source 1..36: mol_type = other DNA

SEQ ID NO: 177 - DNA, 37 bp, synthetic construct
  source 1..37: mol_type = other DNA
gcccccaacaaaccatcagccgaaaggcgattgagac

SEQ ID NO: 178 - DNA, 37 bp, synthetic construct
  source 1..37: mol_type = other DNA
gtctcaatcgcctttcggctgatggtttgttgggggc

SEQ ID NO: 179 - DNA, 36 bp, synthetic construct; identical to SEQ ID NO: 141
  source 1..36: mol_type = other DNA

SEQ ID NO: 180 - DNA, 36 bp, synthetic construct; identical to SEQ ID NO: 142
  source 1..36: mol_type = other DNA

SEQ ID NO: 181 - DNA, 33 bp, synthetic construct
  source 1..33: mol_type = other DNA
gtcgaggctcgcgagagccttgtggattgaaat

SEQ ID NO: 182 - DNA, 33 bp, synthetic construct
  source 1..33: mol_type = other DNA
atttcaatccacaaggctctcgcgagcctcgac

SEQ ID NO: 183 - DNA, 33 bp, synthetic construct
  source 1..33: mol_type = other DNA
gtcgccttccccccggaaggcgtggattgaaac

SEQ ID NO: 184 - DNA, 33 bp, synthetic construct
  source 1..33: mol_type = other DNA
gtttcaatccacgccttccggggggaaggcgac

SEQ ID NO: 185 - DNA, 29 bp, synthetic construct
  source 1..29: mol_type = other DNA
gtttgccccgcatgtgcggggatgatccg

SEQ ID NO: 186 - DNA, 29 bp, synthetic construct
  source 1..29: mol_type = other DNA
cggatcatccccgcacatgcggggcaaac

SEQ ID NO: 187 - DNA, 23 bp, synthetic construct
  source 1..23: mol_type = other DNA
ctccttctgctcaggcgtggctt

SEQ ID NO: 188 - DNA, 23 bp, synthetic construct
  source 1..23: mol_type = other DNA
aagccacgcctgagcagaaggag

SEQ ID NO: 189 - DNA, 37 bp, synthetic construct
  source 1..37: mol_type = other DNA
cgtttccacggcatcacagccgtggccgaattgaagc

SEQ ID NO: 190 - DNA, 37 bp, synthetic construct
  source 1..37: mol_type = other DNA
gcttcaattcggccacggctgtgatgccgtggaaacg

SEQ ID NO: 191 - DNA, 37 bp, synthetic construct
  source 1..37: mol_type = other DNA
gtaagaatcaaataatcccgatacgcgggattaagac

SEQ ID NO: 192 - DNA, 37 bp, synthetic construct
  source 1..37: mol_type = other DNA
gtcttaatcccgcgtatcgggattatttgattcttac

SEQ ID NO: 193 - DNA, 32 bp, synthetic construct
  source 1..32: mol_type = other DNA
gctgcattccccgcgcgagaggggattgagac

SEQ ID NO: 194 - DNA, 32 bp, synthetic construct
  source 1..32: mol_type = other DNA
gtctcaatcccctctcgcgcggggaatgcagc

SEQ ID NO: 195 - DNA, 36 bp, synthetic construct
  source 1..36: mol_type = other DNA
gttgtgtgtacccttcgaatagagggtagatccaac

SEQ ID NO: 196 - DNA, 36 bp, synthetic construct
  source 1..36: mol_type = other DNA
gttggatctaccctctattcgaagggtacacacaac

SEQ ID NO: 197 - DNA, 31 bp, synthetic construct
  source 1..31: mol_type = other DNA
gtcgcgccttcgcgggcgcgtgagttgaaac

SEQ ID NO: 198 - DNA, 31 bp, synthetic construct
  source 1..31: mol_type = other DNA
gtttcaactcacgcgcccgcgaaggcgcgac

SEQ ID NO: 199 - DNA, 28 bp, synthetic construct
  source 1..28: mol_type = other DNA
ggttcccccgtacacgcggggatagacc

SEQ ID NO: 200 - DNA, 28 bp, synthetic construct
  source 1..28: mol_type = other DNA
ggtctatccccgcgtgtacgggggaacc

SEQ ID NO: 201 - DNA, 29 bp, synthetic construct
  source 1..29: mol_type = other DNA
gtgctccccgcacacgcggggatgatccc

SEQ ID NO: 202 - DNA, 29 bp, synthetic construct
  source 1..29: mol_type = other DNA
gggatcatccccgcgtgtgcggggagcac

SEQ ID NO: 203 - DNA, 36 bp, synthetic construct
  source 1..36: mol_type = other DNA
ggtggagacacgcggatttaggggtgtgatgacagg

SEQ ID NO: 204 - DNA, 36 bp, synthetic construct
  source 1..36: mol_type = other DNA
cctgtcatcacacccctaaatccgcgtgtctccacc

SEQ ID NO: 205 - DNA, 35 bp, synthetic construct
  source 1..35: mol_type = other DNA
attcctaagcttttacgcttaggacttcattgagg

SEQ ID NO: 206 - DNA, 35 bp, synthetic construct
  source 1..35: mol_type = other DNA
cctcaatgaagtcctaagcgtaaaagcttaggaat

SEQ ID NO: 207 - DNA, 36 bp, synthetic construct
  source 1..36: mol_type = other DNA
ccctcaactattgaaacgtgtttcagtcgtttcagg

SEQ ID NO: 208 - DNA, 36 bp, synthetic construct
  source 1..36: mol_type = other DNA
cctgaaacgactgaaacacgtttcaatagttgaggg

SEQ ID NO: 209 - DNA, 36 bp, synthetic construct
  source 1..36: mol_type = other DNA
ggtttccgtccccgtgaaggggaagttgtatgaaac

SEQ ID NO: 210 - DNA, 36 bp, synthetic construct
  source 1..36: mol_type = other DNA
gtttcatacaacttccccttcacggggacggaaacc

SEQ ID NO: 211 - DNA, 41 bp, synthetic construct
  source 1..41: mol_type = other DNA
ttatgtgctcagggccactgcatggtgctgatggaggccac

SEQ ID NO: 212 - DNA, 41 bp, synthetic construct
  source 1..41: mol_type = other DNA
gtggcctccatcagcaccatgcagtggccctgagcacataa

SEQ ID NO: 213 - DNA, 36 bp, synthetic construct
  source 1..36: mol_type = other DNA
ggtgtcggaaaccgctaattcaggggccgctacaac

SEQ ID NO: 214 - DNA, 36 bp, synthetic construct
  source 1..36: mol_type = other DNA
gttgtagcggcccctgaattagcggtttccgacacc

SEQ ID NO: 215 - DNA, 36 bp, synthetic construct
  source 1..36: mol_type = other DNA
agtttagcagattgggatttgtactctgaccggaac

SEQ ID NO: 216 - DNA, 36 bp, synthetic construct
  source 1..36: mol_type = other DNA
gttccggtcagagtacaaatcccaatctgctaaact

SEQ ID NO: 217 - DNA, 37 bp, synthetic construct
  source 1..37: mol_type = other DNA
gtagaaatgagtacaaagcgatagagagcttaataac

SEQ ID NO: 218 - DNA, 37 bp, synthetic construct
  source 1..37: mol_type = other DNA
gttattaagctctctatcgctttgtactcatttctac

SEQ ID NO: 219 - DNA, 32 bp, synthetic construct
  source 1..32: mol_type = other DNA
aactcggaaggattcagaagaagctttcatct

SEQ ID NO: 220 - DNA, 32 bp, synthetic construct
  source 1..32: mol_type = other DNA
agatgaaagcttcttctgaatccttccgagtt

SEQ ID NO: 221 - DNA, 28 bp, synthetic construct
  source 1..28: mol_type = other DNA
gttcactgccgcacaggcagctcagaaa

SEQ ID NO: 222 - DNA, 28 bp, synthetic construct
  source 1..28: mol_type = other DNA
tttctgagctgcctgtgcggcagtgaac

SEQ ID NO: 223 - DNA, 33 bp, synthetic construct; identical to SEQ ID NO: 155
  source 1..33: mol_type = other DNA

SEQ ID NO: 224 - DNA, 33 bp, synthetic construct; identical to SEQ ID NO: 156
  source 1..33: mol_type = other DNA

SEQ ID NO: 225 - DNA, 36 bp, synthetic construct
  source 1..36: mol_type = other DNA
gttgaaagagaatagcccgacatagtgggcaatcaa

SEQ ID NO: 226 - DNA, 36 bp, synthetic construct
  source 1..36: mol_type = other DNA
ttgattgcccactatgtcgggctattctctttcaac

SEQ ID NO: 227 - DNA, 29 bp, synthetic construct
  source 1..29: mol_type = other DNA
gttgttctcaccttccaaaattaaggcat

SEQ ID NO: 228 - DNA, 29 bp, synthetic construct
  source 1..29: mol_type = other DNA
atgccttaattttggaaggtgagaacaac

SEQ ID NO: 229 - DNA, 36 bp, synthetic construct
  source 1..36: mol_type = other DNA
ccttccgtggctgcaaagccacggccccattgaagc

SEQ ID NO: 230 - DNA, 36 bp, synthetic construct
  source 1..36: mol_type = other DNA
gcttcaatggggccgtggctttgcagccacggaagg

SEQ ID NO: 231 - DNA, 36 bp, synthetic construct
  source 1..36: mol_type = other DNA
ggtgtggatgcctctattttgagaggtagaatcacc

SEQ ID NO: 232 - DNA, 36 bp, synthetic construct
  source 1..36: mol_type = other DNA
ggtgattctacctctcaaaatagaggcatccacacc

SEQ ID NO: 233 - DNA, 39 bp, synthetic construct
  source 1..39: mol_type = other DNA
gtcgcagctacaaggccgccgcaatggccattggaacat

SEQ ID NO: 234 - DNA, 39 bp, synthetic construct
  source 1..39: mol_type = other DNA
atgttccaatggccattgcggcggccttgtagctgcgac

SEQ ID NO: 235 - DNA, 73 bp, synthetic construct
  source 1..73: mol_type = other DNA
cttgtggaaaggacgaaacaccggttcaattatgagtactatacaaatgctggtaacactgtggtccacaagg

SEQ ID NO: 236 - DNA, 74 bp, synthetic construct
  source 1..74: mol_type = other DNA
acgcacactggacgcgcaaaaaaatatagtactcataattgaacccttgtggaccacagtgttaccagcatttg

SEQ ID NO: 237 - DNA, 73 bp, synthetic construct
  source 1..73: mol_type = other DNA
cttgtggaaaggacgaaacaccggttcaattatgagtactatagtgcagctcctcagtcacaatcagggaagc

SEQ ID NO: 238 - DNA, 74 bp, synthetic construct
  source 1..74: mol_type = other DNA
acgcacactggacgcgcaaaaaaatatagtactcataattgaacgcttccctgattgtgactgaggagctgcac

SEQ ID NO: 239 - DNA, 73 bp, synthetic construct
  source 1..73: mol_type = other DNA
cttgtggaaaggacgaaacaccgtatagtactcaaaattgaaccaaatgctggtaacactgtggtccacaagg

SEQ ID NO: 240 - DNA, 74 bp, synthetic construct
  source 1..74: mol_type = other DNA
acgcacactggacgcgcaaaaaaagttcaattttgagtactataccttgtggaccacagtgttaccagcatttg

SEQ ID NO: 241 - DNA, 73 bp, synthetic construct
  source 1..73: mol_type = other DNA
cttgtggaaaggacgaaacaccgtatagtactcaaaattgaacgtgcagctcctcagtcacaatcagggaagc

SEQ ID NO: 242 - DNA, 74 bp, synthetic construct
  source 1..74: mol_type = other DNA
acgcacactggacgcgcaaaaaaagttcaattttgagtactatagcttccctgattgtgactgaggagctgcac

SEQ ID NO: 243 - DNA, 77 bp, synthetic construct
  source 1..77: mol_type = other DNA
cttgtggaaaggacgaaacaccggagcatacgcacaaagtccacagtcaaatgctggtaacactgtggtccacaagg

SEQ ID NO: 244 - DNA, 78 bp, synthetic construct
  source 1..78: mol_type = other DNA
acgcacactggacgcgcaaaaaaaactgtggactttgtgcgtatgctcccttgtggaccacagtgttaccagcatttg

SEQ ID NO: 245 - DNA, 77 bp, synthetic construct
  source 1..77: mol_type = other DNA
cttgtggaaaggacgaaacaccggagcatacgcacaaagtccacagtgtgcagctcctcagtcacaatcagggaagc

SEQ ID NO: 246 - DNA, 78 bp, synthetic construct
  source 1..78: mol_type = other DNA
acgcacactggacgcgcaaaaaaaactgtggactttgtgcgtatgctcgcttccctgattgtgactgaggagctgcac

SEQ ID NO: 247 - DNA, 77 bp, synthetic construct
  source 1..77: mol_type = other DNA
cttgtggaaaggacgaaacaccgactgtggactttgtgcgtatgctccaaatgctggtaacactgtggtccacaagg

SEQ ID NO: 248 - DNA, 78 bp, synthetic construct
  source 1..78: mol_type = other DNA
acgcacactggacgcgcaaaaaaagagcatacgcacaaagtccacagtccttgtggaccacagtgttaccagcatttg

SEQ ID NO: 249 - DNA, 77 bp, synthetic construct
  source 1..77: mol_type = other DNA
cttgtggaaaggacgaaacaccgactgtggactttgtgcgtatgctcgtgcagctcctcagtcacaatcagggaagc

SEQ ID NO: 250 - DNA, 78 bp, synthetic construct
  source 1..78: mol_type = other DNA
acgcacactggacgcgcaaaaaaagagcatacgcacaaagtccacagtgcttccctgattgtgactgaggagctgcac

SEQ ID NO: 251 - DNA, 89 bp, synthetic construct
  source 1..89: mol_type = other DNA
cttgtggaaaggacgaaacaccggtttaagagctgtgctgtttcgaatggttcctaaaccaaatgctggtaacactgtggtccacaagg

SEQ ID NO: 252 - DNA, 90 bp, synthetic construct
  source 1..90: mol_type = other DNA
acgcacactggacgcgcaaaaaaagtttaggaaccattcgaaacagcacagctcttaaacccttgtggaccacagtgttaccagcatttg

SEQ ID NO: 253 - DNA, 89 bp, synthetic construct
  source 1..89: mol_type = other DNA
cttgtggaaaggacgaaacaccggtttaagagctgtgctgtttcgaatggttcctaaacgtgcagctcctcagtcacaatcagggaagc

SEQ ID NO: 254 - DNA, 90 bp, synthetic construct
  source 1..90: mol_type = other DNA
acgcacactggacgcgcaaaaaaagtttaggaaccattcgaaacagcacagctcttaaacgcttccctgattgtgactgaggagctgcac

SEQ ID NO: 255 - DNA, 89 bp, synthetic construct; identical to SEQ ID NO: 251
  source 1..89: mol_type = other DNA

SEQ ID NO: 256 - DNA, 90 bp, synthetic construct; identical to SEQ ID NO: 252
  source 1..90: mol_type = other DNA

SEQ ID NO: 257 - DNA, 89 bp, synthetic construct; identical to SEQ ID NO: 253
  source 1..89: mol_type = other DNA

SEQ ID NO: 258 - DNA, 90 bp, synthetic construct; identical to SEQ ID NO: 254
  source 1..90: mol_type = other DNA

SEQ ID NO: 259 - DNA, 80 bp, synthetic construct
  source 1..80: mol_type = other DNA
cttgtggaaaggacgaaacaccgggttcacccgcgcacgcgcgtgtaaggcaaatgctggtaacactgtggtccacaagg

SEQ ID NO: 260 - DNA, 81 bp, synthetic construct
  source 1..81: mol_type = other DNA
acgcacactggacgcgcaaaaaaaccttacacgcgcgtgcgcgggtgaaccccttgtggaccacagtgttaccagcatttg

SEQ ID NO: 261 - DNA, 80 bp, synthetic construct
  source 1..80: mol_type = other DNA
cttgtggaaaggacgaaacaccgggttcacccgcgcacgcgcgtgtaagggtgcagctcctcagtcacaatcagggaagc

SEQ ID NO: 262 - DNA, 81 bp, synthetic construct
  source 1..81: mol_type = other DNA
acgcacactggacgcgcaaaaaaaccttacacgcgcgtgcgcgggtgaaccgcttccctgattgtgactgaggagctgcac

SEQ ID NO: 263 - DNA, 80 bp, synthetic construct
  source 1..80: mol_type = other DNA
cttgtggaaaggacgaaacaccgccttacacgcgcgtgcgcgggtgaacccaaatgctggtaacactgtggtccacaagg

SEQ ID NO: 264 - DNA, 81 bp, synthetic construct
  source 1..81: mol_type = other DNA
acgcacactggacgcgcaaaaaaaggttcacccgcgcacgcgcgtgtaaggccttgtggaccacagtgttaccagcatttg

SEQ ID NO: 265 - DNA, 80 bp, synthetic construct
  source 1..80: mol_type = other DNA
cttgtggaaaggacgaaacaccgccttacacgcgcgtgcgcgggtgaaccgtgcagctcctcagtcacaatcagggaagc

SEQ ID NO: 266 - DNA, 81 bp, synthetic construct
  source 1..81: mol_type = other DNA
acgcacactggacgcgcaaaaaaaggttcacccgcgcacgcgcgtgtaagggcttccctgattgtgactgaggagctgcac

SEQ ID NO: 267 - DNA, 86 bp, synthetic construct
  source 1..86: mol_type = other DNA
cttgtggaaaggacgaaacaccggtctccctccatgcggagggagtggattgaaatcaaatgctggtaacactgtggtccacaagg

SEQ ID NO: 268 - DNA, 87 bp, synthetic construct
  source 1..87: mol_type = other DNA
acgcacactggacgcgcaaaaaaaatttcaatccactccctccgcatggagggagacccttgtggaccacagtgttaccagcatttg

SEQ ID NO: 269 - DNA, 86 bp, synthetic construct
  source 1..86: mol_type = other DNA
cttgtggaaaggacgaaacaccggtctccctccatgcggagggagtggattgaaatgtgcagctcctcagtcacaatcagggaagc

SEQ ID NO: 270 - DNA, 87 bp, synthetic construct
  source 1..87: mol_type = other DNA
acgcacactggacgcgcaaaaaaaatttcaatccactccctccgcatggagggagacgcttccctgattgtgactgaggagctgcac

SEQ ID NO: 271 - DNA, 89 bp, synthetic construct
  source 1..89: mol_type = other DNA
cttgtggaaaggacgaaacaccggttgtagttccctttcatttcgggatcattcacacccaaatgctggtaacactgtggtccacaagg

SEQ ID NO: 272 - DNA, 90 bp, synthetic construct
  source 1..90: mol_type = other DNA
acgcacactggacgcgcaaaaaaaggtgtgaatgatcccgaaatgaaagggaactacaacccttgtggaccacagtgttaccagcatttg

SEQ ID NO: 273 - DNA, 89 bp, synthetic construct
  source 1..89: mol_type = other DNA
cttgtggaaaggacgaaacaccggttgtagttccctttcatttcgggatcattcacaccgtgcagctcctcagtcacaatcagggaagc

SEQ ID NO: 274 - DNA, 90 bp, synthetic construct
  source 1..90: mol_type = other DNA
acgcacactggacgcgcaaaaaaaggtgtgaatgatcccgaaatgaaagggaactacaacgcttccctgattgtgactgaggagctgcac

SEQ ID NO: 275 - DNA, 89 bp, synthetic construct
  source 1..89: mol_type = other DNA
cttgtggaaaggacgaaacaccggttgtagaagcctatcgtttggataggtatgacaaccaaatgctggtaacactgtggtccacaagg

SEQ ID NO: 276 - DNA, 90 bp, synthetic construct
  source 1..90: mol_type = other DNA
acgcacactggacgcgcaaaaaaagttgtcatacctatccaaacgataggcttctacaacccttgtggaccacagtgttaccagcatttg

SEQ ID NO: 277 - DNA, 89 bp, synthetic construct
  source 1..89: mol_type = other DNA
cttgtggaaaggacgaaacaccggttgtagaagcctatcgtttggataggtatgacaacgtgcagctcctcagtcacaatcagggaagc

SEQ ID NO: 278 - DNA, 90 bp, synthetic construct
  source 1..90: mol_type = other DNA
acgcacactggacgcgcaaaaaaagttgtcatacctatccaaacgataggcttctacaacgcttccctgattgtgactgaggagctgcac

SEQ ID NO: 279 - DNA, 81 bp, synthetic construct
  source 1..81: mol_type = other DNA
cttgtggaaaggacgaaacaccggttcgctgccgcgcaggcagctcagaaacaaatgctggtaacactgtggtccacaagg

SEQ ID NO: 280 - DNA, 82 bp, synthetic construct
  source 1..82: mol_type = other DNA
acgcacactggacgcgcaaaaaaatttctgagctgcctgcgcggcagcgaacccttgtggaccacagtgttaccagcatttg

SEQ ID NO: 281 - DNA, 81 bp, synthetic construct
  source 1..81: mol_type = other DNA
cttgtggaaaggacgaaacaccggttcgctgccgcgcaggcagctcagaaagtgcagctcctcagtcacaatcagggaagc

SEQ ID NO: 282 - DNA, 82 bp, synthetic construct
  source 1..82: mol_type = other DNA
acgcacactggacgcgcaaaaaaatttctgagctgcctgcgcggcagcgaacgcttccctgattgtgactgaggagctgcac

SEQ ID NO: 283 - DNA, 90 bp, synthetic construct
  source 1..90: mol_type = other DNA
cttgtggaaaggacgaaacaccggttgcaccgaccacgcccactgaagggcgactgcacccaaatgctggtaacactgtggtccacaagg

SEQ ID NO: 284 - DNA, 91 bp, synthetic construct
  source 1..91: mol_type = other DNA
acgcacactggacgcgcaaaaaaaggtgcagtcgcccttcagtgggcgtggtcggtgcaacccttgtggaccacagtgttaccagcatttg

SEQ ID NO: 285 - DNA, 90 bp, synthetic construct
  source 1..90: mol_type = other DNA
cttgtggaaaggacgaaacaccggttgcaccgaccacgcccactgaagggcgactgcaccgtgcagctcctcagtcacaatcagggaagc

SEQ ID NO: 286 - DNA, 91 bp, synthetic construct
  source 1..91: mol_type = other DNA
acgcacactggacgcgcaaaaaaaggtgcagtcgcccttcagtgggcgtggtcggtgcaacgcttccctgattgtgactgaggagctgcac

SEQ ID NO: 287 - DNA, 86 bp, synthetic construct
  source 1..86: mol_type = other DNA
cttgtggaaaggacgaaacaccggtcgctccccattcggggagcgtggattgaaatcaaatgctggtaacactgtggtccacaagg

SEQ ID NO: 288 - DNA, 87 bp, synthetic construct
  source 1..87: mol_type = other DNA
acgcacactggacgcgcaaaaaaaatttcaatccacgctccccgaatggggagcgacccttgtggaccacagtgttaccagcatttg

SEQ ID NO: 289 - DNA, 86 bp, synthetic construct
  source 1..86: mol_type = other DNA
cttgtggaaaggacgaaacaccggtcgctccccattcggggagcgtggattgaaatgtgcagctcctcagtcacaatcagggaagc

SEQ ID NO: 290 - DNA, 87 bp, synthetic construct
  source 1..87: mol_type = other DNA
acgcacactggacgcgcaaaaaaaatttcaatccacgctccccgaatggggagcgacgcttccctgattgtgactgaggagctgcac

SEQ ID NO: 291 - DNA, 89 bp, synthetic construct
  source 1..89: mol_type = other DNA
cttgtggaaaggacgaaacaccggttgtagaagccctcagtttgagagggtataacaaccaaatgctggtaacactgtggtccacaagg

SEQ ID NO: 292 - DNA, 90 bp, synthetic construct
  source 1..90: mol_type = other DNA
acgcacactggacgcgcaaaaaaagttgttataccctctcaaactgagggcttctacaacccttgtggaccacagtgttaccagcatttg

SEQ ID NO: 293 - DNA, 89 bp, synthetic construct
  source 1..89: mol_type = other DNA
cttgtggaaaggacgaaacaccggttgtagaagccctcagtttgagagggtataacaacgtgcagctcctcagtcacaatcagggaagc

SEQ ID NO: 294 - DNA, 90 bp, synthetic construct
  source 1..90: mol_type = other DNA
acgcacactggacgcgcaaaaaaagttgttataccctctcaaactgagggcttctacaacgcttccctgattgtgactgaggagctgcac

SEQ ID NO: 295 - DNA, 89 bp, synthetic construct
  source 1..89: mol_type = other DNA
cttgtggaaaggacgaaacaccggttctagatataagtcagtttaagtacatagaaccccaaatgctggtaacactgtggtccacaagg

SEQ ID NO: 296 - DNA, 90 bp, synthetic construct
  source 1..90: mol_type = other DNA
acgcacactggacgcgcaaaaaaagggttctatgtacttaaactgacttatatctagaacccttgtggaccacagtgttaccagcatttg

SEQ ID NO: 297 - DNA, 89 bp, synthetic construct
  source 1..89: mol_type = other DNA
cttgtggaaaggacgaaacaccggttctagatataagtcagtttaagtacatagaacccgtgcagctcctcagtcacaatcagggaagc

SEQ ID NO: 298 - DNA, 90 bp, synthetic construct
  source 1..90: mol_type = other DNA
acgcacactggacgcgcaaaaaaagggttctatgtacttaaactgacttatatctagaacgcttccctgattgtgactgaggagctgcac

SEQ ID NO: 299 - DNA, 89 bp, synthetic construct
  source 1..89: mol_type = other DNA
cttgtggaaaggacgaaacaccggtggcgacgggtgaggaggccggatcgggttggaggcaaatgctggtaacactgtggtccacaagg

SEQ ID NO: 300 - DNA, 90 bp, synthetic construct
  source 1..90: mol_type = other DNA
acgcacactggacgcgcaaaaaaacctccaacccgatccggcctcctcacccgtcgccacccttgtggaccacagtgttaccagcatttg

SEQ ID NO: 301 - DNA, 89 bp, synthetic construct
  source 1..89: mol_type = other DNA
cttgtggaaaggacgaaacaccggtggcgacgggtgaggaggccggatcgggttggagggtgcagctcctcagtcacaatcagggaagc

SEQ ID NO: 302 - DNA, 90 bp, synthetic construct
  source 1..90: mol_type = other DNA
acgcacactggacgcgcaaaaaaacctccaacccgatccggcctcctcacccgtcgccacgcttccctgattgtgactgaggagctgcac

SEQ ID NO: 303 - DNA, 83 bp, synthetic construct
  source 1..83: mol_type = other DNA
cttgtggaaaggacgaaacaccggttattatcgtccctataaggggttgaaaccaaatgctggtaacactgtggtccacaagg

SEQ ID NO: 304 - DNA, 84 bp, synthetic construct
  source 1..84: mol_type = other DNA
acgcacactggacgcgcaaaaaaagtttcaaccccttatagggacgataataacccttgtggaccacagtgttaccagcatttg

SEQ ID NO: 305 - DNA, 83 bp, synthetic construct
  source 1..83: mol_type = other DNA
cttgtggaaaggacgaaacaccggttattatcgtccctataaggggttgaaacgtgcagctcctcagtcacaatcagggaagc

SEQ ID NO: 306 - DNA, 84 bp, synthetic construct
  source 1..84: mol_type = other DNA
acgcacactggacgcgcaaaaaaagtttcaaccccttatagggacgataataacgcttccctgattgtgactgaggagctgcac

SEQ ID NO: 307 - DNA, 89 bp, synthetic construct
  source 1..89: mol_type = other DNA
cttgtggaaaggacgaaacaccggttgtagttccctttcactttgggatcattcacacccaaatgctggtaacactgtggtccacaagg

SEQ ID NO: 308 - DNA, 90 bp, synthetic construct
  source 1..90: mol_type = other DNA
acgcacactggacgcgcaaaaaaaggtgtgaatgatcccaaagtgaaagggaactacaacccttgtggaccacagtgttaccagcatttg

SEQ ID NO: 309 - DNA, 89 bp, synthetic construct
  source 1..89: mol_type = other DNA
cttgtggaaaggacgaaacaccggttgtagttccctttcactttgggatcattcacaccgtgcagctcctcagtcacaatcagggaagc

SEQ ID NO: 310 - DNA, 90 bp, synthetic construct
  source 1..90: mol_type = other DNA
acgcacactggacgcgcaaaaaaaggtgtgaatgatcccaaagtgaaagggaactacaacgcttccctgattgtgactgaggagctgcac

SEQ ID NO: 311 - DNA, 90 bp, synthetic construct
  source 1..90: mol_type = other DNA
cttgtggaaaggacgaaacaccggcccccaacaaaccatcagccgaaaggcgattgagaccaaatgctggtaacactgtggtccacaagg

SEQ ID NO: 312 - DNA, 91 bp, synthetic construct
  source 1..91: mol_type = other DNA
acgcacactggacgcgcaaaaaaagtctcaatcgcctttcggctgatggtttgttgggggcccttgtggaccacagtgttaccagcatttg

SEQ ID NO: 313 - DNA, 90 bp, synthetic construct
  source 1..90: mol_type = other DNA
cttgtggaaaggacgaaacaccggcccccaacaaaccatcagccgaaaggcgattgagacgtgcagctcctcagtcacaatcagggaagc

SEQ ID NO: 314 - DNA, 91 bp, synthetic construct
  source 1..91: mol_type = other DNA
acgcacactggacgcgcaaaaaaagtctcaatcgcctttcggctgatggtttgttgggggcgcttccctgattgtgactgaggagctgcac

SEQ ID NO: 315 - DNA, 89 bp, synthetic construct
  source 1..89: mol_type = other DNA
cttgtggaaaggacgaaacaccggttggagatcacccccaaatcgagggggactgcacccaaatgctggtaacactgtggtccacaagg

SEQ ID NO: 316 - DNA, 90 bp, synthetic construct
  source 1..90: mol_type = other DNA
acgcacactggacgcgcaaaaaaaggtgcagtccccctcgatttgggggtgatctccaacccttgtggaccacagtgttaccagcatttg

SEQ ID NO: 317 - DNA, 89 bp, synthetic construct
  source 1..89: mol_type = other DNA
cttgtggaaaggacgaaacaccggttggagatcacccccaaatcgagggggactgcaccgtgcagctcctcagtcacaatcagggaagc

SEQ ID NO: 318 - DNA, 90 bp, synthetic construct
  source 1..90: mol_type = other DNA
acgcacactggacgcgcaaaaaaaggtgcagtccccctcgatttgggggtgatctccaacgcttccctgattgtgactgaggagctgcac

SEQ ID NO: 319 - DNA, 86 bp, synthetic construct
  source 1..86: mol_type = other DNA
cttgtggaaaggacgaaacaccggtcgaggctcgcgagagccttgtggattgaaatcaaatgctggtaacactgtggtccacaagg

SEQ ID NO: 320 - DNA, 87 bp, synthetic construct
  source 1..87: mol_type = other DNA
acgcacactggacgcgcaaaaaaaatttcaatccacaaggctctcgcgagcctcgacccttgtggaccacagtgttaccagcatttg

SEQ ID NO: 321 - DNA, 86 bp, synthetic construct
  source 1..86: mol_type = other DNA
cttgtggaaaggacgaaacaccggtcgaggctcgcgagagccttgtggattgaaatgtgcagctcctcagtcacaatcagggaagc

SEQ ID NO: 322 - DNA, 87 bp, synthetic construct
  source 1..87: mol_type = other DNA
acgcacactggacgcgcaaaaaaaatttcaatccacaaggctctcgcgagcctcgacgcttccctgattgtgactgaggagctgcac

SEQ ID NO: 323 - DNA, 86 bp, synthetic construct
  source 1..86: mol_type = other DNA
cttgtggaaaggacgaaacaccggtcgccttccccccggaaggcgtggattgaaaccaaatgctggtaacactgtggtccacaagg

SEQ ID NO: 324 - DNA, 87 bp, synthetic construct
  source 1..87: mol_type = other DNA
acgcacactggacgcgcaaaaaaagtttcaatccacgccttccggggggaaggcgacccttgtggaccacagtgttaccagcatttg

SEQ ID NO: 325 - DNA, 86 bp, synthetic construct
  source 1..86: mol_type = other DNA
cttgtggaaaggacgaaacaccggtcgccttccccccggaaggcgtggattgaaacgtgcagctcctcagtcacaatcagggaagc

SEQ ID NO: 326 - DNA, 87 bp, synthetic construct
  source 1..87: mol_type = other DNA
acgcacactggacgcgcaaaaaaagtttcaatccacgccttccggggggaaggcgacgcttccctgattgtgactgaggagctgcac

SEQ ID NO: 327 - DNA, 82 bp, synthetic construct
  source 1..82: mol_type = other DNA
cttgtggaaaggacgaaacaccggtttgccccgcatgtgcggggatgatccgcaaatgctggtaacactgtggtccacaagg

SEQ ID NO: 328 - DNA, 83 bp, synthetic construct
  source 1..83: mol_type = other DNA
acgcacactggacgcgcaaaaaaacggatcatccccgcacatgcggggcaaacccttgtggaccacagtgttaccagcatttg

SEQ ID NO: 329 - DNA, 82 bp, synthetic construct
  source 1..82: mol_type = other DNA
cttgtggaaaggacgaaacaccggtttgccccgcatgtgcggggatgatccggtgcagctcctcagtcacaatcagggaagc

SEQ ID NO: 330 - DNA, 83 bp, synthetic construct
  source 1..83: mol_type = other DNA
acgcacactggacgcgcaaaaaaacggatcatccccgcacatgcggggcaaacgcttccctgattgtgactgaggagctgcac

SEQ ID NO: 331 - DNA, 76 bp, synthetic construct
  source 1..76: mol_type = other DNA
cttgtggaaaggacgaaacaccgctccttctgctcaggcgtggcttcaaatgctggtaacactgtggtccacaagg

SEQ ID NO: 332 - DNA, 77 bp, synthetic construct
  source 1..77: mol_type = other DNA
acgcacactggacgcgcaaaaaaaaagccacgcctgagcagaaggagccttgtggaccacagtgttaccagcatttg

SEQ ID NO: 333 - DNA, 76 bp, synthetic construct
  source 1..76: mol_type = other DNA
cttgtggaaaggacgaaacaccgctccttctgctcaggcgtggcttgtgcagctcctcagtcacaatcagggaagc

SEQ ID NO: 334 - DNA, 77 bp, synthetic construct
  source 1..77: mol_type = other DNA
acgcacactggacgcgcaaaaaaaaagccacgcctgagcagaaggaggcttccctgattgtgactgaggagctgcac

SEQ ID NO: 335 - DNA, 76 bp, synthetic construct
  source 1..76: mol_type = other DNA
cttgtggaaaggacgaaacaccgaagccacgcctgagcagaaggagcaaatgctggtaacactgtggtccacaagg

SEQ ID NO: 336 - DNA, 77 bp, synthetic construct
  source 1..77: mol_type = other DNA
acgcacactggacgcgcaaaaaaactccttctgctcaggcgtggcttccttgtggaccacagtgttaccagcatttg

SEQ ID NO: 337 - DNA, 76 bp, synthetic construct
  source 1..76: mol_type = other DNA
cttgtggaaaggacgaaacaccgaagccacgcctgagcagaaggaggtgcagctcctcagtcacaatcagggaagc

SEQ ID NO: 338 - DNA, 77 bp, synthetic construct
  source 1..77: mol_type = other DNA
acgcacactggacgcgcaaaaaaactccttctgctcaggcgtggcttgcttccctgattgtgactgaggagctgcac

SEQ ID NO: 339 - DNA, 76 bp, synthetic construct
  source 1..76: mol_type = other DNA
cttgtggaaaggacgaaacaccgaagccacgcctgagcagaaggaggcggtgggctcggtcctgcgcttgcaggtc

SEQ ID NO: 340 - DNA, 77 bp, synthetic construct
  source 1..77: mol_type = other DNA
acgcacactggacgcgcaaaaaaactccttctgctcaggcgtggcttgacctgcaagcgcaggaccgagcccaccgc

SEQ ID NO: 341 - DNA, 76 bp, synthetic construct
  source 1..76: mol_type = other DNA
cttgtggaaaggacgaaacaccgaagccacgcctgagcagaaggagtccgagtccttcacccgtttgatctgctcc

SEQ ID NO: 342 - DNA, 77 bp, synthetic construct
  source 1..77: mol_type = other DNA
acgcacactggacgcgcaaaaaaactccttctgctcaggcgtggcttggagcagatcaaacgggtgaaggactcgga

SEQ ID NO: 343 - DNA, 76 bp, synthetic construct
  source 1..76: mol_type = other DNA
cttgtggaaaggacgaaacaccgaagccacgcctgagcagaaggaggtttctggcagttctcctctcctgcacccc

SEQ ID NO: 344 - DNA, 77 bp, synthetic construct
  source 1..77: mol_type = other DNA
acgcacactggacgcgcaaaaaaactccttctgctcaggcgtggcttggggtgcaggagaggagaactgccagaaac

SEQ ID NO: 345 - DNA, 76 bp, synthetic construct
  source 1..76: mol_type = other DNA
cttgtggaaaggacgaaacaccgaagccacgcctgagcagaaggagcggcctgtggcatccgcccaaacctgatgg

SEQ ID NO: 346 - DNA, 77 bp, synthetic construct
  source 1..77: mol_type = other DNA
acgcacactggacgcgcaaaaaaactccttctgctcaggcgtggcttccatcaggtttgggcggatgccacaggccg

SEQ ID NO: 347 - DNA, 76 bp, synthetic construct
  source 1..76: mol_type = other DNA
cttgtggaaaggacgaaacaccgaagccacgcctgagcagaaggagagtttctaaacagctccacgattctctcct

SEQ ID NO: 348 - DNA, 77 bp, synthetic construct
  source 1..77: mol_type = other DNA
acgcacactggacgcgcaaaaaaactccttctgctcaggcgtggcttaggagagaatcgtggagctgtttagaaact

SEQ ID NO: 349 - DNA, 76 bp, synthetic construct
  source 1..76: mol_type = other DNA
cttgtggaaaggacgaaacaccgaagccacgcctgagcagaaggagtctgacaccctgaataattcacaccaggtc

SEQ ID NO: 350 - DNA, 77 bp, synthetic construct
  source 1..77: mol_type = other DNA
acgcacactggacgcgcaaaaaaactccttctgctcaggcgtggcttgacctggtgtgaattattcagggtgtcaga

SEQ ID NO: 351 - DNA, 76 bp, synthetic construct
  source 1..76: mol_type = other DNA
cttgtggaaaggacgaaacaccgaagccacgcctgagcagaaggagagcccatgatgtacccttcgttccaaaggg

SEQ ID NO: 352 - DNA, 77 bp, synthetic construct
  source 1..77: mol_type = other DNA
acgcacactggacgcgcaaaaaaactccttctgctcaggcgtggcttccctttggaacgaagggtacatcatgggct

SEQ ID NO: 353 - DNA, 76 bp, synthetic construct
  source 1..76: mol_type = other DNA
cttgtggaaaggacgaaacaccgaagccacgcctgagcagaaggagatgaggactctaaacattgaggcttcagca

SEQ ID NO: 354 - DNA, 77 bp, synthetic construct
  source 1..77: mol_type = other DNA
acgcacactggacgcgcaaaaaaactccttctgctcaggcgtggctttgctgaagcctcaatgtttagagtcctcat

SEQ ID NO: 355 - DNA, 76 bp, synthetic construct
  source 1..76: mol_type = other DNA
cttgtggaaaggacgaaacaccgaagccacgcctgagcagaaggaggacagaggtcagggtcacactctcggacag

SEQ ID NO: 356 - DNA, 77 bp, synthetic construct
  source 1..77: mol_type = other DNA
acgcacactggacgcgcaaaaaaactccttctgctcaggcgtggcttagttggtgaatgcccttggtcaatataatg

SEQ ID NO: 357 - DNA, 76 bp, synthetic construct
  source 1..76: mol_type = other DNA
cttgtggaaaggacgaaacaccgaagccacgcctgagcagaaggagaaatcccccagcctgccacgtcagatggtg

SEQ ID NO: 358 - DNA, 77 bp, synthetic construct
  source 1..77: mol_type = other DNA
acgcacactggacgcgcaaaaaaactccttctgctcaggcgtggcttcaccatctgacgtggcaggctgggggattt

SEQ ID NO: 359 - DNA, 76 bp, synthetic construct
  source 1..76: mol_type = other DNA
cttgtggaaaggacgaaacaccgaagccacgcctgagcagaaggagctgtgttgagggcaatgaggacataaccag

SEQ ID NO: 360 - DNA, 77 bp, synthetic construct
  source 1..77: mol_type = other DNA
acgcacactggacgcgcaaaaaaactccttctgctcaggcgtggcttctggttatgtcctcattgccctcaacacag

SEQ ID NO: 361 - DNA, 76 bp, synthetic construct
  source 1..76: mol_type = other DNA
cttgtggaaaggacgaaacaccgaagccacgcctgagcagaaggagtgtggcgccttcgcatgaagaggccgatcc

SEQ ID NO: 362 - DNA, 77 bp, synthetic construct
  source 1..77: mol_type = other DNA
acgcacactggacgcgcaaaaaaactccttctgctcaggcgtggcttggatcggcctcttcatgcgaaggcgccaca

SEQ ID NO: 363 - DNA, 76 bp, synthetic construct
  source 1..76: mol_type = other DNA
cttgtggaaaggacgaaacaccgaagccacgcctgagcagaaggagtgagctgcacggtggaggtgaggcagatgc

SEQ ID NO: 364 - DNA, 77 bp, synthetic construct
  source 1..77: mol_type = other DNA
acgcacactggacgcgcaaaaaaactccttctgctcaggcgtggcttgcatctgcctcacctccaccgtgcagctca

SEQ ID NO: 365 - DNA, 76 bp, synthetic construct
  source 1..76: mol_type = other DNA
cttgtggaaaggacgaaacaccgaagccacgcctgagcagaaggagcagtgcgtgcagccaggtcacacttgttcc

SEQ ID NO: 366 - DNA, 77 bp, synthetic construct
  source 1..77: mol_type = other DNA
acgcacactggacgcgcaaaaaaactccttctgctcaggcgtggcttggaacaagtgtgacctggctgcacgcactg

SEQ ID NO: 367 - DNA, 76 bp, synthetic construct
  source 1..76: mol_type = other DNA
cttgtggaaaggacgaaacaccgaagccacgcctgagcagaaggagcttgtgaacactggggtcgtagtcaccata

SEQ ID NO: 368 - DNA, 77 bp, synthetic construct
  source 1..77: mol_type = other DNA
acgcacactggacgcgcaaaaaaactccttctgctcaggcgtggctttatggtgactacgaccccagtgttcacaag

SEQ ID NO: 369 - DNA, 76 bp, synthetic construct
  source 1..76: mol_type = other DNA
cttgtggaaaggacgaaacaccgaagccacgcctgagcagaaggagtagtcaccatacttggcctggacggcgtaa

SEQ ID NO: 370 - DNA, 77 bp, synthetic construct
  source 1..77: mol_type = other DNA
acgcacactggacgcgcaaaaaaactccttctgctcaggcgtggcttttacgccgtccaggccaagtatggtgacta

SEQ ID NO: 371 - DNA, 76 bp, synthetic construct
  source 1..76: mol_type = other DNA
cttgtggaaaggacgaaacaccgaagccacgcctgagcagaaggagacggcgtaagaagccaggagcacagaagcc

SEQ ID NO: 372 - DNA, 77 bp, synthetic construct
  source 1..77: mol_type = other DNA
acgcacactggacgcgcaaaaaaactccttctgctcaggcgtggcttggcttctgtgctcctggcttcttacgccgt

SEQ ID NO: 373 - DNA, 76 bp, synthetic construct
  source 1..76: mol_type = other DNA
cttgtggaaaggacgaaacaccgaagccacgcctgagcagaaggaggcctcggtgctctgcgtaccaagcagtaat

SEQ ID NO: 374 - DNA, 77 bp, synthetic construct
  source 1..77: mol_type = other DNA
acgcacactggacgcgcaaaaaaactccttctgctcaggcgtggcttattactgcttggtacgcagagcaccgaggc

SEQ ID NO: 375 - DNA, 76 bp, synthetic construct; identical to SEQ ID NO: 339
  source 1..76: mol_type = other DNA

SEQ ID NO: 376 - DNA, 77 bp, synthetic construct; identical to SEQ ID NO: 340
  source 1..77: mol_type = other DNA

SEQ ID NO: 377 - DNA, 76 bp, synthetic construct
  source 1..76: mol_type = other DNA
cttgtggaaaggacgaaacaccgaagccacgcctgagcagaaggaggcttgcaggtcctggtgaggattccagtcg

SEQ ID NO: 378 - DNA, 77 bp, synthetic construct
  source 1..77: mol_type = other DNA
acgcacactggacgcgcaaaaaaactccttctgctcaggcgtggcttcgactggaatcctcaccaggacctgcaagc

SEQ ID NO: 379 - DNA, 76 bp, synthetic construct
  source 1..76: mol_type = other DNA
cttgtggaaaggacgaaacaccgaagccacgcctgagcagaaggagctgtcaaaaatgatcacagtgtctgccgac

SEQ ID NO: 380 - DNA, 77 bp, synthetic construct
  source 1..77: mol_type = other DNA
acgcacactggacgcgcaaaaaaactccttctgctcaggcgtggcttgtcggcagacactgtgatcatttttgacag

SEQ ID NO: 381 - DNA, 76 bp, synthetic construct
  source 1..76: mol_type = other DNA
cttgtggaaaggacgaaacaccgaagccacgcctgagcagaaggagctgcagctaggatcttctcctccacgctgt

SEQ ID NO: 382 - DNA, 77 bp, synthetic construct
  source 1..77: mol_type = other DNA
acgcacactggacgcgcaaaaaaactccttctgctcaggcgtggcttacagcgtggaggagaagatcctagctgcag

SEQ ID NO: 383 - DNA, 76 bp, synthetic construct
  source 1..76: mol_type = other DNA
cttgtggaaaggacgaaacaccgaagccacgcctgagcagaaggagcattatgagacatccccactgcaaggcatt

SEQ ID NO: 384 - DNA, 77 bp, synthetic construct
  source 1..77: mol_type = other DNA
acgcacactggacgcgcaaaaaaactccttctgctcaggcgtggcttaatgccttgcagtggggatgtctcataatg

SEQ ID NO: 385 - DNA, 76 bp, synthetic construct; identical to SEQ ID NO: 345
  source 1..76: mol_type = other DNA

SEQ ID NO: 386 - DNA, 77 bp, synthetic construct; identical to SEQ ID NO: 346
  source 1..77: mol_type = other DNA

SEQ ID NO: 387 - DNA, 76 bp, synthetic construct
  source 1..76: mol_type = other DNA
cttgtggaaaggacgaaacaccgaagccacgcctgagcagaaggagatatcactggagatctccgccaacagcttc

SEQ ID NO: 388 - DNA, 77 bp, synthetic construct
  source 1..77: mol_type = other DNA
acgcacactggacgcgcaaaaaaactccttctgctcaggcgtggcttgaagctgttggcggagatctccagtgatat

SEQ ID NO: 389 - DNA, 76 bp, synthetic construct
  source 1..76: mol_type = other DNA
cttgtggaaaggacgaaacaccgaagccacgcctgagcagaaggagtggatccgacagttaagatcacatctgtca

SEQ ID NO: 390 - DNA, 77 bp, synthetic construct
  source 1..77: mol_type = other DNA
acgcacactggacgcgcaaaaaaactccttctgctcaggcgtggctttgacagatgtgatcttaactgtcggatcca

SEQ ID NO: 391 - DNA, 76 bp, synthetic construct
  source 1..76: mol_type = other DNA
cttgtggaaaggacgaaacaccgaagccacgcctgagcagaaggagcaccagctctctgactgtacccccagagac

SEQ ID NO: 392 - DNA, 77 bp, synthetic construct
  source 1..77: mol_type = other DNA
acgcacactggacgcgcaaaaaaactccttctgctcaggcgtggcttgtctctgggggtacagtcagagagctggtg

SEQ ID NO: 393 - DNA, 76 bp, synthetic construct
  source 1..76: mol_type = other DNA
cttgtggaaaggacgaaacaccgaagccacgcctgagcagaaggagcccccagagacctcatagttgtccataagt

SEQ ID NO: 394 - DNA, 77 bp, synthetic construct
  source 1..77: mol_type = other DNA
acgcacactggacgcgcaaaaaaactccttctgctcaggcgtggcttacttatggacaactatgaggtctctggggg

SEQ ID NO: 395 - DNA, 76 bp, synthetic construct
  source 1..76: mol_type = other DNA
cttgtggaaaggacgaaacaccgaagccacgcctgagcagaaggagaaggagcaggactcagccggaaggcattat

SEQ ID NO: 396 - DNA, 77 bp, synthetic construct
  source 1..77: mol_type = other DNA
acgcacactggacgcgcaaaaaaactccttctgctcaggcgtggcttataatgccttccggctgagtcctgctcctt

SEQ ID NO: 397 - DNA, 76 bp, synthetic construct
  source 1..76: mol_type = other DNA
cttgtggaaaggacgaaacaccgaagccacgcctgagcagaaggagatcacttcaattgcttcggtgtagcccatt

SEQ ID NO: 398 - DNA, 77 bp, synthetic construct
  source 1..77: mol_type = other DNA
acgcacactggacgcgcaaaaaaactccttctgctcaggcgtggcttaatgggctacaccgaagcaattgaagtgat

SEQ ID NO: 399 - DNA, 76 bp, synthetic construct
  source 1..76: mol_type = other DNA
cttgtggaaaggacgaaacaccgaagccacgcctgagcagaaggagggctgattcgctgtgacttcgaattgcatc

SEQ ID NO: 400 - DNA, 77 bp, synthetic construct
  source 1..77: mol_type = other DNA
acgcacactggacgcgcaaaaaaactccttctgctcaggcgtggcttgatgcaattcgaagtcacagcgaatcagcc

SEQ ID NO: 401 - DNA, 76 bp, synthetic construct
  source 1..76: mol_type = other DNA
cttgtggaaaggacgaaacaccgaagccacgcctgagcagaaggagcgaattgcatcctcaatcatcctgctgtcc

SEQ ID NO: 402 - DNA, 77 bp, synthetic construct
  source 1..77: mol_type = other DNA
acgcacactggacgcgcaaaaaaactccttctgctcaggcgtggcttggacagcaggatgattgaggatgcaattcg

SEQ ID NO: 403 - DNA, 76 bp, synthetic construct
  source 1..76: mol_type = other DNA
cttgtggaaaggacgaaacaccgaagccacgcctgagcagaaggagtgctgaccatgtggacattaggtgtggatg

SEQ ID NO: 404 - DNA, 77 bp, synthetic construct
  source 1..77: mol_type = other DNA
acgcacactggacgcgcaaaaaaactccttctgctcaggcgtggcttcatccacacctaatgtccacatggtcagca

SEQ ID NO: 405 - DNA, 76 bp, synthetic construct
  source 1..76: mol_type = other DNA
cttgtggaaaggacgaaacaccgaagccacgcctgagcagaaggaggctcagattgttggggctactggacagggc

SEQ ID NO: 406 - DNA, 77 bp, synthetic construct
  source 1..77: mol_type = other DNA
acgcacactggacgcgcaaaaaaactccttctgctcaggcgtggcttgccctgtccagtagccccaacaatctgagc

SEQ ID NO: 407 - DNA, 76 bp, synthetic construct
  source 1..76: mol_type = other DNA
cttgtggaaaggacgaaacaccgaagccacgcctgagcagaaggagtgatacacctcggtctcaaaggtgatcagg

SEQ ID NO: 408 - DNA, 77 bp, synthetic construct
  source 1..77: mol_type = other DNA
acgcacactggacgcgcaaaaaaactccttctgctcaggcgtggcttcctgatcacctttgagaccgaggtgtatca

SEQ ID NO: 409 - DNA, 76 bp, synthetic construct
  source 1..76: mol_type = other DNA
cttgtggaaaggacgaaacaccgaagccacgcctgagcagaaggagaattctgcagagaggctgccgttgttggat

SEQ ID NO: 410 - DNA, 77 bp, synthetic construct
  source 1..77: mol_type = other DNA
acgcacactggacgcgcaaaaaaactccttctgctcaggcgtggcttatccaacaacggcagcctctctgcagaatt

SEQ ID NO: 411 - DNA, 76 bp, synthetic construct
  source 1..76: mol_type = other DNA
cttgtggaaaggacgaaacaccgaagccacgcctgagcagaaggaggagatcaccacaactggcaaggagtgggtc

SEQ ID NO: 412 - DNA, 77 bp, synthetic construct
  source 1..77: mol_type = other DNA
acgcacactggacgcgcaaaaaaactccttctgctcaggcgtggcttgacccactccttgccagttgtggtgatctc

SEQ ID NO: 413 - DNA, 76 bp, synthetic construct
  source 1..76: mol_type = other DNA
cttgtggaaaggacgaaacaccgaagccacgcctgagcagaaggagcatctgacagatgttggagatcaccacaac

SEQ ID NO: 414 - DNA, 77 bp, synthetic construct
  source 1..77: mol_type = other DNA
acgcacactggacgcgcaaaaaaactccttctgctcaggcgtggcttgttgtggtgatctccaacatctgtcagatg

SEQ ID NO: 415 - DNA, 76 bp, synthetic construct
  source 1..76: mol_type = other DNA
cttgtggaaaggacgaaacaccgaagccacgcctgagcagaaggagacgcccaggcatttggcatctgacagatgt

SEQ ID NO: 416 - DNA, 77 bp, synthetic construct
  source 1..77: mol_type = other DNA
acgcacactggacgcgcaaaaaaactccttctgctcaggcgtggcttacatctgtcagatgccaaatgcctgggcgt

SEQ ID NO: 417 - DNA, 76 bp, synthetic construct
  source 1..76: mol_type = other DNA
cttgtggaaaggacgaaacaccgaagccacgcctgagcagaaggagatcacaattggctcggcccccattcccaca

SEQ ID NO: 418 - DNA, 77 bp, synthetic construct
  source 1..77: mol_type = other DNA
acgcacactggacgcgcaaaaaaactccttctgctcaggcgtggctttgtgggaatgggggccgagccaattgtgat

SEQ ID NO: 419 - DNA, 76 bp, synthetic construct
  source 1..76: mol_type = other DNA
cttgtggaaaggacgaaacaccgaagccacgcctgagcagaaggagtcctcgaagttcatcacgcgctcccacttg

SEQ ID NO: 420 - DNA, 77 bp, synthetic construct
  source 1..77: mol_type = other DNA
acgcacactggacgcgcaaaaaaactccttctgctcaggcgtggcttcaagtgggagcgcgtgatgaacttcgagga

SEQ ID NO: 421 - DNA, 76 bp, synthetic construct
  source 1..76: mol_type = other DNA
cttgtggaaaggacgaaacaccgaagccacgcctgagcagaaggagtgcttcacgtaggccttggagccgtacatg

SEQ ID NO: 422 - DNA, 77 bp, synthetic construct
  source 1..77: mol_type = other DNA
acgcacactggacgcgcaaaaaaactccttctgctcaggcgtggcttcatgtacggctccaaggcctacgtgaagca

SEQ ID NO: 423 - DNA, 76 bp, synthetic construct
  source 1..76: mol_type = other DNA
cttgtggaaaggacgaaacaccgaagccacgcctgagcagaaggagaagttcatcacgcgctcccacttgaagccc

SEQ ID NO: 424 - DNA, 77 bp, synthetic construct
  source 1..77: mol_type = other DNA
acgcacactggacgcgcaaaaaaactccttctgctcaggcgtggcttgggcttcaagtgggagcgcgtgatgaactt

SEQ ID NO: 425 - DNA, 76 bp, synthetic construct
  source 1..76: mol_type = other DNA
cttgtggaaaggacgaaacaccgaagccacgcctgagcagaaggaggagccgtacatgaactgaggggacaggatg

SEQ ID NO: 426 - DNA, 95 bp, synthetic construct
  source 1..95: mol_type = other DNA
acgcacactggacgcgcaaaaaaactccttctgctcaggcgtggcttcatcctgtcccctcagttcatgtacggctccaaggcctacgtgaagca

SEQ ID NO: 427 - DNA, 90 bp, synthetic construct
  source 1..90: mol_type = other DNA
cttgtggaaaggacgaaacaccgcgtttccacggcatcacagccgtggccgaattgaagccaaatgctggtaacactgtggtccacaagg

SEQ ID NO: 428 - DNA, 91 bp, synthetic construct
  source 1..91: mol_type = other DNA
acgcacactggacgcgcaaaaaaagcttcaattcggccacggctgtgatgccgtggaaacgccttgtggaccacagtgttaccagcatttg

SEQ ID NO: 429 - DNA, 90 bp, synthetic construct
  source 1..90: mol_type = other DNA
cttgtggaaaggacgaaacaccgcgtttccacggcatcacagccgtggccgaattgaagcgtgcagctcctcagtcacaatcagggaagc

SEQ ID NO: 430 - DNA, 91 bp, synthetic construct
  source 1..91: mol_type = other DNA
acgcacactggacgcgcaaaaaaagcttcaattcggccacggctgtgatgccgtggaaacggcttccctgattgtgactgaggagctgcac

SEQ ID NO: 431 - DNA, 90 bp, synthetic construct
  source 1..90: mol_type = other DNA
cttgtggaaaggacgaaacaccggtaagaatcaaataatcccgatacgcgggattaagaccaaatgctggtaacactgtggtccacaagg

SEQ ID NO: 432 - DNA, 91 bp, synthetic construct
  source 1..91: mol_type = other DNA
acgcacactggacgcgcaaaaaaagtcttaatcccgcgtatcgggattatttgattcttacccttgtggaccacagtgttaccagcatttg

SEQ ID NO: 433 - DNA, 90 bp, synthetic construct
  source 1..90: mol_type = other DNA
cttgtggaaaggacgaaacaccggtaagaatcaaataatcccgatacgcgggattaagacgtgcagctcctcagtcacaatcagggaagc

SEQ ID NO: 434 - DNA, 91 bp, synthetic construct
  source 1..91: mol_type = other DNA
acgcacactggacgcgcaaaaaaagtcttaatcccgcgtatcgggattatttgattcttacgcttccctgattgtgactgaggagctgcac

SEQ ID NO: 435 - DNA, 85 bp, synthetic construct
  source 1..85: mol_type = other DNA
cttgtggaaaggacgaaacaccggctgcattccccgcgcgagaggggattgagaccaaatgctggtaacactgtggtccacaagg

SEQ ID NO: 436 - DNA, 86 bp, synthetic construct
  source 1..86: mol_type = other DNA
acgcacactggacgcgcaaaaaaagtctcaatcccctctcgcgcggggaatgcagcccttgtggaccacagtgttaccagcatttg

SEQ ID NO: 437 - DNA, 85 bp, synthetic construct
  source 1..85: mol_type = other DNA
cttgtggaaaggacgaaacaccggctgcattccccgcgcgagaggggattgagacgtgcagctcctcagtcacaatcagggaagc

SEQ ID NO: 438 - DNA, 86 bp, synthetic construct
  source 1..86: mol_type = other DNA
acgcacactggacgcgcaaaaaaagtctcaatcccctctcgcgcggggaatgcagcgcttccctgattgtgactgaggagctgcac

SEQ ID NO: 439 - DNA, 89 bp, synthetic construct
  source 1..89: mol_type = other DNA
cttgtggaaaggacgaaacaccggttgtgtgtacccttcgaatagagggtagatccaaccaaatgctggtaacactgtggtccacaagg

SEQ ID NO: 440 - DNA, 90 bp, synthetic construct
  source 1..90: mol_type = other DNA
acgcacactggacgcgcaaaaaaagttggatctaccctctattcgaagggtacacacaacccttgtggaccacagtgttaccagcatttg

SEQ ID NO: 441 - DNA, 89 bp, synthetic construct
  source 1..89: mol_type = other DNA
cttgtggaaaggacgaaacaccggttgtgtgtacccttcgaatagagggtagatccaacgtgcagctcctcagtcacaatcagggaagc

SEQ ID NO: 442 - DNA, 90 bp, synthetic construct
  source 1..90: mol_type = other DNA
acgcacactggacgcgcaaaaaaagttggatctaccctctattcgaagggtacacacaacgcttccctgattgtgactgaggagctgcac

SEQ ID NO: 443 - DNA, 84 bp, synthetic construct
  source 1..84: mol_type = other DNA
cttgtggaaaggacgaaacaccggtcgcgccttcgcgggcgcgtgagttgaaaccaaatgctggtaacactgtggtccacaagg

SEQ ID NO: 444 - DNA, 85 bp, synthetic construct
  source 1..85: mol_type = other DNA
acgcacactggacgcgcaaaaaaagtttcaactcacgcgcccgcgaaggcgcgacccttgtggaccacagtgttaccagcatttg

SEQ ID NO: 445 - DNA, 84 bp, synthetic construct
  source 1..84: mol_type = other DNA
cttgtggaaaggacgaaacaccggtcgcgccttcgcgggcgcgtgagttgaaacgtgcagctcctcagtcacaatcagggaagc

SEQ ID NO: 446 - DNA, 85 bp, synthetic construct
  source 1..85: mol_type = other DNA
acgcacactggacgcgcaaaaaaagtttcaactcacgcgcccgcgaaggcgcgacgcttccctgattgtgactgaggagctgcac

SEQ ID NO: 447 - DNA, 81 bp, synthetic construct
  source 1..81: mol_type = other DNA
cttgtggaaaggacgaaacaccgggttcccccgtacacgcggggatagacccaaatgctggtaacactgtggtccacaagg

SEQ ID NO: 448 - DNA, 82 bp, synthetic construct
  source 1..82: mol_type = other DNA
acgcacactggacgcgcaaaaaaaggtctatccccgcgtgtacgggggaaccccttgtggaccacagtgttaccagcatttg

SEQ ID NO: 449 - DNA, 81 bp, synthetic construct
  source 1..81: mol_type = other DNA
cttgtggaaaggacgaaacaccgggttcccccgtacacgcggggatagaccgtgcagctcctcagtcacaatcagggaagc

SEQ ID NO: 450 - DNA, 82 bp, synthetic construct
  source 1..82: mol_type = other DNA
acgcacactggacgcgcaaaaaaaggtctatccccgcgtgtacgggggaaccgcttccctgattgtgactgaggagctgcac

SEQ ID NO: 451 - DNA, 82 bp, synthetic construct
  source 1..82: mol_type = other DNA
cttgtggaaaggacgaaacaccggtgctccccgcacacgcggggatgatccccaaatgctggtaacactgtggtccacaagg

SEQ ID NO: 452 - DNA, 83 bp, synthetic construct
  source 1..83: mol_type = other DNA
acgcacactggacgcgcaaaaaaagggatcatccccgcgtgtgcggggagcacccttgtggaccacagtgttaccagcatttg

SEQ ID NO: 453 - DNA, 82 bp, synthetic construct
  source 1..82: mol_type = other DNA
cttgtggaaaggacgaaacaccggtgctccccgcacacgcggggatgatcccgtgcagctcctcagtcacaatcagggaagc

SEQ ID NO: 454 - DNA, 83 bp, synthetic construct
  source 1..83: mol_type = other DNA
acgcacactggacgcgcaaaaaaagggatcatccccgcgtgtgcggggagcacgcttccctgattgtgactgaggagctgcac

SEQ ID NO: 455 - DNA, 89 bp, synthetic construct
  source 1..89: mol_type = other DNA
cttgtggaaaggacgaaacaccgggtggagacacgcggatttaggggtgtgatgacaggcaaatgctggtaacactgtggtccacaagg

SEQ ID NO: 456 - DNA, 90 bp, synthetic construct
  source 1..90: mol_type = other DNA
acgcacactggacgcgcaaaaaaacctgtcatcacacccctaaatccgcgtgtctccaccccttgtggaccacagtgttaccagcatttg

SEQ ID NO: 457 - DNA, 89 bp, synthetic construct
  source 1..89: mol_type = other DNA
cttgtggaaaggacgaaacaccgggtggagacacgcggatttaggggtgtgatgacagggtgcagctcctcagtcacaatcagggaagc

SEQ ID NO: 458 - DNA, 90 bp, synthetic construct
  source 1..90: mol_type = other DNA
acgcacactggacgcgcaaaaaaacctgtcatcacacccctaaatccgcgtgtctccaccgcttccctgattgtgactgaggagctgcac

SEQ ID NO: 459 - DNA, 88 bp, synthetic construct
  source 1..88: mol_type = other DNA
cttgtggaaaggacgaaacaccgattcctaagctcttacgcttaggacttcattgaggcaaatgctggtaacactgtggtccacaagg

SEQ ID NO: 460 - DNA, 89 bp, synthetic construct
  source 1..89: mol_type = other DNA
acgcacactggacgcgcaaaaaaacctcaatgaagtcctaagcgtaagagcttaggaatccttgtggaccacagtgttaccagcatttg

SEQ ID NO: 461 - DNA, 88 bp, synthetic construct
  source 1..88: mol_type = other DNA
cttgtggaaaggacgaaacaccgattcctaagctcttacgcttaggacttcattgagggtgcagctcctcagtcacaatcagggaagc

SEQ ID NO: 462 - DNA, 89 bp, synthetic construct
  source 1..89: mol_type = other DNA
acgcacactggacgcgcaaaaaaacctcaatgaagtcctaagcgtaagagcttaggaatgcttccctgattgtgactgaggagctgcac

SEQ ID NO: 463 - DNA, 88 bp, synthetic construct
  source 1..88: mol_type = other DNA
cttgtggaaaggacgaaacaccgcctcaatgaagtcctaagcgtaaaagcttaggaatcaaatgctggtaacactgtggtccacaagg

SEQ ID NO: 464 - DNA, 89 bp, synthetic construct
  source 1..89: mol_type = other DNA
acgcacactggacgcgcaaaaaaaattcctaagcttttacgcttaggacttcattgaggccttgtggaccacagtgttaccagcatttg

SEQ ID NO: 465 - DNA, 88 bp, synthetic construct
  source 1..88: mol_type = other DNA
cttgtggaaaggacgaaacaccgcctcaatgaagtcctaagcgtaaaagcttaggaatgtgcagctcctcagtcacaatcagggaagc

SEQ ID NO: 466 - DNA, 89 bp, synthetic construct
  source 1..89: mol_type = other DNA
acgcacactggacgcgcaaaaaaaattcctaagcttttacgcttaggacttcattgagggcttccctgattgtgactgaggagctgcac

SEQ ID NO: 467 - DNA, 89 bp, synthetic construct
  source 1..89: mol_type = other DNA
cttgtggaaaggacgaaacaccgccctcaactattgaaacgtgtttcagtcgtttcaggcaaatgctggtaacactgtggtccacaagg

SEQ ID NO: 468 - DNA, 90 bp, synthetic construct
  source 1..90: mol_type = other DNA
acgcacactggacgcgcaaaaaaacctgaaacgactgaaacacgtttcaatagttgagggccttgtggaccacagtgttaccagcatttg

SEQ ID NO: 469 - DNA, 89 bp, synthetic construct
  source 1..89: mol_type = other DNA
cttgtggaaaggacgaaacaccgccctcaactattgaaacgtgtttcagtcgtttcagggtgcagctcctcagtcacaatcagggaagc

SEQ ID NO: 470 - DNA, 90 bp, synthetic construct
  source 1..90: mol_type = other DNA
acgcacactggacgcgcaaaaaaacctgaaacgactgaaacacgtttcaatagttgaggggcttccctgattgtgactgaggagctgcac

SEQ ID NO: 471 - DNA, 89 bp, synthetic construct
  source 1..89: mol_type = other DNA
cttgtggaaaggacgaaacaccgcctgaaacgactgaaacacgtttcaatagttgagggcaaatgctggtaacactgtggtccacaagg

SEQ ID NO: 472 - DNA, 90 bp, synthetic construct
  source 1..90: mol_type = other DNA
acgcacactggacgcgcaaaaaaaccctcaactattgaaacgtgtttcagtcgtttcaggccttgtggaccacagtgttaccagcatttg

SEQ ID NO: 473 - DNA, 89 bp, synthetic construct
  source 1..89: mol_type = other DNA
cttgtggaaaggacgaaacaccgcctgaaacgactgaaacacgtttcaatagttgaggggtgcagctcctcagtcacaatcagggaagc

SEQ ID NO: 474 - DNA, 90 bp, synthetic construct
  source 1..90: mol_type = other DNA
acgcacactggacgcgcaaaaaaaccctcaactattgaaacgtgtttcagtcgtttcagggcttccctgattgtgactgaggagctgcac

SEQ ID NO: 475 - DNA, 89 bp, synthetic construct
  source 1..89: mol_type = other DNA
cttgtggaaaggacgaaacaccgggtttccgtccccgtgaaggggaagttgtatgaaaccaaatgctggtaacactgtggtccacaagg

SEQ ID NO: 476 - DNA, 90 bp, synthetic construct
  source 1..90: mol_type = other DNA
acgcacactggacgcgcaaaaaaagtttcatacaacttccccttcacggggacggaaaccccttgtggaccacagtgttaccagcatttg

SEQ ID NO: 477 - DNA, 89 bp, synthetic construct
  source 1..89: mol_type = other DNA
cttgtggaaaggacgaaacaccgggtttccgtccccgtgaaggggaagttgtatgaaacgtgcagctcctcagtcacaatcagggaagc

SEQ ID NO: 478 - DNA, 90 bp, synthetic construct
  source 1..90: mol_type = other DNA
acgcacactggacgcgcaaaaaaagtttcatacaacttccccttcacggggacggaaaccgcttccctgattgtgactgaggagctgcac

SEQ ID NO: 479 - DNA, 94 bp, synthetic construct
  source 1..94: mol_type = other DNA
cttgtggaaaggacgaaacaccgttatgtgctcagggccactgcatggtgctgatggaggccaccaaatgctggtaacactgtggtccacaagg

SEQ ID NO: 480 - DNA, 95 bp, synthetic construct
  source 1..95: mol_type = other DNA
acgcacactggacgcgcaaaaaaagtggcctccatcagcaccatgcagtggccctgagcacataaccttgtggaccacagtgttaccagcatttg

SEQ ID NO: 481 - DNA, 94 bp, synthetic construct
  source 1..94: mol_type = other DNA
cttgtggaaaggacgaaacaccgttatgtgctcagggccactgcatggtgctgatggaggccacgtgcagctcctcagtcacaatcagggaagc

SEQ ID NO: 482 - DNA, 95 bp, synthetic construct
  source 1..95: mol_type = other DNA
acgcacactggacgcgcaaaaaaagtggcctccatcagcaccatgcagtggccctgagcacataagcttccctgattgtgactgaggagctgcac

SEQ ID NO: 483 - DNA, 89 bp, synthetic construct
  source 1..89: mol_type = other DNA
cttgtggaaaggacgaaacaccgggtgtcggaaaccgctaattcaggggccgctacaaccaaatgctggtaacactgtggtccacaagg

SEQ ID NO: 484 - DNA, 90 bp, synthetic construct
  source 1..90: mol_type = other DNA
acgcacactggacgcgcaaaaaaagttgtagcggcccctgaattagcggtttccgacaccccttgtggaccacagtgttaccagcatttg

SEQ ID NO: 485 - DNA, 89 bp, synthetic construct
  source 1..89: mol_type = other DNA
cttgtggaaaggacgaaacaccgggtgtcggaaaccgctaattcaggggccgctacaacgtgcagctcctcagtcacaatcagggaagc

SEQ ID NO: 486 - DNA, 90 bp, synthetic construct
  source 1..90: mol_type = other DNA
acgcacactggacgcgcaaaaaaagttgtagcggcccctgaattagcggtttccgacaccgcttccctgattgtgactgaggagctgcac

SEQ ID NO: 487 - DNA, 89 bp, synthetic construct
  source 1..89: mol_type = other DNA
cttgtggaaaggacgaaacaccgggtgtcggaaaccgctaattcaggggccgctacaacgcggtgggctcggtcctgcgcttgcaggtc

SEQ ID NO: 488 - DNA, 90 bp, synthetic construct
  source 1..90: mol_type = other DNA
acgcacactggacgcgcaaaaaaagttgtagcggcccctgaattagcggtttccgacaccgacctgcaagcgcaggaccgagcccaccgc

SEQ ID NO: 489 - DNA, 89 bp, synthetic construct
  source 1..89: mol_type = other DNA
cttgtggaaaggacgaaacaccgggtgtcggaaaccgctaattcaggggccgctacaactccgagtccttcacccgtttgatctgctcc

SEQ ID NO: 490 - DNA, 90 bp, synthetic construct
  source 1..90: mol_type = other DNA
acgcacactggacgcgcaaaaaaagttgtagcggcccctgaattagcggtttccgacaccggagcagatcaaacgggtgaaggactcgga

SEQ ID NO: 491 - DNA, 89 bp, synthetic construct
  source 1..89: mol_type = other DNA
cttgtggaaaggacgaaacaccgggtgtcggaaaccgctaattcaggggccgctacaacgtttctggcagttctcctctcctgcacccc

SEQ ID NO: 492 - DNA, 90 bp, synthetic construct
  source 1..90: mol_type = other DNA
acgcacactggacgcgcaaaaaaagttgtagcggcccctgaattagcggtttccgacaccggggtgcaggagaggagaactgccagaaac

SEQ ID NO: 493 - DNA, 89 bp, synthetic construct
  source 1..89: mol_type = other DNA
cttgtggaaaggacgaaacaccgggtgtcggaaaccgctaattcaggggccgctacaaccggcctgtggcatccgcccaaacctgatgg

SEQ ID NO: 494 - DNA, 90 bp, synthetic construct
  source 1..90: mol_type = other DNA
acgcacactggacgcgcaaaaaaagttgtagcggcccctgaattagcggtttccgacaccccatcaggtttgggcggatgccacaggccg

SEQ ID NO: 495 - DNA, 89 bp, synthetic construct
  source 1..89: mol_type = other DNA
cttgtggaaaggacgaaacaccgagtttagcagattgggatttgtactctgaccggaaccaaatgctggtaacactgtggtccacaagg

SEQ ID NO: 496 - DNA, 90 bp, synthetic construct
  source 1..90: mol_type = other DNA
acgcacactggacgcgcaaaaaaagttccggtcagagtacaaatcccaatctgctaaactccttgtggaccacagtgttaccagcatttg

SEQ ID NO: 497 - DNA, 89 bp, synthetic construct
  source 1..89: mol_type = other DNA
cttgtggaaaggacgaaacaccgagtttagcagattgggatttgtactctgaccggaacgtgcagctcctcagtcacaatcagggaagc

SEQ ID NO: 498 - DNA, 90 bp, synthetic construct
  source 1..90: mol_type = other DNA
acgcacactggacgcgcaaaaaaagttccggtcagagtacaaatcccaatctgctaaactgcttccctgattgtgactgaggagctgcac

SEQ ID NO: 499 - DNA, 89 bp, synthetic construct
  source 1..89: mol_type = other DNA
cttgtggaaaggacgaaacaccgagtttagcagattgggatttgtactctgaccggaacgcggtgggctcggtcctgcgcttgcaggtc

SEQ ID NO: 500 - DNA, 90 bp, synthetic construct
  source 1..90: mol_type = other DNA
acgcacactggacgcgcaaaaaaagttccggtcagagtacaaatcccaatctgctaaactgacctgcaagcgcaggaccgagcccaccgc

SEQ ID NO: 501 - DNA, 89 bp, synthetic construct
  source 1..89: mol_type = other DNA
cttgtggaaaggacgaaacaccgagtttagcagattgggatttgtactctgaccggaactccgagtccttcacccgtttgatctgctcc

SEQ ID NO: 502 - DNA, 90 bp, synthetic construct
  source 1..90: mol_type = other DNA
acgcacactggacgcgcaaaaaaagttccggtcagagtacaaatcccaatctgctaaactggagcagatcaaacgggtgaaggactcgga

SEQ ID NO: 503 - DNA, 89 bp, synthetic construct
  source 1..89: mol_type = other DNA
cttgtggaaaggacgaaacaccgagtttagcagattgggatttgtactctgaccggaacgtttctggcagttctcctctcctgcacccc

SEQ ID NO: 504 - DNA, 90 bp, synthetic construct
  source 1..90: mol_type = other DNA
acgcacactggacgcgcaaaaaaagttccggtcagagtacaaatcccaatctgctaaactggggtgcaggagaggagaactgccagaaac

SEQ ID NO: 505 - DNA, 89 bp, synthetic construct
  source 1..89: mol_type = other DNA
cttgtggaaaggacgaaacaccgagtttagcagattgggatttgtactctgaccggaaccggcctgtggcatccgcccaaacctgatgg

SEQ ID NO: 506 - DNA, 90 bp, synthetic construct
  source 1..90: mol_type = other DNA
acgcacactggacgcgcaaaaaaagttccggtcagagtacaaatcccaatctgctaaactccatcaggtttgggcggatgccacaggccg

SEQ ID NO: 507 - DNA, 89 bp, synthetic construct
  source 1..89: mol_type = other DNA
cttgtggaaaggacgaaacaccgagtttagcagattgggatttgtactctgaccggaactcttccggacatcctgaaggtgctgctcca

SEQ ID NO: 508 - DNA, 90 bp, synthetic construct
  source 1..90: mol_type = other DNA
acgcacactggacgcgcaaaaaaagttccggtcagagtacaaatcccaatctgctaaacttggagcagcaccttcaggatgtccggaaga

SEQ ID NO: 509 - DNA, 89 bp, synthetic construct
  source 1..89: mol_type = other DNA
cttgtggaaaggacgaaacaccgagtttagcagattgggatttgtactctgaccggaactccaatgcaggcaatctgttgccgcctctt

SEQ ID NO: 510 - DNA, 90 bp, synthetic construct
  source 1..90: mol_type = other DNA
acgcacactggacgcgcaaaaaaagttccggtcagagtacaaatcccaatctgctaaactaagaggcggcaacagattgcctgcattgga

SEQ ID NO: 511 - DNA, 89 bp, synthetic construct
  source 1..89: mol_type = other DNA
cttgtggaaaggacgaaacaccgagtttagcagattgggatttgtactctgaccggaaccttggtgatacacctcggtctcaaaggtga

SEQ ID NO: 512 - DNA, 90 bp, synthetic construct
  source 1..90: mol_type = other DNA
acgcacactggacgcgcaaaaaaagttccggtcagagtacaaatcccaatctgctaaacttcacctttgagaccgaggtgtatcaccaag

SEQ ID NO: 513 - DNA, 89 bp, synthetic construct
  source 1..89: mol_type = other DNA
cttgtggaaaggacgaaacaccgagtttagcagattgggatttgtactctgaccggaaccaagaatacattatgggtactgaagcaact

SEQ ID NO: 514 - DNA, 90 bp, synthetic construct
  source 1..90: mol_type = other DNA
acgcacactggacgcgcaaaaaaagttccggtcagagtacaaatcccaatctgctaaactagttgcttcagtacccataatgtattcttg

SEQ ID NO: 515 - DNA, 89 bp, synthetic construct
  source 1..89: mol_type = other DNA
cttgtggaaaggacgaaacaccgagtttagcagattgggatttgtactctgaccggaacgtttcagtccctgcttccctatcactgtct

SEQ ID NO: 516 - DNA, 90 bp, synthetic construct
  source 1..90: mol_type = other DNA
acgcacactggacgcgcaaaaaaagttccggtcagagtacaaatcccaatctgctaaactagacagtgatagggaagcagggactgaaac

SEQ ID NO: 517 - DNA, 89 bp, synthetic construct
  source 1..89: mol_type = other DNA
cttgtggaaaggacgaaacaccgagtttagcagattgggatttgtactctgaccggaactgccgtggatgatcacagggttgatagttg

SEQ ID NO: 518 - DNA, 90 bp, synthetic construct
  source 1..90: mol_type = other DNA
acgcacactggacgcgcaaaaaaagttccggtcagagtacaaatcccaatctgctaaactcaactatcaaccctgtgatcatccacggca

SEQ ID NO: 519 - DNA, 89 bp, synthetic construct
  source 1..89: mol_type = other DNA
cttgtggaaaggacgaaacaccgagtttagcagattgggatttgtactctgaccggaactccactgtgttgagggcaatgaggacataa

SEQ ID NO: 520 - DNA, 90 bp, synthetic construct
  source 1..90: mol_type = other DNA
acgcacactggacgcgcaaaaaaagttccggtcagagtacaaatcccaatctgctaaactttatgtcctcattgccctcaacacagtgga

SEQ ID NO: 521 - DNA, 89 bp, synthetic construct
  source 1..89: mol_type = other DNA
cttgtggaaaggacgaaacaccgagtttagcagattgggatttgtactctgaccggaactggttgtggcagcagtcactgggggacttg

SEQ ID NO: 522 - DNA, 90 bp, synthetic construct
  source 1..90: mol_type = other DNA
acgcacactggacgcgcaaaaaaagttccggtcagagtacaaatcccaatctgctaaactcaagtcccccagtgactgctgccacaacca

SEQ ID NO: 523 - DNA, 89 bp, synthetic construct
  source 1..89: mol_type = other DNA
cttgtggaaaggacgaaacaccgagtttagcagattgggatttgtactctgaccggaacctaaatgccaccggcaggatgtggagatcg

SEQ ID NO: 524 - DNA, 90 bp, synthetic construct
  source 1..90: mol_type = other DNA
acgcacactggacgcgcaaaaaaagttccggtcagagtacaaatcccaatctgctaaactcgatctccacatcctgccggtggcatttag

SEQ ID NO: 525 - DNA, 89 bp, synthetic construct
  source 1..89: mol_type = other DNA
cttgtggaaaggacgaaacaccgagtttagcagattgggatttgtactctgaccggaactggatctgttcttgtgaatggaatgtcttc

SEQ ID NO: 526 - DNA, 90 bp, synthetic construct
  source 1..90: mol_type = other DNA
acgcacactggacgcgcaaaaaaagttccggtcagagtacaaatcccaatctgctaaactgaagacattccattcacaagaacagatcca

SEQ ID NO: 527 - DNA, 89 bp, synthetic construct
  source 1..89: mol_type = other DNA
cttgtggaaaggacgaaacaccgagtttagcagattgggatttgtactctgaccggaacactgcaaggcatttctgaaaccgacagtac

SEQ ID NO: 528 - DNA, 90 bp, synthetic construct
  source 1..90: mol_type = other DNA
acgcacactggacgcgcaaaaaaagttccggtcagagtacaaatcccaatctgctaaactgtactgtcggtttcagaaatgccttgcagt

SEQ ID NO: 529 - DNA, 89 bp, synthetic construct
  source 1..89: mol_type = other DNA
cttgtggaaaggacgaaacaccgagtttagcagattgggatttgtactctgaccggaactccatatttgaggagagttacttggtcgtt

SEQ ID NO: 530 - DNA, 90 bp, synthetic construct
  source 1..90: mol_type = other DNA
acgcacactggacgcgcaaaaaaagttccggtcagagtacaaatcccaatctgctaaactaacgaccaagtaactctcctcaaatatgga

SEQ ID NO: 531 - DNA, 90 bp, synthetic construct
  source 1..90: mol_type = other DNA
cttgtggaaaggacgaaacaccggtagaaatgagtacaaagcgatagagagcttaataaccaaatgctggtaacactgtggtccacaagg

SEQ ID NO: 532 - DNA, 91 bp, synthetic construct
  source 1..91: mol_type = other DNA
acgcacactggacgcgcaaaaaaagttattaagctctctatcgctttgtactcatttctacccttgtggaccacagtgttaccagcatttg

SEQ ID NO: 533 - DNA, 90 bp, synthetic construct
  source 1..90: mol_type = other DNA
cttgtggaaaggacgaaacaccggtagaaatgagtacaaagcgatagagagcttaataacgtgcagctcctcagtcacaatcagggaagc

SEQ ID NO: 534 - DNA, 91 bp, synthetic construct
  source 1..91: mol_type = other DNA
acgcacactggacgcgcaaaaaaagttattaagctctctatcgctttgtactcatttctacgcttccctgattgtgactgaggagctgcac

SEQ ID NO: 535 - DNA, 85 bp, synthetic construct
  source 1..85: mol_type = other DNA
cttgtggaaaggacgaaacaccgaactcggaaggattcagaagaagctttcatctcaaatgctggtaacactgtggtccacaagg

SEQ ID NO: 536 - DNA, 86 bp, synthetic construct
  source 1..86: mol_type = other DNA
acgcacactggacgcgcaaaaaaaagatgaaagcttcttctgaatccttccgagttccttgtggaccacagtgttaccagcatttg

SEQ ID NO: 537 - DNA, 85 bp, synthetic construct
  source 1..85: mol_type = other DNA
cttgtggaaaggacgaaacaccgaactcggaaggattcagaagaagctttcatctgtgcagctcctcagtcacaatcagggaagc

SEQ ID NO: 538 - DNA, 86 bp, synthetic construct
  source 1..86: mol_type = other DNA
acgcacactggacgcgcaaaaaaaagatgaaagcttcttctgaatccttccgagttgcttccctgattgtgactgaggagctgcac

SEQ ID NO: 539 - DNA, 85 bp, synthetic construct
  source 1..85: mol_type = other DNA
cttgtggaaaggacgaaacaccgaactcggaaggattcagaagaagctttcatctgcggtgggctcggtcctgcgcttgcaggtc

SEQ ID NO: 540 - DNA, 86 bp, synthetic construct
  source 1..86: mol_type = other DNA
acgcacactggacgcgcaaaaaaaagatgaaagcttcttctgaatccttccgagttgacctgcaagcgcaggaccgagcccaccgc

SEQ ID NO: 541 - DNA, 85 bp, synthetic construct
  source 1..85: mol_type = other DNA
cttgtggaaaggacgaaacaccgaactcggaaggattcagaagaagctttcatcttccgagtccttcacccgtttgatctgctcc

SEQ ID NO: 542 - DNA, 86 bp, synthetic construct
  source 1..86: mol_type = other DNA
acgcacactggacgcgcaaaaaaaagatgaaagcttcttctgaatccttccgagttggagcagatcaaacgggtgaaggactcgga

SEQ ID NO: 543 - DNA, 85 bp, synthetic construct
  source 1..85: mol_type = other DNA
cttgtggaaaggacgaaacaccgaactcggaaggattcagaagaagctttcatctgtttctggcagttctcctctcctgcacccc

SEQ ID NO: 544 - DNA, 86 bp, synthetic construct
  source 1..86: mol_type = other DNA
acgcacactggacgcgcaaaaaaaagatgaaagcttcttctgaatccttccgagttggggtgcaggagaggagaactgccagaaac

SEQ ID NO: 545 - DNA, 85 bp, synthetic construct
  source 1..85: mol_type = other DNA
cttgtggaaaggacgaaacaccgaactcggaaggattcagaagaagctttcatctcggcctgtggcatccgcccaaacctgatgg

SEQ ID NO: 546 - DNA, 86 bp, synthetic construct
  source 1..86: mol_type = other DNA
acgcacactggacgcgcaaaaaaaagatgaaagcttcttctgaatccttccgagttccatcaggtttgggcggatgccacaggccg

SEQ ID NO: 547 - DNA, 85 bp, synthetic construct
  source 1..85: mol_type = other DNA
cttgtggaaaggacgaaacaccgaactcggaaggattcagaagaagctttcatctccaggagattatgaaacaccaaagtggcat

SEQ ID NO: 548 - DNA, 86 bp, synthetic construct
  source 1..86: mol_type = other DNA
acgcacactggacgcgcaaaaaaaagatgaaagcttcttctgaatccttccgagttatgccactttggtgtttcataatctcctgg

SEQ ID NO: 549 - DNA, 85 bp, synthetic construct
  source 1..85: mol_type = other DNA
cttgtggaaaggacgaaacaccgaactcggaaggattcagaagaagctttcatctggacatcctgaaggtgctgctccagcatct

SEQ ID NO: 550 - DNA, 86 bp, synthetic construct
  source 1..86: mol_type = other DNA
acgcacactggacgcgcaaaaaaaagatgaaagcttcttctgaatccttccgagttagatgctggagcagcaccttcaggatgtcc

SEQ ID NO: 551 - DNA, 85 bp, synthetic construct
  source 1..85: mol_type = other DNA
cttgtggaaaggacgaaacaccgaactcggaaggattcagaagaagctttcatctaatgcaggcaatctgttgccgcctcttcca

SEQ ID NO: 552 - DNA, 86 bp, synthetic construct
  source 1..86: mol_type = other DNA
acgcacactggacgcgcaaaaaaaagatgaaagcttcttctgaatccttccgagtttggaagaggcggcaacagattgcctgcatt

SEQ ID NO: 553 - DNA, 85 bp, synthetic construct
  source 1..85: mol_type = other DNA
cttgtggaaaggacgaaacaccgaactcggaaggattcagaagaagctttcatcttgctgtaggggagaccaagaatacattatg

SEQ ID NO: 554 - DNA, 86 bp, synthetic construct
  source 1..86: mol_type = other DNA
acgcacactggacgcgcaaaaaaaagatgaaagcttcttctgaatccttccgagttcataatgtattcttggtctcccctacagca

SEQ ID NO: 555 - DNA, 85 bp, synthetic construct
  source 1..85: mol_type = other DNA
cttgtggaaaggacgaaacaccgaactcggaaggattcagaagaagctttcatctttctgctgtgcccttatctggaaacattga

SEQ ID NO: 556 - DNA, 86 bp, synthetic construct
  source 1..86: mol_type = other DNA
acgcacactggacgcgcaaaaaaaagatgaaagcttcttctgaatccttccgagtttcaatgtttccagataagggcacagcagaa

SEQ ID NO: 557 - DNA, 85 bp, synthetic construct
  source 1..85: mol_type = other DNA
cttgtggaaaggacgaaacaccgaactcggaaggattcagaagaagctttcatcttactgttattgggaagccgtcctcttctgc

SEQ ID NO: 558 - DNA, 86 bp, synthetic construct
  source 1..86: mol_type = other DNA
acgcacactggacgcgcaaaaaaaagatgaaagcttcttctgaatccttccgagttgcagaagaggacggcttcccaataacagta

SEQ ID NO: 559 - DNA, 85 bp, synthetic construct
  source 1..85: mol_type = other DNA
cttgtggaaaggacgaaacaccgagatgaaagcttcttctgaatccttccgagttcaaatgctggtaacactgtggtccacaagg

SEQ ID NO: 560 - DNA, 86 bp, synthetic construct
  source 1..86: mol_type = other DNA
acgcacactggacgcgcaaaaaaaaactcggaaggattcagaagaagctttcatctccttgtggaccacagtgttaccagcatttg

SEQ ID NO: 561 - DNA, 85 bp, synthetic construct
  source 1..85: mol_type = other DNA
cttgtggaaaggacgaaacaccgagatgaaagcttcttctgaatccttccgagttgtgcagctcctcagtcacaatcagggaagc

SEQ ID NO: 562 - DNA, 86 bp, synthetic construct
  source 1..86: mol_type = other DNA
acgcacactggacgcgcaaaaaaaaactcggaaggattcagaagaagctttcatctgcttccctgattgtgactgaggagctgcac